(19) United States
(12) Patent Application Publication
(10) Pub. No.: US 2023/0093678 A1
(43) Pub. Date: Mar. 23, 2023

(54) PEPTIDE IMMUNOGENS TARGETING PCSK9 AND FORMULATIONS THEREOF FOR PREVENTION AND TREATMENT OF PCSK9-MEDIATED DISORDERS

(71) Applicants: UBI IP HOLDINGS, Grand Cayman (KY); UBI US HOLDINGS, LLC, Hauppauge, NY (US)

(72) Inventors: Chang Yi Wang, Cold Spring Harbor, NY (US); Feng Lin, Hauppauge, NY (US)

(21) Appl. No.: 17/795,734

(22) PCT Filed: Jan. 28, 2021
(86) PCT No.: PCT/US2021/015423

§ 371 Date: Jul. 27, 2022

Related U.S. Application Data
(60) Provisional application No. 62/966,645, filed on Jan. 28, 2020.

(51) Int. Cl. A61K 39/00; C07K 16/40; A61P 9/10

(52) U.S. Cl. CPC A61K 39/0005; C07K 16/40; A61P 9/10; A61K 2039/55561; A61K 2039/6031

(57) ABSTRACT

The present disclosure is directed to peptide immunogen constructs targeting the catalytic domain of the PCSK9 protein, compositions containing the constructs, antibodies elicited by the constructs, and methods for making and using the constructs and compositions thereof. The disclosed peptide immunogen constructs have more than about 20 amino acids and contain (a) a B cell epitope having about more than about 7 contiguous amino acid residues from the PCSK9 and LDL-R receptor binding regions of the catalytic domain of the PCSK9 protein; (b) a heterologous Th epitope; and (c) an optional heterologous spacer. The disclosed PCSK9 peptide immunogen constructs stimulate the generation of highly specific antibodies directed to PCSK9 sites that are binding to LDL-R to allow for the prevention and/or treatment of patients with PCSK9 mediated disorders including an increased serum level of low-density lipoprotein cholesterol (LDL-C) and CV events.

The present application is a PCT International Application that claims the benefit of U.S. Provisional Application Ser. No. 62/966,645, filed Jan. 28, 2020, which is incorporated herein by reference in its entirety.

### FIELD OF THE INVENTION

This disclosure relates to peptide immunogen constructs targeting Proprotein Convertase Subtilisin-Kexin type 9 (PCSK9) and formulations thereof for prevention and treatment of patients with PCSK9 mediated disorders including an increased serum level of low-density lipoprotein cholesterol (LDL-C) and cardiovascular events.

### BACKGROUND OF THE INVENTION

Cardiovascular (CV) disease is the leading cause of mortality worldwide, accounting for an estimated 31.5% of all global deaths in 2013. In particular, there is a high burden of atherosclerotic CV disease (ASCVD), which can manifest as coronary heart disease (CHD), cerebrovascular disease, and peripheral artery disease. An increased serum level of low-density lipoprotein cholesterol (LDL-C) is an independent risk factor for ASCVD and clinical trial data have demonstrated a relationship between lowering LDL-C and reductions in CV risk. Consequently, reducing LDL-C is a key strategy for primary and secondary prevention of ASCVD.

The cornerstone therapy for LDL-C lowering has been statins, which inhibit 3-hydroxy-3-methylglutaryl-coenzyme A (HMG-CoA) reductase. Large-scale randomized trials have proven the efficacy of statins in reducing the risk of major vascular events by about 25% for each mmol/L reduction in LDL-C per year of statin use, after the first year of use, with greater absolute benefit of statin therapy for higher risk patients. In contrast, nonstatin lipid-lowering therapies, such as niacin and cholesteryl ester transfer protein inhibitors, have shown no CV benefit or have even increased the risk of CV events and mortality. Combination treatment with statin and ezetimibe to lower LDL-C modestly improve CV outcomes in patients with acute coronary syndrome and also suggest additional clinical benefit for reduction of LDL-C to levels below the prior practice guideline target of 70 mg/dl. Despite the proven efficacy of statins to reduce LDL-C levels and CV events, additional therapies are needed. Even with statin treatment, some patients have high residual CV risk due to inadequate lowering of LDL-C levels or persistent non-LDL-C-related dyslipidemia. Furthermore, adverse effects, including myopathy (ranging from mild myalgias to severe rhabdomyolysis), new-onset or worsening diabetes, and possibly hemorrhagic stroke, may limit statin use or the ability to attain goal-effective statin doses in certain patients. Recently, another class of drugs inhibiting PCSK9 has been developed for the treatment of hyperlipidemia.

PCSK9 was first identified in 2001 when elevated protein levels were found in studies of cerebellar neuron apoptosis. The protein was initially named neural apoptosis-regulated convertase 1, and the gene was characterized in 2003. The gene for PCSK9 is located on human chromosome 1p32 and encodes a 692-amino-acid serine protease. PCSK9 (GenBank Accession No.: EAX06660) has the amino acid sequence of SEQ ID NO: 1 as shown in both Table 1 and FIG. 2. PCSK9 is primarily expressed in the liver, but lower levels of protein expression have also been found in the intestine, kidney, and central nervous system. Transcription of PCSK9 is primarily regulated by sterol regulatory element-binding protein-2 (SREBP-2). In the hepatocyte, PCSK9 is synthesized as a zymogen, requiring activation by another enzyme; it is composed of a signal peptide (residues 1-30), a prodomain (residues 31-152), a catalytic domain (residues 153-454), and a C-terminal (CT)-domain (455-692) (Holla, Ø. L., et al., 2011). In the endoplasmic reticulum, PCSK9 undergoes cleavage of its signal peptide and undergoes co-translational autocatalytic cleavage into a prodomain and a mature protein, the latter of which is required for secretion from the endoplasmic reticulum to the Golgi body. Unique to PCSK9, the prodomain remains associated with the mature protein, facilitating protein folding, preventing enzyme activity by blocking access to the catalytic site, and chaperoning PCSK9 through the secretory pathway.

PCSK9 circulates in plasma, binds to cell surface low-density lipoprotein receptors (LDL-R), is internalized, and then targets the receptors to lysosomal degradation. Binding of PCSK9 to the epidermal growth factor precursor homology domain A (EGF-A) repeat of the LDL-R is mediated by a patch of residues on the catalytic domain of PCSK9. FIG. 3 identifies amino acid residues on PCSK9 and LDL-R that interface. The catalytic domain of PCSK9 is responsible for both autocatalytic cleavage and binding of PCSK9 to the LDL-R.

Concurrent studies in patients with familial hypercholesterolemia provided insight into the clinical importance of PCSK9, with gain-of-function mutations causing hypercholesterolemia. Murine models overexpressing PCSK9 demonstrated increases in levels of total cholesterol and non-high-density lipoprotein cholesterol (non-HDL-C) and reduced levels of the hepatic LDL-R, confirming a causal role for gain-of-function PCSK9 mutations in humans exhibiting the hypercholesterolemic phenotype. It has been shown that patients with loss-of-function PCSK9 mutations and associated hypocholesterolemia had a lower risk of CV disease.

PCSK9 plays an important role in LDL-C metabolism, as described by Chaudhary, R., et al. (2017). LDL-C bound to the LDL-R is internalized into hepatocytes through clathrin-coated vesicles, after which the acidic environment of the endosome causes dissociation of LDL-C from its receptor. Recycling vesicles return the LDL-R to the cell surface, while endosomes containing the LDL-C particles fuse with lysosomes, resulting in degradation of LDL-C, hydrolysis of cholesterol esters, and distribution of free cholesterol to the rest of the cell. At the hepatocyte plasma membrane, the catalytic domain of secreted PCSK9 associates with the LDL-R and is internalized, entering the endosomal pathway. The low pH of the endosome enhances the affinity of PCSK9 for the LDL-R, preventing the receptor from being recycled to the cell surface. Instead, the complex is directed to the lysosome, where both components are degraded. In addition, PCSK9 appears to enhance intracellular LDL-R degradation prior to secretion, as PCSK9 can complex with the LDL-R within the Golgi and direct the receptor to the lysosome for degradation instead of transport to the plasma membrane.

Multiple strategies of PCSK9 inhibition are currently under investigation. The first approach prevents binding of PCSK9 to the LDL-R. Examples of this approach include monoclonal antibodies. Monoclonal antibody therapeutic agents include evolocumab (REPATHA®), alirocumab (PRALUENT®), and bococizumab. These monoclonal antibodies bind the catalytic domain and prodomain of PCSK9, blocking interaction with the LDL-R and neutralizing PCSK9 activity. Studies have shown maximal suppression of circulating unbound PCSK9 within 4-8 h after administration of the monoclonal antibody, with reductions in LDL-C of about 65% in healthy subjects and about 60-80% in patients with hypercholesterolemia. PCSK9 inhibition can significantly lower LDL-C concentrations in humans, even on a background of statin therapy. Patient populations studied in clinical trials range from those at low CV risk to the very-high-CV-risk population of individuals homozygous for familial hypercholesterolemia (HoFH). Although such monoclonal anti-PCSK9 antibodies may prove efficacious in immunotherapy of PCSK-9 mediated disorders, they are expensive and must be administered monthly to maintain sufficient suppression of LDL-C serum levels and the clinical benefits derived therefrom. Two review articles (Hess, C., et al., 2018 and Chaudhary, R., et al., 2017) cite additional supporting documents for statements made in the above background section and are hereby incorporated by reference in their entireties.

A cost-effective immunotherapeutic treatment targeting PCSK9 molecule through vaccination approach that is safe and well tolerated remains an exciting new intervention and development for PCSK9 mediated disorders. Several approaches along this line of investigation have been explored including those by Brunner, S., et al. (U.S. Pat. No. 9,669,079) and Champion, R., et al. (U.S. Pat. No. 9,987,341), the disclosures of which are hereby incorporated by reference in their entireties.

There are a number of disadvantages and deficiencies associated with the traditional epitope-based vaccines where the methods for the preparation of immunogens involve complicated chemical conjugation procedures in which expensive pharmaceutical grade KLH or toxoid proteins are used as the T helper cell carrier. Most of the antibodies elicited by such immunogen preparations are directed against the carrier protein(s) and not the target B cell epitope(s).

In view of the economic and practical disadvantages with monoclonal anti-PCSK9 therapy and the complicated chemical conjugation procedures for the peptide/hapten-carrier protein immunogen preparations, there is clearly an unmet need to develop an efficacious immunotherapeutic composition capable of eliciting highly specific immune responses against the functional site(s) on PCSK9, that can be easily administered to patients, cost-effectively manufactured under stringent good manufacturing practices (GMP) for worldwide application to treat patients suffering from PCSK9 mediated disorders including an increased serum level of LDL-C and CV events.

### REFERENCES

1. CHANG, J. C. C., et al., “Adjuvant activity of incomplete Freund's adjuvant.” Advanced Drug Delivery Reviews, 32(3):173-186 (1998)
2. CHAUDHARY, R., et al., “PCSK9 inhibitors: A new era of lipid lowering therapy.” World J. Cardiol., 26:76-91 (2017)
3. FIELDS, G. B., et al., Chapter 3 in Synthetic Peptides: A User's Guide, ed. Grant, W. H. Freeman & Co., New York, N.Y., p. 77 (1992)
4. HESS, C., et al., “PCSK9 Inhibitors: Mechanisms of Action, Metabolic Effects, and Clinical Outcomes.” Annul. Rev. Med., 69:17.1-17.13 (2018)
5. HOLLA, Ø. L., et al., “Role of the C-terminal domain of PCSK9 in degradation of the LDL receptors.” J. Lipid Res., 52(10):1787-94 (2011)
6. TRAGGIAI, E., et al., “An efficient method to make human monoclonal antibodies from memory B cells: potent neutralization of SARS coronavirus.” Nature Medicine, 10:871-875 (2004)
7. U.S. Pat. No. 9,669,079, by BRUNNER, S., et al., “PCSK9 peptide combination vaccine and method of use.” (2017-06-06)
8. U.S. Pat. No. 9,987,341, by CHAMPION, R., et al., “PCSK9 vaccine.” (2018-06-05)
9. WO 1990/014837, by VAN NEST, G., et al., “Adjuvant formulation comprising a submicron oil droplet emulsion.” (1990-12-13)

### SUMMARY OF THE INVENTION

The present disclosure is directed to Proprotein Convertase Subtilisin-Kexin type 9 (PCSK9) and formulations thereof for prevention and treatment of PCSK9 mediated disorders, including an increased serum level of low-density lipoprotein cholesterol (LDL-C) and cardiovascular (CV) events. In particular, the present disclosure is directed to peptide immunogen constructs containing B cell epitopes from the catalytic domain of PCSK9, compositions containing the peptide immunogen constructs, methods of making and using the peptide immunogen constructs, and antibodies produced by the peptide immunogen constructs.

One aspect of the present disclosure is directed to B cell epitopes from the catalytic domain of PCSK9 (residues 153-454 of SEQ ID NO: 1). The disclosed B cell epitope peptides contain between about 7 to about 30 amino acids from the catalytic domain of the PCSK9 protein. In certain embodiments, the B cell epitope peptides have the amino acid sequences of SEQ ID NOs: 2-9, as shown in Table 1.

The disclosed B cell epitope peptides derived from the catalytic domain of PCSK9 can be linked through an optional heterologous spacer to a heterologous T helper cell (Th) epitope peptide to form a peptide immunogen construct. In certain embodiments, the heterologous spacer is any molecule or chemical structure capable of linking two amino acids and/or peptides together, which can include a chemical compound, a naturally occurring amino acid, a non-naturally occurring amino acid, or any combination thereof. The heterologous Th epitope can be any Th epitope that is capable of enhancing the immune response to the B cell epitope. In certain embodiments, the Th epitope is derived from pathogen proteins having the amino acid sequences of SEQ ID NOs: 13-64, as shown in Table 2.

The disclosed peptide immunogen constructs contain the PCSK9 B cell epitope peptide covalently linked, at either the N- or C-terminus through an optional heterologous spacer to the heterologous Th epitope. The disclosed peptide immunogen constructs, containing the B cell epitope and Th epitope, have 20 or more total amino acids. In certain embodiments, the peptide immunogen constructs have the amino acid sequences of SEQ ID NOs: 65-107, as shown in Table 3.

The disclosed PCSK9 peptide immunogen constructs, containing both designed B cell- and Th-epitope peptides, act together to stimulate the generation of highly specific antibodies directed against PCSK9 functional sites, including the PCSK9 and LDL-R receptor binding region located in the catalytic domain of the PCSK9 molecule. The antibodies generated from the disclosed peptide immunogen constructs provide therapeutic immune responses to patients with PCSK9 mediated disorders, including an increased serum level of LDL-C and CV events.

Another aspect of the present disclosure is directed to peptide compositions, including pharmaceutical compositions, containing a PCSK9 peptide immunogen construct. The compositions can contain one or more PCSK9 peptide immunogen constructs, pharmaceutically acceptable delivery carriers, adjuvants, and/or be formulated into a stabilized immunostimulatory complex using a CpG oligomer. In certain embodiments, the mixture of PCSK9 peptide immunogen constructs have heterologous Th epitopes derived from different pathogens that can be used to allow coverage of as broad a genetic background in patients leading to a higher percentage in responder rate upon immunization for the prevention and/or treatment of patients with PCSK9 mediated disorders, including an increased serum level of low-density lipoprotein cholesterol (LDL-C) and CV events.

The present disclosure is also directed to antibodies against the disclosed PCSK9 peptide immunogen constructs. In particular, the PCSK9 peptide immunogen constructs of the present disclosure are able to stimulate the generation of highly specific functional antibodies that are cross-reactive with the full-length PCSK9 protein. The disclosed antibodies bind with high specificity to PCSK9 without much, if any, directed to the heterologous Th epitopes employed for immunogenicity enhancement, which is in sharp contrast to antibodies produced using conventional KLH or toxoid proteins or other biological carriers used for such peptide immunogenicity enhancement. Thus, the disclosed PCSK9 peptide immunogen constructs are capable of breaking the immune tolerance against self-PCSK9, with a high responder rate, compared to other peptide or protein immunogens. Based on their unique characteristics and properties, the disclosed antibodies elicited by the PCSK9 peptide immunogen constructs are capable of providing a prophylactic and immunotherapeutic approach to treating patients suffering from PCSK9 mediated disorders including an increased serum level of low-density lipoprotein cholesterol (LDL-C) and CV events.

In a further aspect, the present invention provides human monoclonal antibodies against the catalytic domain of the PCSK9 molecule involved in LDL-R binding region induced by patients receiving compositions containing PCSK9 peptide immunogen constructs of this disclosure. An efficient method to make human monoclonal antibodies from B cells isolated from the blood of a human patient is described by Traggiai, E., et al, 2004, which is incorporated by reference.

The present disclosure is also directed to methods of making and using the disclosed PCSK9 peptide immunogen constructs, compositions, and antibodies. The disclosed methods provide for the low-cost manufacture and quality control of PCSK9 peptide immunogen constructs and compositions containing the constructs. The disclosed methods are also directed to preventing and/or treating subjects predisposed to, or suffering from PCSK9 mediated disorders including an increased serum level of low-density lipoprotein cholesterol (LDL-C) and CV events using the disclosed PCSK9 peptide immunogen constructs and/or antibodies elicited from the PCSK9 peptide immunogen constructs. The disclosed methods also include dosing regimens, dosage forms, and routes for administering the PCSK9 peptide immunogen constructs to prevent and/or treat PCSK9 mediated disorders including an increased serum level of low-density lipoprotein cholesterol (LDL-C) and CV events in a subject.

### BRIEF DESCRIPTION OF THE DRAWINGS

FIG. 1 depicts pathways from discovery to commercialization of high precision PCSK9 designer peptide immunogen constructs and formulations thereof for the treatment of patients with PCSK9 mediated disorders including an increased serum level of low-density lipoprotein cholesterol (LDL-C) and CV events.

FIG. 2 identifies the sequence of PCSK9 consisting of 692 amino acid residues (SEQ ID NO: 1), of which residues 1-30 constitute the signal peptide. The remainder of the protein is commonly divided into three domains. Residues 31-152 constitute the prodomain, residues 153-454 constitute the catalytic domain, and residues 455-692 constitute the C Terminal (CT)-domain. Binding of PCSK9 to the EGF-A repeat of the LDL-R is mediated by a patch of residues on the catalytic domain of PCSK9. The catalytic domain of PCSK9 is responsible both for autocatalytic cleavage and for binding of PCSK9 to the LDL-R.

FIG. 3 identifies amino acid residues on PCSK9 and LDL-R that interface. These are regions within the PCSK9 catalytic domain where the PCSK9 peptide immunogen constructs of the current invention are designed around.

FIG. 4 depicts sequence alignment of human (SEQ ID NO: 111), monkey (SEQ ID NO: 112), mouse (SEQ ID NO: 113), rat (SEQ ID NO: 114), and guinea pig (SEQ ID NO: 115) PCSK9 catalytic domain.

FIG. 5 depicts PCSK9 peptide immunogen constructs (SEQ ID NOs: 65-76) designed with B cell epitopes derived from the PCSK9 catalytic domain around the sites where PCSK9 and LDL-R interface (SEQ ID NOs: 2-9).

FIG. 6 illustrates the immunoreactivity results with corresponding PCSK9 B cell epitope peptides from immunogenicity studies in guinea pigs immunized with representative PCSK9 peptide immunogen constructs (SEQ ID NOs: 65-76) in ISA51 formulation. Titers of guinea pig immune sera are expressed as Log₁₀ Titer. Immune sera are collected from 0, 3, 6, 9, and/or 12 wpi as shown in the respective panels.

FIG. 7 illustrates the cross-immunoreactivity results with full-length recombinant human PCSK9 protein from immunogenicity studies in guinea pigs immunized with representative PCSK9 peptide immunogen constructs (SEQ ID NOs: 65-76) in ISA51 formulation. Titers of guinea pig immune sera are expressed as Log₁₀ EC₅₀. Immune sera are collected from 0, 3, 6, 9, 12, and/or 15 wpi as shown in the respective panels along with accompanying tables on the left side.

FIGS. 8A-8B illustrate the average results of LDL-C level (mg/dL) from plasma collected at the respective weeks as shown from immunogenicity studies in guinea pigs (n=3 per group) immunized with representative PCSK9 peptide immunogen constructs in ISA51 formulations. FIG. 8A shows the results from guinea pigs immunized with peptide immunogen constructs from the N-terminal region (SEQ ID NOs: 65-67) and Central region (SEQ ID NOs: 75 and 76) of PCSK9. FIG. 8B shows the results from guinea pigs immunized with peptide immunogen constructs from the C-terminal region (SEQ ID NOs: 68-74) of PCSK9. Rate of LDL-C reduction expressed in % when compared to those of the Placebo group with blood samples collected at the corresponding time point (e.g., 0, 3, 6 wpi, etc.) for each group of the guinea pigs immunized with the corresponding PCSK9 peptide immunogen constructs (SEQ ID NOs: 65-76) are shown in the accompanying tables on the right side.

FIGS. 9A-9B illustrate the average results of total cholesterol (T-CHO) level (mg/dL) from plasma collected at the respective weeks as shown from immunogenicity studies in guinea pigs (n=3 per group) immunized with representative PCSK9 peptide immunogen constructs in ISA51 formulation. FIG. 9A shows the results from guinea pigs immunized with peptide immunogen constructs from the N-terminal region (SEQ ID NOs: 65-67) and Central region (SEQ ID NOs: 75 and 76) of PCSK9. FIG. 9B shows the results from guinea pigs immunized with peptide immunogen constructs from the C-terminal region (SEQ ID NOs: 68-74) of PCSK9. Rate of T-CHO reduction expressed in % when compared to those of the Placebo group with blood samples collected at the corresponding time point (e.g., 0, 3, 6 wpi, etc.) for each group of the guinea pigs immunized with the corresponding PCSK9 peptide immunogen constructs (SEQ ID Nos: 65-76) are shown in the accompanying tables on the right side.

FIGS. 10A-10C illustrate the results related to the inhibition of LDL-R degradation by polyclonal antibodies from guinea pigs immunized with PCSK9 peptide immunogen constructs. FIG. 10A shows the LDL assay procedure in the left panel of the figure and the LDL update ratio (indicative of LDL-R expression on the surface without being degraded due to PCSK9 binding) as bar graphs on the right panel in an antibody dose (0, 5, 15, 100, 500, 1,000, 1,250 μg/mL) dependent fashion from guinea pigs immunized with PCSK9 peptide immunogen constructs of SEQ ID NOs: 65 and 75. FIG. 10B shows the LDL update ratio as bar graphs in an antibody dose (0, 50, 250, and 500 μg/mL) dependent fashion from guinea pigs immunized with PCSK9 peptide immunogen constructs of SEQ ID NOs: 65, 75, 76, and 68-74. FIG. 10C shows the LDL update ratio as bar graphs in an antibody dose (1,250, 1,000, 500, 100, 15, 5, and 0 μg/mL) dependent fashion from guinea pigs immunized with PCSK9 peptide immunogen constructs of SEQ ID NOs: 65, 75, 76, and 70.

FIG. 11 shows the immunogenicity serum titer analysis of polyclonal antibodies elicited in guinea pigs immunized with PCSK9 peptide immunogens of SEQ ID NO: 66 or 67. The graphs shown in the right panel demonstrate that the antibodies elicited by the peptide immunogen constructs are highly reactive against the B cell epitopes of PCSK9 (SEQ ID NOs: 3 and 4) and not against the Th epitope of UBITh®1 or the CpG3 oligonucleotide.

FIG. 12 shows the reduction of sera T-CHO and LDL levels in guinea pigs immunized with PCSK9 peptide immunogens of SEQ ID NO: 65, 66, or 67.

### DETAILED DESCRIPTION OF THE INVENTION

The present disclosure is directed to Proprotein Convertase Subtilisin-Kexin type 9 (PCSK9) and formulations thereof for prevention and treatment of PCSK9 mediated disorders, including an increased serum level of low-density lipoprotein cholesterol (LDL-C) and CV events. In particular, the present disclosure is directed to peptide immunogen constructs containing B cell epitopes from the catalytic domain of PCSK9, compositions containing the peptide immunogen constructs, methods of making and using the peptide immunogen constructs, and antibodies produced by the peptide immunogen constructs.

One aspect of the present disclosure is directed to B cell epitopes from the catalytic domain of PCSK9 (residues 153-454 of SEQ ID NO: 1). The disclosed B cell epitope peptides contain between about 7 to about 30 amino acids from the catalytic domain of the PCSK9 protein. In certain embodiments, the B cell epitope peptides have the amino acid sequences of SEQ ID NOs: 2-9, as shown in Table 1.

The disclosed B cell epitope peptides derived from the catalytic domain of PCSK9 can be linked through an optional heterologous spacer to a heterologous T helper cell (Th) epitope peptide to form a peptide immunogen construct. In certain embodiments, the heterologous spacer is any molecule or chemical structure capable of linking two amino acids and/or peptides together, which can include a chemical compound, a naturally occurring amino acid, a non-naturally occurring amino acid, or any combination thereof. The heterologous Th epitope can be any Th epitope that is capable of enhancing the immune response to the B cell epitope. In certain embodiments, the Th epitope is derived from pathogen proteins having the amino acid sequences of SEQ ID NOs: 13-64, as shown in Table 2.

In certain embodiments, the heterologous Th epitopes employed to enhance the PCSK9 B cell epitope peptide are derived from natural pathogens EBV BPLF1 (SEQ ID NO: 51), EBV CP (SEQ ID NO: 48), Clostridium Tetani (SEQ ID NOs: 13, 16, 43, 45-47), Cholera Toxin (SEQ ID NO: 20), and Schistosoma mansoni (SEQ ID NO: 19), as well as those idealized artificial Th epitopes derived from Measles Virus Fusion protein (MVF 1 to 5) and Hepatitis B Surface Antigen (HBsAg 1 to 3) in the form of either single sequence or combinatorial sequences (e.g. SEQ ID NOs: 14, 21-38, and 53-64).

The disclosed peptide immunogen constructs contain the PCSK9 B cell epitope peptide covalently linked, at either the N- or C-terminus through an optional heterologous spacer to the heterologous Th epitope. The disclosed peptide immunogen constructs, containing the B cell epitope from PCSK9 and Th epitope, have 20 or more total amino acids. In certain embodiments, the peptide immunogen constructs have the amino acid sequences of SEQ ID NOs: 65-107, as shown in Table 3.

The disclosed PCSK9 peptide immunogen constructs, containing both designed B cell- and Th epitope peptides, act together to stimulate the generation of highly specific antibodies directed against PCSK9 functional sites, including the PCSK9 and LDL-R receptor binding region located in the catalytic domain of the PCSK9 molecule, offering therapeutic immune responses to patients with PCSK9 mediated disorders including an increased serum level of low-density lipoprotein cholesterol (LDL-C) and CV events.

Another aspect of the present disclosure is directed to peptide compositions containing a PCSK9 peptide immunogen construct. In some embodiments, the compositions contain one peptide immunogen construct. In other embodiments, peptide compositions comprise a mixture of PCSK9 peptide immunogen constructs. In certain embodiments, the mixture of PCSK9 peptide immunogen constructs have heterologous Th epitopes derived from different pathogens that can be used to allow coverage of as broad a genetic background in patients leading to a higher percentage in responder rate upon immunization for the prevention and/or treatment of patients with PCSK9 mediated disorders including an increased serum level of low-density lipoprotein cholesterol (LDL-C) and CV events.

Synergistic enhancement in PCSK9 immunogen constructs can be observed in the peptide compositions of this disclosure. The antibody response derived from the administration of such compositions containing PCSK9 peptide immunogen constructs was mostly (>90%) focused on the desired cross-reactivity against the PCSK9 site(s) or LDL-R receptor binding region peptides (SEQ ID NOs: 2-9) without much, if any, directed to the heterologous Th epitopes employed for immunogenicity enhancement. The immune response using peptide immunogen constructs containing the disclosed Th epitopes is in sharp contrast to standard methods that use a conventional carrier protein, such as KLH, toxoid, or other biological carriers for such peptide antigenicity enhancement.

The present disclosure is also directed to pharmaceutical compositions and formulations for the prevention and/or treatment of patients with PCSK9 mediated disorders, including an increased serum level of low-density lipoprotein cholesterol (LDL-C) and CV events. In some embodiments, pharmaceutical compositions are formulated into a stabilized immunostimulatory complex that is formed by mixing a CpG oligomer with a peptide composition containing a PCSK9 peptide immunogen construct, or a mixture of constructs, through electrostatic association. Such stabilized immunostimulatory complexes further enhance the PCSK9 peptide immunogenicity towards the desired cross-reactivity with the full-length PCSK9 protein.

In other embodiments, pharmaceutical compositions comprising the disclosed PCSK9 peptide immunogen construct, or mixture of constructs, are formulated with pharmaceutically acceptable delivery vehicles or adjuvants, such as mineral salts, including Alum gel (ALHYDROGEL) or Aluminum phosphate (ADJU-PHOS) to form a suspension formulation, or with MONTANIDE™ ISA 51 or 720 as adjuvant to form water-in-oil emulsions, that can be used for the prevention and/or treatment of patients with PCSK9 mediated disorders including an increased serum level of low-density lipoprotein cholesterol (LDL-C) and CV events.

The present disclosure is also directed to antibodies against the disclosed PCSK9 peptide immunogen constructs. In particular, the PCSK9 peptide immunogen constructs of the present disclosure are able to stimulate the generation of highly specific functional antibodies that are cross-reactive with the full-length PCSK9 protein. The disclosed antibodies bind with high specificity to PCSK9 without much, if any, directed to the heterologous Th epitopes employed for immunogenicity enhancement, which is in sharp contrast to antibodies produced using conventional KLH or toxoid proteins or other biological carriers used for such peptide immunogenicity enhancement. Thus, the disclosed PCSK9 peptide immunogen constructs are capable of breaking the immune tolerance against self-PCSK9, with a high responder rate, compared to other peptide or protein immunogens.

In some embodiments, the disclosed antibodies are directed against, and specifically bind to, the PCSK9 and LDL-R receptor binding sites on the catalytic domain of the PCSK9 molecule (e.g., SEQ ID NOs: 2-9) when the peptide immunogen constructs are administered to a subject. The highly specific antibodies elicited by these PCSK9 peptide immunogen constructs can inhibit PCSK9 and LDL-R receptor binding and the downstream cellular events including internalization and cellular processing of PCSK9 and LDL-R complex resulting in LDL-R degradation, leading to effective prevention and/or treatment of patients with PCSK9 mediated disorders including an increased serum level of low-density lipoprotein cholesterol (LDL-C) and CV events.

Based on their unique characteristics and properties, the disclosed antibodies elicited by the PCSK9 peptide immunogen constructs are capable of providing a prophylactic and immunotherapeutic approach to treating patients suffering from PCSK9 mediated disorders including an increased serum level of low-density lipoprotein cholesterol (LDL-C) and CV events.

In a further aspect, the present invention provides human monoclonal antibodies against the catalytic domain of the PCSK9 molecule involved in LDL-R binding region induced by patients receiving compositions containing PCSK9 peptide immunogen constructs of this disclosure. An efficient method to make human monoclonal antibodies from B cells isolated from the blood of a human patient is described by Traggiai, E., et al, 2004, which is incorporated by reference.

The present disclosure is also directed to methods of making the disclosed PCSK9 peptide immunogen constructs, compositions, and antibodies. The disclosed methods provide for the low cost manufacture and quality control of PCSK9 peptide immunogen constructs and compositions containing the constructs, which can be used in methods for treating patients suffering from PCSK9 mediated disorders including an increased serum level of low-density lipoprotein cholesterol (LDL-C) and CV events.

The present disclosure also includes methods for preventing and/or treating subjects predisposed to, or suffering from, PCSK9 mediated disorders including an increased serum level of low-density lipoprotein cholesterol (LDL-C) and CV events using the disclosed PCSK9 peptide immunogen constructs and/or antibodies elicited from the PCSK9 peptide immunogen constructs. The methods for preventing and/or treating patients with PCSK9 mediated disorders include administering to the subject a composition containing a disclosed PCSK9 peptide immunogen construct or mixture of constructs. In certain embodiments, the compositions utilized in the methods contain a disclosed PCSK9 peptide immunogen construct in the form of a stable immunostimulatory complex with negatively charged oligonucleotides, such as CpG oligomers, through electrostatic association, which can be further supplemented with an adjuvant.

The disclosed methods also include dosing regimens, dosage forms, and routes for administering the PCSK9 peptide immunogen constructs to prevent and/or treat patients with PCSK9 mediated disorders, including an increased serum level of low-density lipoprotein cholesterol (LDL-C) and CV events in a subject.

### General

The section headings used herein are for organizational purposes only and are not to be construed as limiting the subject matter described. All references or portions of references cited in this application are expressly incorporated by reference herein in their entirety for any purpose.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. Hence, the phrase “comprising A or B” means including A, or B, or A and B. It is further to be understood that all amino acid sizes, and all molecular weight or molecular mass values, given for polypeptides are approximate, and are provided for description. Although methods and materials similar or equivalent to those described herein can be used in the practice or testing of the disclosed method, suitable methods and materials are described below. All publications, patent applications, patents, and other references mentioned herein are incorporated by reference in their entirety. In case of conflict, the present specification, including explanations of terms, will control. In addition, the materials, methods, and examples disclosed herein are illustrative only and not intended to be limiting.

### PCSK9 Peptide Immunogen Construct

The present disclosure provides peptide immunogen constructs containing a B cell epitope peptide having about 7 to about 30 amino acids with an amino acid sequence from PCSK9 (SEQ ID NO: 1). In certain embodiments, the B cell epitope peptide has an amino acid sequence selected from SEQ ID NOs: 2-9, shown in Table 1, which are located at the N-terminal, Central, and C-terminal regions of the catalytic domain of PCSK9. In some embodiments, the B cell epitope peptide has an amino acid sequence from the PCSK9 and LDL-R receptor binding regions (e.g., SEQ ID NOs: 2-6 and 8-9, shown in Table 1).

The B cell epitope can be covalently linked to a heterologous T helper cell (Th) epitope derived from a pathogen protein (e.g., SEQ ID NOs: 13-64, as shown in Table 2) directly or through an optional heterologous spacer. These constructs, containing both designed B cell- and Th-epitopes act together to stimulate the generation of highly specific antibodies that are cross-reactive with full-length human PCSK9 (SEQ ID NO: 1).

The phrase “PCSK9 peptide immunogen construct” or “peptide immunogen construct”, as used herein, refers to a peptide with 20 or more amino acids containing (a) a B cell epitope having more than about 7 contiguous amino acid residues from the full-length PCSK9 protein (SEQ ID NO: 1); (b) a heterologous Th epitope; and (c) an optional heterologous spacer.

In certain embodiments, the PCSK9 peptide immunogen construct can be represented by the formulae:

(Th)ₘ-(A)ₙ-(PCSK9 functional B cell epitope peptide)-X

or

(PCSK9 functional B cell epitope peptide)-(A)ₙ-(Th)ₘ-X

or

(Th)ₘ-(A)ₙ-(PCSK9 functional B cell epitope peptide)-(A)ₙ-(Th)ₘ-X

wherein

Th is a heterologous T helper epitope;

A is a heterologous spacer;

(PCSK9 functional B cell epitope peptide) is a B cell epitope peptide having from 7 to 30 amino acid residues from PCSK9 that are involved in either receptor binding or receptor activation;

X is an α-COOH or α-CONH₂ of an amino acid;

m is from 1 to about 4; and

n is from 0 to about 10.

The PCSK9 peptide immunogen constructs of the present disclosure were designed and selected based on a number of rationales, including:
i. the PCSK9 B cell epitope peptide is non-immunogenic on its own to avoid autologous T cell activation;
ii. the PCSK9 B cell epitope peptide can be rendered immunogenic by using a protein carrier or a potent T helper epitope(s);
iii. when the PCSK9 B cell epitope peptide is rendered immunogenic and administered to a host, the host would:
   a. elicit high titer antibodies preferentially directed against the PCSK9 B cell epitope(s) and not the protein carrier or T helper epitope(s);
   b. break immune tolerance and generate highly specific antibodies having cross-reactivity with the PCSK9 protein (SEQ ID NO: 1);
   c. generate highly specific antibodies capable of inhibiting PCSK9 and LDL-R receptor binding along with the associated downstream cellular events resulting in degradation of LDL-R, thus causing an increase in LDL-C uptake by LDL-R expressing cells; and
   d. have lower levels of LDL-C and T-CHO in his/her plasma.

The disclosed PCSK9 peptide immunogen constructs and formulations thereof can effectively function as a pharmaceutical composition to prevent and/or treat subjects predisposed to, or suffering from, PCSK9 mediated disorders including an increased serum level of low-density lipoprotein cholesterol (LDL-C) and CV events in a subject.

The various components of the disclosed PCSK9 peptide immunogen construct are described in further detail below.

a. B Cell Epitope Peptide from PCSK9

The present disclosure is directed to Proprotein Convertase Subtilisin-Kexin type 9 (PCSK9) and formulations thereof for prevention and treatment of PCSK9 mediated disorders including an increased serum level of low-density lipoprotein cholesterol (LDL-C) and CV events. The present disclosure is also directed to peptide immunogen constructs containing B cell epitopes from the catalytic domain of PCSK9, compositions containing the peptide immunogen constructs, methods of making and using the peptide immunogen constructs, and antibodies produced by the peptide immunogen constructs.

The present disclosure is directed to a novel peptide composition for the generation of high titer antibodies with specificity for the Proprotein Convertase Subtilisin-Kexin type 9 (PCSK9) (e.g., SEQ ID NO: 1). The catalytic domain of PCSK9 (SEQ ID NO: 111) is used as the target for B cell epitopes. The site-specificity of the peptide immunogen constructs minimizes the generation of antibodies that are directed to irrelevant sites on other regions of PCSK9 or irrelevant sites on carrier proteins, thus providing a high safety factor.

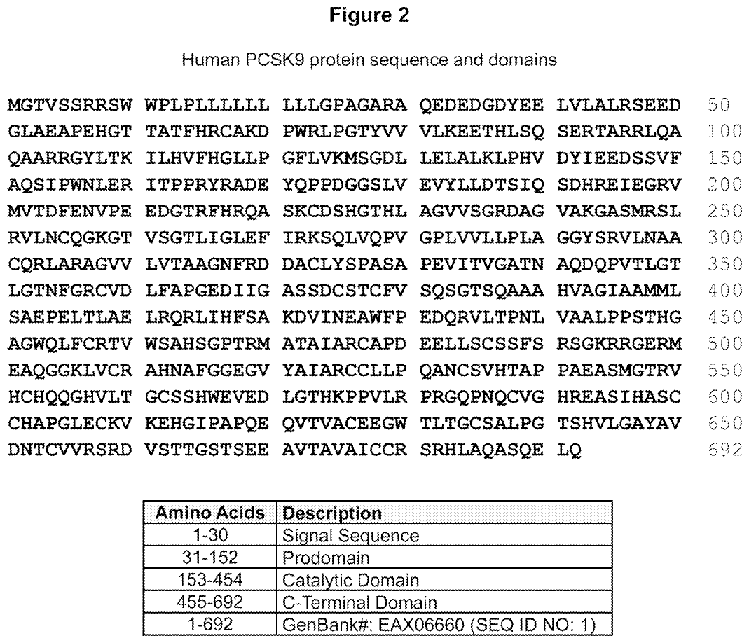

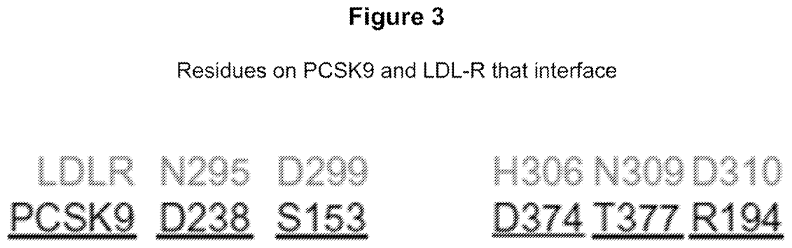

The gene for PCSK9 is located on human chromosome 1p32 and codes for a 692-amino-acid serine protease. PCSK9 (GenBank Accession No.: EAX06660) has the amino acid sequence of SEQ ID NO: 1 as shown in both Table 1 and FIG. 2. The PCSK9 protein is composed of a signal peptide (residues 1-30), a prodomain (residues 31-152), a catalytic domain (residues 153-454; SEQ ID NO: 111), and a C-terminal (CT)-domain (455-692), which consists of three modules, CM1 (residues 457-527), CM2 (residues 534-601), and CM3 (residues 608-692). Binding of PCSK9 to the EGF-A repeat of the LDL-R is mediated by a patch of residues on the catalytic domain of PCSK9. FIG. 3 identifies amino acid residues on PCSK9 and LDL-R that interface. The catalytic domain of PCSK9 is responsible both for autocatalytic cleavage and for binding of PCSK9 to the LDL-R.

One aspect of the present disclosure is to prevent and/or treat PCSK9-mediated disorders including an increased serum level of low-density lipoprotein cholesterol (LDL-C) and CV events in a subject. Thus, the present disclosure is directed to peptide immunogen constructs targeting the catalytic domain (SEQ ID NO: 111) of the full-length PCSK9 protein (SEQ ID NO: 1), as well as formulations thereof for the prevention and/or treatment of PCSK9 mediated disorders.

The B cell epitope portion of the PCSK9 peptide immunogen construct can contain between about 7 to about 30 amino acids from the catalytic domain (SEQ ID NO: 111) of the PCSK9 protein represented by SEQ ID NO: 1. In certain embodiments, the B cell epitope peptide, screened and selected based on design rationales, contains an amino acid sequence of SEQ ID NOs: 2-9, as shown in Table 1.

The PCSK9 B cell epitope peptide of the present disclosure also includes amino acid sequences that are immunologically functional analogues or homologues of PCSK9. Functional immunological analogues or homologues of the PCSK9 B cell epitope peptides include variants that retain substantially the same immunogenicity as the original peptide. Immunologically functional analogues can have a conservative substitution in an amino acid position; a change in overall charge; a covalent attachment to another moiety; or amino acid additions, insertions, or deletions; and/or any combination thereof. Examples of immunologically functional analogues are shown in Table 1 (e.g. SEQ ID NO: 2 vs 8; SEQ ID NO: 5 vs 9; and SEQ ID NO: 2, 3, and 4). Antibodies generated from peptide immunogen constructs containing the disclosed B cell epitopes from PCSK9 are highly specific and cross-reactive with the full-length human PCSK9 (SEQ ID NO: 1).

b. Heterologous T Helper Cell Epitopes (Th Epitopes)

The present disclosure provides peptide immunogen constructs containing a B cell epitope from PCSK9 covalently linked to a heterologous T helper cell (Th) epitope directly or through an optional heterologous spacer.

The heterologous Th epitope in the peptide immunogen construct enhances the immunogenicity of the PCSK9 B cell epitope portion, which facilitates the production of specific high titer antibodies directed against the optimized target PCSK9 B cell epitope peptide screened and selected based on design rationales.

The term “heterologous”, as used herein, refers to an amino acid sequence that is derived from an amino acid sequence that is not part of, or homologous with, the wild-type sequence of PCSK9. Thus, a heterologous Th epitope is a Th epitope derived from an amino acid sequence that is not naturally found in PCSK9 (i.e., the Th epitope is not autologous to PCSK9). Since the Th epitope is heterologous to PCSK9, the natural amino acid sequence of PCSK9 is not extended in either the N-terminal or C-terminal directions when the heterologous Th epitope is covalently linked to the PCSK9 B cell epitope peptide.

The heterologous Th epitope of the present disclosure can be any Th epitope that does not have an amino acid sequence naturally found in PCSK9. The Th epitope can also have promiscuous binding motifs to MHC class II molecules of multiple species. In certain embodiments, the Th epitope comprises multiple promiscuous MHC class II binding motifs to allow maximal activation of T helper cells leading to initiation and regulation of immune responses. The Th epitope is preferably immunosilent on its own, i.e. little, if any, of the antibodies generated by the PCSK9 peptide immunogen constructs will be directed towards the Th epitope, thus allowing a very focused immune response directed to the targeted B cell epitope peptide of the PCSK9 molecule.

Th epitopes of the present disclosure include, but are not limited to, amino acid sequences derived from foreign pathogens, as exemplified in Table 2 (e.g., SEQ ID NOs: 13-64). Further, the heterologous Th epitopes include idealized artificial Th epitopes (e.g., SEQ ID NOs: 14, 21, 25-29, 31-32, 34-35, 37-38, 53-56, 58-59, and 61-64) and combinatorial idealized artificial Th epitopes (e.g., SEQ ID NOs: 24, 30, 33, 36, 57, and 60). The combinatorial idealized artificial Th epitopes contain a mixture of amino acid residues represented at specific positions within the peptide framework based on the variable residues of homologues for that particular peptide. An assembly of combinatorial peptides can be synthesized in one process by adding a mixture of the designated protected amino acids, instead of one particular amino acid, at a specified position during the synthesis process. Such combinatorial heterologous Th epitope peptides assemblies can allow broad Th epitope coverage for animals having a diverse genetic background. Representative combinatorial sequences of heterologous Th epitope peptides include SEQ ID NOs: 24, 30, 33, 36, 57, and 60 which are shown in Table 2. Th epitope peptides of the present invention provide broad reactivity and immunogenicity to animals and patients from genetically diverse populations.

c. Heterologous Spacer

The disclosed PCSK9 peptide immunogen constructs optionally contain a heterologous spacer that covalently links the PCSK9 B cell epitope peptide to the heterologous T helper cell (Th) epitope.

As discussed above, the term “heterologous”, refers to an amino acid sequence that is derived from an amino acid sequence that is not part of, or homologous with, the natural type sequence of PCSK9. Thus, the natural amino acid sequence of PCSK9 is not extended in either the N-terminal or C-terminal directions when the heterologous spacer is covalently linked to the PCSK9 B cell epitope peptide because the spacer is heterologous to the PCSK9 sequence.

The spacer is any molecule or chemical structure capable of linking two amino acids and/or peptides together. The spacer can vary in length or polarity depending on the application. The spacer attachment can be through an amide- or carboxyl-linkage but other functionalities are possible as well. The spacer can include a chemical compound, a naturally occurring amino acid, or a non-naturally occurring amino acid.

The spacer can provide structural features to the PCSK9 peptide immunogen construct. Structurally, the spacer provides a physical separation of the Th epitope from the B cell epitope of the PCSK9 fragment. The physical separation by the spacer can disrupt any artificial secondary structures created by joining the Th epitope to the B cell epitope. Additionally, the physical separation of the epitopes by the spacer can eliminate interference between the Th cell and/or B cell responses. Furthermore, the spacer can be designed to create or modify a secondary structure of the peptide immunogen construct. For example, a spacer can be designed to act as a flexible hinge to enhance the separation of the Th epitope and B cell epitope. A flexible hinge spacer can also permit more efficient interactions between the presented peptide immunogen and the appropriate Th cells and B cells to enhance the immune responses to the Th epitope and B cell epitope. Examples of sequences encoding flexible hinges are found in the immunoglobulin heavy chain hinge region, which are often proline rich. One particularly useful flexible hinge that can be used as a spacer is provided by the sequence Pro-Pro-Xaa-Pro-Xaa-Pro (SEQ ID NO: 10), where Xaa is any amino acid, and preferably aspartic acid.

The spacer can also provide functional features to the PCSK9 peptide immunogen construct. For example, the spacer can be designed to change the overall charge of the PCSK9 peptide immunogen construct, which can affect the solubility of the peptide immunogen construct. Additionally, changing the overall charge of the PCSK9 peptide immunogen construct can affect the ability of the peptide immunogen construct to associate with other compounds and reagents. As discussed in further detail below, the PCSK9 peptide immunogen construct can be formed into a stable immunostimulatory complex with a highly charged oligonucleotide, such as CpG oligomers, through electrostatic association. The overall charge of the PCSK9 peptide immunogen construct is important for the formation of these stable immunostimulatory complexes.

Chemical compounds that can be used as a spacer include, but are not limited to, (2-aminoethoxy) acetic acid (AEA), 5-aminovaleric acid (AVA), 6-aminocaproic acid (Ahx), 8-amino-3,6-dioxaoctanoic acid (AEEA, mini-PEG1), 12-amino-4,7,10-trioxadodecanoic acid (mini-PEG2), 15-amino-4,7,10,13-tetraoxapenta-decanoic acid (mini-PEG3), trioxatridecan-succinamic acid (Ttds), 12-amino-dodecanoic acid, Fmoc-5-amino-3-oxapentanoic acid (O1Pen), and the like.

Naturally occurring amino acids include alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine and valine.

Non-naturally occurring amino acids include, but are not limited to, ε-N Lysine, β-alanine, ornithine, norleucine, norvaline, hydroxyproline, thyroxine, γ-amino butyric acid, homoserine, citrulline, aminobenzoic acid, 6-aminocaproic acid (Aca; 6-Aminohexanoic acid), hydroxyproline, mercaptopropionic acid (MPA), 3-nitro-tyrosine, pyroglutamic acid, and the like.

The spacer in the PCSK9 peptide immunogen construct can be covalently linked at either N- or C-terminal end of the Th epitope and the PCSK9 B cell epitope peptide. In some embodiments, the spacer is covalently linked to the C-terminal end of the Th epitope and to the N-terminal end of the PCSK9 B cell epitope peptide. In other embodiments, the spacer is covalently linked to the C-terminal end of the PCSK9 B cell epitope peptide and to the N-terminal end of the Th epitope. In certain embodiments, more than one spacer can be used, for example, when more than one Th epitope is present in the PCSK9 peptide immunogen construct. When more than one spacer is used, each spacer can be the same as each other or different. Additionally, when more than one Th epitope is present in the PCSK9 peptide immunogen construct, the Th epitopes can be separated with a spacer, which can be the same as, or different from, the spacer used to separate the Th epitope from the PCSK9 B cell epitope peptide. There is no limitation in the arrangement of the spacer in relation to the Th epitope or the PCSK9 B cell epitope peptide. In certain embodiments, the heterologous spacer is a naturally occurring amino acid or a non-naturally occurring amino acid. In other embodiments, the spacer contains more than one naturally occurring or non-naturally occurring amino acid. In specific embodiments, the spacer is Lys-, Gly-, Lys-Lys-Lys-, (α, ε-N)Lys, ε-N-Lys-Lys-Lys-Lys (SEQ ID NO: 11), or Lys-Lys-Lys-ε-N-Lys (SEQ ID NO: 12).

d. Specific Embodiments of the PCSK9 Peptide Immunogen Constructs

In certain embodiments, the PCSK9 peptide immunogen constructs can be represented by the following formulae:

(Th)ₘ-(A)ₙ-(PCSK9 functional B cell epitope peptide)-X

or

(PCSK9 functional B cell epitope peptide)-(A)ₙ-(Th)ₘ-X

or

(Th)ₘ-(A)ₙ-(PCSK9 functional B cell epitope peptide)-(A)ₙ-(Th)ₘ-X

wherein

Th is a heterologous T helper epitope;

A is a heterologous spacer;

(PCSK9 functional B cell epitope peptide) is a B cell epitope peptide having from 7 to 30 amino acid residues from the catalytic domain of PCSK9 that are involved in PCSK9 and LDL-R receptor binding;

X is an α-COOH or α-CONH₂ of an amino acid;

m is from 1 to about 4; and

n is from 0 to about 10.

The B cell epitope peptide can contain between about 7 to about 30 amino acids from the catalytic domain (SEQ ID NO: 111) of the full-length PCSK9 protein represented by SEQ ID NO: 1. In certain embodiments, the B cell epitope peptide has an amino acid sequence selected from SEQ ID NOs: 2-9, shown in Table 1, which are located at the N-terminal, Central, and C-terminal regions of the catalytic domain of PCSK9. In some embodiments, the B cell epitope peptide has an amino acid sequence from the PCSK9 and LDL-R receptor binding regions (e.g., SEQ ID NOs: 2-6 and 8-9, shown in Table 1).

The heterologous Th epitope in the PCSK9 peptide immunogen construct has an amino acid sequence selected from any of SEQ ID NOs: 13-64, and combinations thereof, shown in Table 2. In some embodiments, more than one Th epitope is present in the PCSK9 peptide immunogen construct.

The optional heterologous spacer is selected from any of Lys-, Gly-, Lys-Lys-Lys-, (α, ε-N)Lys, Pro-Pro-Xaa-Pro-Xaa-Pro (SEQ ID NO: 10), ε-N-Lys-Lys-Lys-Lys (SEQ ID NO: 11), Lys-Lys-Lys-ε-N-Lys (SEQ ID NO: 12), and any combination thereof, where Xaa is any amino acid, but preferably aspartic acid. In specific embodiments, the heterologous spacer is ε-N-Lys-Lys-Lys-Lys (SEQ ID NO: 11) or Lys-Lys-Lys-ε-N-Lys (SEQ ID NO: 12).

In certain embodiments, the PCSK9 peptide immunogen construct has an amino acid sequence selected from any of SEQ ID NOs: 65-107, as shown in Table 3.

The PCSK9 peptide immunogen constructs comprising Th epitopes are produced simultaneously in a single solid-phase peptide synthesis in tandem with the PCSK9 fragment. Th epitopes also include immunological analogues of Th epitopes. Immunological Th analogues include immune-enhancing analogues, cross-reactive analogues and segments of any of these Th epitopes that are sufficient to enhance or stimulate an immune response to the PCSK9 B cell epitope peptide.

The Th epitope in the PCSK9 peptide immunogen construct can be covalently linked at either N- or C-terminal end of the PCSK9 B cell epitope peptide. In some embodiments, the Th epitope is covalently linked to the N-terminal end of the PCSK9 B cell epitope peptide. In other embodiments, the Th epitope is covalently linked to the C-terminal end of the PCSK9 B cell epitope peptide. In certain embodiments, more than one Th epitope is covalently linked to the PCSK9 B cell epitope peptide. When more than one Th epitope is linked to the PCSK9 B cell epitope peptide, each Th epitope can have the same amino acid sequence or different amino acid sequences. In addition, when more than one Th epitope is linked to the PCSK9 B cell epitope peptide, the Th epitopes can be arranged in any order. For example, the Th epitopes can be consecutively linked to the N-terminal end of the PCSK9 B cell epitope peptide, or consecutively linked to the C-terminal end of the PCSK9 B cell epitope peptide, or a Th epitope can be covalently linked to the N-terminal end of the PCSK9 B cell epitope peptide while a separate Th epitope is covalently linked to the C-terminal end of the PCSK9B cell epitope peptide. There is no limitation in the arrangement of the Th epitopes in relation to the PCSK9 B cell epitope peptide.

In some embodiments, the Th epitope is covalently linked to the PCSK9 B cell epitope peptide directly. In other embodiments, the Th epitope is covalently linked to the PCSK9 fragment through a heterologous spacer.

e. Variants, Homologues, and Functional Analogues

Variants and analogues of the above immunogenic peptide constructs that induce and/or cross-react with antibodies to the preferred PCSK9 B cell epitope peptides can also be used. Analogues, including allelic, species, and induced variants, typically differ from naturally occurring peptides at one, two, or a few positions, often by virtue of conservative substitutions. Analogues typically exhibit at least 75%, 80%, 85%, 90%, or 95% sequence identity with natural peptides. Some analogues also include unnatural amino acids or modifications of N- or C-terminal amino acids at one, two, or a few positions.

Variants that are functional analogues can have a conservative substitution in an amino acid position; a change in overall charge; a covalent attachment to another moiety; or amino acid additions, insertions, or deletions; and/or any combination thereof.

Conservative substitutions are when one amino acid residue is substituted for another amino acid residue with similar chemical properties. For example, the nonpolar (hydrophobic) amino acids include alanine, leucine, isoleucine, valine, proline, phenylalanine, tryptophan and methionine; the polar neutral amino acids include glycine, serine, threonine, cysteine, tyrosine, asparagine, and glutamine; the positively charged (basic) amino acids include arginine, lysine and histidine; and the negatively charged (acidic) amino acids include aspartic acid and glutamic acid.

In a particular embodiment, the functional analogue has at least 50% identity to the original amino acid sequence. In another embodiment, the functional analogue has at least 80% identity to the original amino acid sequence. In yet another embodiment, the functional analogue has at least 85% identity to the original amino acid sequence. In still another embodiment, the functional analogue has at least 90% identity to the original amino acid sequence.

Functional immunological analogues of the Th epitope peptides are also effective and included as part of the present invention. Functional immunological Th analogues can include conservative substitutions, additions, deletions and insertions of from one to about five amino acid residues in the Th epitope which do not essentially modify the Th-stimulating function of the Th epitope. The conservative substitutions, additions, and insertions can be accomplished with natural or non-natural amino acids, as described above for the PCSK9 B cell epitope peptide. Table 2 identifies another variation of a functional analogue for Th epitope peptide. In particular, SEQ ID NOs: 14 and 21 of MvF1 and MvF2 Th are functional analogues of SEQ ID NOs: 31-33 and 37 of MvF4 and MvF5, respectively, in that they differ in the amino acid frame by the deletion (SEQ ID NOs: 14 and 21) or the inclusion (SEQ ID NOs: 31-33 and 37) of two amino acids each at the N- and C-termini. The differences between these two series of analogous sequences would not affect the function of the Th epitopes contained within these sequences. Therefore, functional immunological Th analogues include several versions of the Th epitope derived from Measles Virus Fusion protein MvF1-4 Ths (SEQ ID NOs: 14, 21, 22-24, 31-33, 53-57, and 58-60) and from Hepatitis Surface protein HBsAg 1-3 Ths (SEQ ID NOs: 25-30, 34-36, 38, and 62-64).

### Compositions

The present disclosure also provides compositions comprising the disclosed PCSK9 immunogen peptide constructs.

a. Peptide Compositions

Compositions containing the disclosed PCSK9 peptide immunogen constructs can be in liquid or solid/lyophilized form. Liquid compositions can include water, buffers, solvents, salts, and/or any other acceptable reagent that does not alter the structural or functional properties of the PCSK9 peptide immunogen constructs. Peptide compositions can contain one or more of the disclosed PCSK9 peptide immunogen constructs.

b. Pharmaceutical Compositions

The present disclosure is also directed to pharmaceutical compositions containing the disclosed PCSK9 peptide immunogen constructs.

Pharmaceutical compositions can contain carriers and/or other additives in a pharmaceutically acceptable delivery system. Accordingly, pharmaceutical compositions can contain a pharmaceutically effective amount of a PCSK9 peptide immunogen construct together with pharmaceutically-acceptable carrier, adjuvant, and/or other excipients such as diluents, additives, stabilizing agents, preservatives, solubilizing agents, buffers, and the like.

Pharmaceutical compositions can contain one or more adjuvant that act(s) to accelerate, prolong, or enhance the immune response to the PCSK9 peptide immunogen constructs without having any specific antigenic effect itself. Adjuvants used in the pharmaceutical composition can include oils, oil emulsions, aluminum salts, calcium salts, immune stimulating complexes, bacterial and viral derivatives, virosomes, carbohydrates, cytokines, polymeric microparticles. In certain embodiments, the adjuvant can be selected from alum (potassium aluminum phosphate), aluminum phosphate (e.g. ADJU-PHOS®), aluminum hydroxide (e.g. ALHYDROGEL®), calcium phosphate, incomplete Freund's adjuvant (IFA), Freund's complete adjuvant, MF59, adjuvant 65, Lipovant, ISCOM, liposyn, saponin, squalene, L121, EMULSIGEN®, monophosphoryl lipid A (MPL), Quil A, QS21, MONTANIDE® ISA 35, ISA 50V, ISA 50V2, ISA 51, ISA 206, ISA 720, liposomes, phospholipids, peptidoglycan, lipopolysaccahrides (LPS), ASO1, ASO2, ASO3, ASO4, AF03, lipophilic phospholipid (lipid A), gamma inulin, algammulin, glucans, dextrans, glucomannans, galactomannans, levans, xylans, dimethyldioctadecylammonium bromide (DDA), as well as the other adjuvants and emulsifiers.

In some embodiments, the pharmaceutical composition contains MONTANIDE™ ISA 51 (an oil adjuvant composition comprised of vegetable oil and mannide oleate for production of water-in-oil emulsions), TWEEN® 80 (also known as: Polysorbate 80 or Polyoxyethylene (20) sorbitan monooleate), a CpG oligonucleotide, and/or any combination thereof. In other embodiments, the pharmaceutical composition is a water-in-oil-in-water (i.e. w/o/w) emulsion with EmulsIL-6n or EmulsIL-6n D as the adjuvant.

Pharmaceutical compositions can also include pharmaceutically acceptable additives or excipients. For example, pharmaceutical compositions can contain antioxidants, binders, buffers, bulking agents, carriers, chelating agents, coloring agents, diluents, disintegrants, emulsifying agents, fillers, gelling agents, pH buffering agents, preservatives, solubilizing agents, stabilizers, and the like.

Pharmaceutical compositions can be formulated as immediate release or for sustained release formulations. Additionally, the pharmaceutical compositions can be formulated for induction of systemic, or localized mucosal, immunity through immunogen entrapment and co-administration with microparticles. Such delivery systems are readily determined by one of ordinary skill in the art.

Pharmaceutical compositions can be prepared as injectables, either as liquid solutions or suspensions. Liquid vehicles containing the PCSK9 peptide immunogen construct can also be prepared prior to injection. The pharmaceutical composition can be administered by any suitable mode of application, for example, i.d., i.v., i.p., i.m., intranasally, orally, subcutaneously, etc. and in any suitable delivery device. In certain embodiments, the pharmaceutical composition is formulated for intravenous, subcutaneous, intradermal, or intramuscular administration. Pharmaceutical compositions suitable for other modes of administration can also be prepared, including oral and intranasal applications.

Pharmaceutical compositions can also be formulated in a suitable dosage unit form. In some embodiments, the pharmaceutical composition contains from about 0.1 μg to about 1 mg of the PCSK9 peptide immunogen construct per kg body weight. Effective doses of the pharmaceutical compositions vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. When delivered in multiple doses, the pharmaceutical compositions may be conveniently divided into an appropriate amount per dosage unit form. The administered dosage will depend on the age, weight and general health of the subject as is well known in the therapeutic arts.

In some embodiments, the pharmaceutical composition contains more than one PCSK9 peptide immunogen construct. A pharmaceutical composition containing a mixture of more than one PCSK9 peptide immunogen construct to allow for synergistic enhancement of the immunoefficacy of the constructs. Pharmaceutical compositions containing more than one PCSK9 peptide immunogen construct can be more effective in a larger genetic population due to a broad MHC class II coverage thus provide an improved immune response to the PCSK9 peptide immunogen constructs.

In some embodiments, the pharmaceutical composition contains a PCSK9 peptide immunogen construct selected from SEQ ID NOs: 65-107 (Table 3), as well as homologues, analogues, and/or combinations thereof.

In certain embodiments, PCSK9 peptide immunogen constructs (SEQ ID NOs: 91-93) with heterologous Th epitopes derived from MVF and HBsAg in a combinatorial form (SEQ ID NOs: 24, 30, and 36, respectively) were mixed in an equimolar ratio for use in a formulation to allow for maximal coverage of a host population having a diverse genetic background.

Furthermore, the antibody response elicited by the PCSK9 peptide immunogen constructs (e.g., utilizing UBITh®1; SEQ ID NO: 65) were mostly (>90%) focused on the desired cross-reactivity against the B cell epitope peptide of PCSK9 without much, if any, directed to the heterologous Th epitopes employed for immunogenicity enhancement. This is in sharp contrast to the conventional protein such as KLH or other biological protein carriers used for such PCSK9 peptide immunogenicity enhancement.

In other embodiments, pharmaceutical compositions comprising a peptide composition of for example a mixture of the PCSK9 peptide immunogen constructs in contact with mineral salts including Alum gel (ALHYDROGEL) or Aluminum phosphate (ADJUPHOS) as adjuvant to form a suspension formulation was used for administration to hosts.

Pharmaceutical compositions containing an PCSK9 peptide immunogen construct can be used to elicit an immune response and produce antibodies in a host upon administration.

c. Immunostimulatory Complexes

The present disclosure is also directed to pharmaceutical compositions containing a PCSK9 peptide immunogen construct in the form of an immunostimulatory complex with a CpG oligonucleotide. Such immunostimulatory complexes are specifically adapted to act as an adjuvant and/or as a peptide immunogen stabilizer. The immunostimulatory complexes are in the form of a particulate, which can efficiently present the PCSK9 peptide immunogen to the cells of the immune system to produce an immune response. The immunostimulatory complexes may be formulated as a suspension for parenteral administration. The immunostimulatory complexes may also be formulated in the form of water in oil (w/o) emulsions, as a suspension in combination with a mineral salt or with an in-situ gelling polymer for the efficient delivery of the PCSK9 peptide immunogen construct to the cells of the immune system of a host following parenteral administration.

The stabilized immunostimulatory complex can be formed by complexing a PCSK9 peptide immunogen construct with an anionic molecule, oligonucleotide, polynucleotide, or combinations thereof via electrostatic association. The stabilized immunostimulatory complex may be incorporated into a pharmaceutical composition as an immunogen delivery system.

In certain embodiments, the PCSK9 peptide immunogen construct is designed to contain a cationic portion that is positively charged at a pH in the range of 5.0 to 8.0. The net charge on the cationic portion of the PCSK9 peptide immunogen construct, or mixture of constructs, is calculated by assigning a +1 charge for each lysine (K), arginine (R) or histidine (H), a −1 charge for each aspartic acid (D) or glutamic acid (E) and a charge of 0 for the other amino acid within the sequence. The charges are summed within the cationic portion of the PCSK9 peptide immunogen construct and expressed as the net average charge. A suitable peptide immunogen has a cationic portion with a net average positive charge of +1. Preferably, the peptide immunogen has a net positive charge in the range that is larger than +2. In some embodiments, the cationic portion of the PCSK9 peptide immunogen construct is the heterologous spacer. In certain embodiments, the cationic portion of the PCSK9 peptide immunogen construct has a charge of +4 when the spacer sequence is (α, ε-N)Lys, (α,ε-N)-Lys-Lys-Lys-Lys (SEQ ID NO: 11), or Lys-Lys-Lys-ε-N-Lys (SEQ ID NO: 12).

An “anionic molecule” as described herein refers to any molecule that is negatively charged at a pH in the range of 5.0-8.0. In certain embodiments, the anionic molecule is an oligomer or polymer. The net negative charge on the oligomer or polymer is calculated by assigning a −1 charge for each phosphodiester or phosphorothioate group in the oligomer. A suitable anionic oligonucleotide is a single-stranded DNA molecule with 8 to 64 nucleotide bases, with the number of repeats of the CpG motif in the range of 1 to 10. Preferably, the CpG immunostimulatory single-stranded DNA molecules contain 18-48 nucleotide bases, with the number of repeats of CpG motif in the range of 3 to 8.

More preferably the anionic oligonucleotide is represented by the formula: 5′ X¹CGX² 3′ wherein C and G are unmethylated; and X¹ is selected from the group consisting of A (adenine), G (guanine) and T (thymine); and X² is C (cytosine) or T (thymine). Or, the anionic oligonucleotide is represented by the formula: 5′ (X³)₂CG(X⁴)₂ 3′ wherein C and G are unmethylated; and X³ is selected from the group consisting of A, T or G; and X⁴ is C or T. In specific embodiments, the CpG oligonucleotide has the sequence of CpG1: 5′ TCg TCg TTT TgT CgT TTT gTC gTT TTg TCg TT 3′ (fully phosphorothioated) (SEQ ID NO: 108), CpG2: 5′ Phosphate TCg TCg TTT TgT CgT TTT gTC gTT 3′ (fully phosphorothioated) (SEQ ID NO: 109), or CpG3 5′ TCg TCg TTT TgT CgT TTT gTC gTT 3′ (fully phosphorothioated) (SEQ ID NO: 110).

The resulting immunostimulatory complex is in the form of particles with a size typically in the range from 1-50 microns and is a function of many factors including the relative charge stoichiometry and molecular weight of the interacting species. The particulated immunostimulatory complex has the advantage of providing adjuvantation and upregulation of specific immune responses in vivo. Additionally, the stabilized immunostimulatory complex is suitable for preparing pharmaceutical compositions by various processes including water-in-oil emulsions, mineral salt suspensions and polymeric gels.

The present disclosure is also directed to pharmaceutical compositions, including formulations, for the prevention and/or treatment of patients with PCSK9 mediated disorders including an increased serum level of low-density lipoprotein cholesterol (LDL-C) and CV events. In some embodiments, pharmaceutical compositions comprising a stabilized immunostimulatory complex, which is formed through mixing a CpG oligomer with a peptide composition containing a mixture of the PCSK9 peptide immunogen constructs (e.g., SEQ ID NOs: 65-107) through electrostatic association, to further enhance the immunogenicity of the PCSK9 peptide immunogen constructs and elicit antibodies that are cross-reactive with the full-length PCSK9 protein of SEQ ID NO: 1 that are directed at the PCSK9/LDL-R receptor binding region.

In yet other embodiments, pharmaceutical compositions contain a mixture of the PCSK9 peptide immunogen constructs (e.g., any combination of SEQ ID NOs: 65-107) in the form of a stabilized immunostimulatory complex with CpG oligomers that are, optionally, mixed with mineral salts, including Alum gel (ALHYDROGEL) or Aluminum phosphate (ADJUPHOS) as an adjuvant with high safety factor, to form a suspension formulation for administration to hosts.

### Antibodies

The present disclosure also provides antibodies elicited by the PCSK9 peptide immunogen constructs.

The present disclosure provides PCSK9 peptide immunogen constructs and formulations thereof, cost effective in manufacturing, optimal in their design that are capable of eliciting high titer antibodies targeting the catalytic domain of the PCSK9 molecule (e.g., SEQ ID NOs: 2-9) and, more specifically, the PCSK9 and LDL-R receptor binding region (e.g., SEQ ID NOs: 2-6 and 8-9), that is capable of breaking the immune tolerance against self-protein PCSK9 with a high responder rate in immunized hosts. The antibodies generated by the PCSK9 peptide immunogen constructs have high affinity towards the PCSK9/LDL-R receptor binding region.

In some embodiments, PCSK9 peptide immunogen constructs for eliciting antibodies comprise a hybrid of a PCSK9 peptides targeting the catalytic domain of PCSK9, including the LDL-R receptor binding region, (e.g., SEQ ID NOs: 2-9) linked to a heterologous Th epitope derived from pathogenic proteins such as Measles Virus Fusion (MVF) protein and others (SEQ ID NOs: 13-64) through an optional spacer. The B cell epitope and Th epitope peptide of the PCSK9 peptide immunogen constructs act together to stimulate the generation of highly specific antibodies cross-reactive with the catalytic domain (SEQ ID NO: 111) of the full-length PCSK9 protein (SEQ ID NO: 1).

Traditional methods for immunopotentiating a peptide, such as through chemical coupling to a carrier protein, for example, Keyhole Limpet Hemocyanin (KLH) or other carrier proteins such as Diphtheria toxoid (DT) and Tetanus Toxoid (TT) proteins, typically result in the generation of a large amount of antibodies directed against the carrier protein. Thus, a major deficiency of such peptide-carrier protein compositions is that most (>90%) of antibodies generated by the immunogen are the non-functional antibodies directed against the carrier protein KLH, DT or TT, which can lead to epitopic suppression.

Unlike the traditional method for immunopotentiating a peptide, the antibodies generated by the disclosed PCSK9 peptide immunogen constructs (e.g. SEQ ID NOs: 65-107) bind with highly specificity to the catalytic domain of the PCSK9 B cell epitope peptide (SEQ ID NOs: 2-9) with little, if any, antibodies directed against the heterologous Th epitope (e.g., SEQ ID NOs: 13-64) or optional heterologous spacer.

Based on their unique characteristics and properties, the disclosed antibodies elicited by the PCSK9 peptide immunogen constructs are capable of providing a prophylactic immunotherapeutic approach to preventing and/or treating PCSK9 mediated disorders including an increased serum level of low-density lipoprotein cholesterol (LDL-C) and CV events in a subject.

### Methods

The present disclosure is also directed to methods for making and using the PCSK9 peptide immunogen constructs, compositions, and pharmaceutical compositions.

a. Methods for Manufacturing the PCSK9 Peptide Immunogen Construct

The PCSK9 peptide immunogen constructs of this disclosure can be made by chemical synthesis methods well known to the ordinarily skilled artisan (see, e.g., Fields, G. B., et al., 1992). The PCSK9 peptide immunogen constructs can be synthesized using the automated Merrifield techniques of solid phase synthesis with the α-NH₂ protected by either t-Boc or F-moc chemistry using side chain protected amino acids on, for example, an Applied Biosystems Peptide Synthesizer Model 430A or 431. Preparation of PCSK9 peptide immunogen constructs comprising combinatorial library peptides for Th epitopes can be accomplished by providing a mixture of alternative amino acids for coupling at a given variable position.

After complete assembly of the desired PCSK9 peptide immunogen construct, the resin can be treated according to standard procedures to cleave the peptide from the resin and the functional groups on the amino acid side chains can be deblocked. The free peptide can be purified by HPLC and characterized biochemically, for example, by amino acid analysis or by sequencing. Purification and characterization methods for peptides are well known to one of ordinary skill in the art.

The quality of peptides produced by this chemical process can be controlled and defined and, as a result, reproducibility of PCSK9 peptide immunogen constructs, immunogenicity, and yield can be assured. A detailed description of the manufacturing of the PCSK9 peptide immunogen construct through solid phase peptide synthesis is provided in Example 1.

The range in structural variability that allows for retention of an intended immunological activity has been found to be far more accommodating than the range in structural variability allowed for retention of a specific drug activity by a small molecule drug or the desired activities and undesired toxicities found in large molecules that are co-produced with biologically-derived drugs.

Thus, peptide analogues, either intentionally designed or inevitably produced by errors of the synthetic process as a mixture of deletion sequence byproducts that have chromatographic and immunologic properties similar to the intended peptide, are frequently as effective as a purified preparation of the desired peptide. Designed analogues and unintended analogue mixtures are effective as long as a discerning QC procedure is developed to monitor both the manufacturing process and the product evaluation process so as to guarantee the reproducibility and efficacy of the final product employing these peptides.

The PCSK9 peptide immunogen constructs can also be made using recombinant DNA technology including nucleic acid molecules, vectors, and/or host cells. As such, nucleic acid molecules encoding the PCSK9 peptide immunogen construct and immunologically functional analogues thereof are also encompassed by the present disclosure as part of the present invention. Similarly, vectors, including expression vectors, comprising nucleic acid molecules as well as host cells containing the vectors are also encompassed by the present disclosure as part of the present invention.

Various exemplary embodiments also encompass methods of producing the PCSK9 peptide immunogen construct and immunologically functional analogues thereof. For example, methods can include a step of incubating a host cell containing an expression vector containing a nucleic acid molecule encoding an PCSK9 peptide immunogen construct and/or immunologically functional analogue thereof under such conditions where the peptide and/or analogue is expressed. The longer synthetic peptide immunogens can be synthesized by well-known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made typically by synthesizing oligonucleotides which encode the peptide and any regulatory elements, if necessary. The synthetic gene is inserted in a suitable cloning vector and transfected into a host cell. The peptide is then expressed under suitable conditions appropriate for the selected expression system and host. The peptide is purified and characterized by standard methods.

b. Methods for the Manufacturing of Immunostimulatory Complexes

Various exemplary embodiments also encompass methods of producing the immunostimulatory complexes comprising PCSK9 peptide immunogen constructs and CpG oligodeoxynucleotide (ODN) molecule. Stabilized immunostimulatory complexes (ISC) are derived from a cationic portion of the PCSK9 peptide immunogen construct and a polyanionic CpG ODN molecule. The self-assembling system is driven by electrostatic neutralization of charge. Stoichiometry of the molar charge ratio of cationic portion of the PCSK9 peptide immunogen construct to anionic oligomer determines extent of association. The non-covalent electrostatic association of PCSK9 peptide immunogen construct and CpG ODN is a completely reproducible process. The peptide/CpG ODN immunostimulatory complex aggregates, which facilitate presentation to the “professional” antigen presenting cells (APC) of the immune system thus further enhancing the immunogenicity of the complexes. These complexes are easily characterized for quality control during manufacturing. The peptide/CpG ISC are well tolerated in vivo. This novel particulate system comprising CpG ODN and PCSK9 peptide immunogen constructs was designed to take advantage of the generalized B cell mitogenicity associated with CpG ODN use, yet promote balanced Th-1/Th-2 type responses.

The CpG ODN in the disclosed pharmaceutical compositions is 100% bound to immunogen in a process mediated by electrostatic neutralization of opposing charge, resulting in the formation of micron-sized particulates. The particulate form allows for a significantly reduced dosage of CpG from the conventional use of CpG adjuvants, less potential for adverse innate immune responses, and facilitates alternative immunogen processing pathways including antigen presenting cells (APC). Consequently, such formulations are novel conceptually and offer potential advantages by promoting the stimulation of immune responses by alternative mechanisms.

c. Methods for the Manufacturing of Pharmaceutical Compositions

Various exemplary embodiments also encompass pharmaceutical compositions containing PCSK9 peptide immunogen constructs. In certain embodiments, the pharmaceutical compositions employ water in oil emulsions and in suspension with mineral salts.

In order for a pharmaceutical composition to be used by a large population, safety becomes another important factor for consideration. Despite there has been use of water-in-oil emulsions in many clinical trials, Alum remains the major adjuvant for use in formulations due to its safety. Alum or its mineral salts Aluminum phosphate (ADJUPHOS) are, therefore, frequently used as adjuvants in preparation for clinical applications.

Other adjuvants and immunostimulating agents include 3 De-O-acylated monophosphoryl lipid A (MPL) or 3-DMP, polymeric or monomeric amino acids, such as polyglutamic acid or polylysine. Such adjuvants can be used with or without other specific immunostimulating agents, such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′ dipalmitoyl-sn-glycero-3-hydroxyphosphoryloxy)-ethyl amine (MTP-PE), N-acetylglucsaminyl-N-acetylmuramyl-L-Al-D-isoglu-L-Ala-dipalmitoxy propylamide (DTP-DPP) Theramide™), or other bacterial cell wall components. Oil-in-water emulsions include MF59 (see WO 90/14837 to Van Nest et al., which is hereby incorporated by reference in its entirety), containing 5% Squalene, 0.5% TWEEN 80, and 0.5% Span 85 (optionally containing various amounts of MTP-PE) formulated into submicron particles using a microfluidizer; SAF, containing 10% Squalene, 0.4% TWEEN 80, 5% pluronic-blocked polymer L121, and thr-MDP, either microfluidized into a submicron emulsion or vortexed to generate a larger particle size emulsion; and the Ribi™ adjuvant system (RAS) (Ribi ImmunoChem, Hamilton, Mont.) containing 2% squalene, 0.2% TWEEN 80, and one or more bacterial cell wall components selected from the group consisting of monophosphoryllipid A (MPL), trehalose dimycolate (TDM), and cell wall skeleton (CWS), preferably MPL+CWS (Detox™). Other adjuvants include Complete Freund's Adjuvant (CFA), Incomplete Freund's Adjuvant (IFA), and cytokines, such as interleukins (IL-1, IL-2, and IL-12), macrophage colony stimulating factor (M-CSF), and tumor necrosis factor (TNF-α).

The choice of an adjuvant depends on the stability of the immunogenic formulation containing the adjuvant, the route of administration, the dosing schedule, the efficacy of the adjuvant for the species being immunized, and, in humans, a pharmaceutically acceptable adjuvant is one that has been approved or is approvable for human administration by pertinent regulatory bodies. For example, alum, MPL or Incomplete Freund's adjuvant (Chang, J. C. C., et al., 1998), which is hereby incorporated by reference in its entirety) alone or optionally all combinations thereof are suitable for human administration.

The compositions can include pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, non-immunogenic stabilizers, and the like.

Pharmaceutical compositions can also include large, slowly metabolized macromolecules, such as proteins, polysaccharides like chitosan, polylactic acids, polyglycolic acids and copolymers (e.g., latex functionalized sepharose, agarose, cellulose, and the like), polymeric amino acids, amino acid copolymers, and lipid aggregates (e.g., oil droplets or liposomes). Additionally, these carriers can function as immunostimulating agents (i.e., adjuvants).

The pharmaceutical compositions of the present invention can further include a suitable delivery vehicle. Suitable delivery vehicles include, but are not limited to viruses, bacteria, biodegradable microspheres, microparticles, nanoparticles, liposomes, collagen minipellets, and cochleates.

d. Methods of Using Pharmaceutical Compositions

The present disclosure also includes methods of using pharmaceutical compositions containing PCSK9 peptide immunogen constructs.

In certain embodiments, the pharmaceutical compositions containing PCSK9 peptide immunogen constructs can be used for the treatment of patients with PCSK9 mediated disorders including an increased serum level of low-density lipoprotein cholesterol (LDL-C) and CV events.

In some embodiments, the methods comprise administering a pharmaceutical composition comprising a pharmacologically effective amount of a PCSK9 peptide immunogen construct to a host in need thereof. In certain embodiments, the methods comprise administering a pharmaceutical composition comprising a pharmacologically effective amount of an PCSK9 peptide immunogen construct to a warm-blooded animal (e.g., humans, Cynomolgus macaques, mice) to elicit highly specific antibodies cross-reactive with the full-length human PCSK9 protein (SEQ ID NO: 1).

In certain embodiments, the pharmaceutical compositions containing PCSK9 peptide immunogen constructs can be used to treat PCSK9 mediated disorders including an increased serum level of low-density lipoprotein cholesterol (LDL-C) and CV events in a subject.

e. In Vitro Functional Assays and In Vivo Proof of Concept Studies

Antibodies elicited in immunized hosts by the PCSK9 peptide immunogen constructs can be used in in vitro functional assays and in in vivo efficacy test. These functional assays or test include, but are not limited to:

a. generating highly specific antibodies capable of inhibiting PCSK9 and LDL-R receptor binding along with the associated downstream cellular events resulting in degradation of LDL-R, thus causing an increase in LDL-C uptake by LDL-R expressing cells; and

b. lowering levels of LDL-C and T-CHO in the plasma of the immunized host.

The disclosed PCSK9 peptide immunogen constructs and formulations thereof can effectively function as a pharmaceutical composition to prevent and/or treat subjects predisposed to, or suffering from, PCSK9 mediated disorders including an increased serum level of low-density lipoprotein cholesterol (LDL-C) and CV events in a subject.

### SPECIFIC EMBODIMENTS

(1) A PCSK9 peptide immunogen construct having about 20 or more amino acids, represented by the formulae:

(Th)ₘ-(A)ₙ-(PCSK9 functional B cell epitope peptide)-X

or

(PCSK9 functional B cell epitope peptide)-(A)ₙ-(Th)ₘ-X

or

(Th)ₘ-(A)ₙ-(PCSK9 functional B cell epitope peptide)-(A)ₙ-(Th)ₘ-X

wherein

Th is a heterologous T helper epitope;

A is a heterologous spacer;

(PCSK9 functional B cell epitope peptide) is a B cell epitope peptide having from 7 to about 30 amino acid residues derived from the catalytic domain of the PCSK9 protein (SEQ ID NO: 111);

X is an α-COOH or α-CONH₂ of an amino acid;

m is from 1 to about 4; and

n is from 0 to about 10.

(2) The PCSK9 peptide immunogen construct according to (1), wherein the PCSK9 functional B cell epitope peptide is selected from the group consisting of SEQ ID NOs: 2-9.
(3) The PCSK9 peptide immunogen construct according to (1), wherein the Th epitope is selected from the group consisting of SEQ ID NOs: 13-64.
(4) The PCSK9 peptide immunogen construct according to (1), wherein the PCSK9 functional B cell epitope peptide is selected from the group consisting of SEQ ID NOs: 2-9 and the Th epitope is selected from the group consisting of SEQ ID NOs: 13-64.
(5) The PCSK9 peptide immunogen construct according to (1), wherein the peptide immunogen construct is selected from the group consisting of SEQ ID NOs: 65-107.
(6) A PCSK9 peptide immunogen construct comprising:
a. a B cell epitope comprising from about 7 to about 30 amino acid residues from the catalytic domain of the PCSK9 sequence of SEQ ID NO: 111;
b. a T helper epitope comprising an amino acid sequence selected from the group consisting of SEQ ID NOs: 13-64, and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 11), Lys-Lys-Lys-ε-N-Lys (SEQ ID NO: 12), and Pro-Pro-Xaa-Pro-Xaa-Pro (SEQ ID NO: 10), and any combination thereof,
wherein the B cell epitope is covalently linked to the T helper epitope directly or through the optional heterologous spacer.
   (7) The PCSK9 peptide immunogen construct of (6), wherein the B cell epitope is selected from the group consisting of SEQ ID NOs: 2-9.
   (8) The PCSK9 peptide immunogen construct of (6), wherein the optional heterologous spacer is (α, ε-N)Lys, ε-N-Lys-Lys-Lys-Lys (SEQ ID NO: 11), Lys-Lys-Lys-ε-N-Lys (SEQ ID NO: 12), or Pro-Pro-Xaa-Pro-Xaa-Pro (SEQ ID NO: 10), where Xaa is any amino acid.
   (9) The PCSK9 peptide immunogen construct of (6), wherein the T helper epitope is covalently linked to the amino- or carboxyl-terminus of the B cell epitope.
   (10) The PCSK9 peptide immunogen construct of (6), wherein the T helper epitope is covalently linked to the amino- or carboxyl-terminus of the B cell epitope through the optional heterologous spacer.
   (11) A composition comprising the PCSK9 peptide immunogen construct according to (1).
   (12) A pharmaceutical composition comprising:
a. the PCSK9 peptide immunogen construct according to (1); and
b. a pharmaceutically acceptable delivery vehicle and/or adjuvant.
   (13) The pharmaceutical composition of (12), wherein
a. the PCSK9 functional B cell epitope peptide is selected from the group consisting of SEQ
b. the Th epitope is selected from the group consisting of SEQ ID NOs: 13-64; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 11), Lys-Lys-Lys-ε-N-Lys (SEQ ID NO: 12), and Pro-Pro-Xaa-Pro-Xaa-Pro (SEQ ID NO: 10), and any combination thereof; and
wherein the PCSK9 peptide immunogen construct is mixed with an CpG oligodeoxynucleotide (ODN) to form a stabilized immunostimulatory complex.
   (14) The pharmaceutical composition of (12), wherein
a. the PCSK9 peptide immunogen construct is selected from the group consisting of SEQ ID NOs: 65-107; and
wherein the PCSK9 peptide immunogen construct is mixed with an CpG oligodeoxynucleotide (ODN) to form a stabilized immunostimulatory complex.
   (15) A method for generating antibodies against PCSK9 in an animal comprising administering the pharmaceutical composition according to (12) to the animal.
   (16) An isolated antibody or epitope-binding fragment thereof that specifically binds to the PCSK9 and LDL-R receptor binding region of SEQ ID NOs: 2-9.
   (17) The isolated antibody or epitope-binding fragment thereof according to (16) bound to the PCSK9 peptide immunogen construct.
   (18) A composition comprising the isolated antibody or epitope-binding fragment thereof according to (16).
   (19) A method of preventing and/or treating patients with PCSK9 mediated disorders including an increased serum level of low-density lipoprotein cholesterol (LDL-C) and CV events in an animal comprising administering the pharmaceutical composition of (12) to the animal.

### Example 1

### Synthesis of PCSK9 Related Peptides and Preparation of Formulations Thereof

a. Synthesis of PCSK9 Related Peptides

Methods for synthesizing PCSK9 related peptides that were included in the development effort of PCSK9 peptide immunogen constructs are described. The peptides were synthesized in small-scale amounts that are useful for serological assays, laboratory pilot and field studies, as well as large-scale (kilogram) amounts, which are useful for industrial/commercial production of pharmaceutical compositions. A large repertoire of PCSK9 related antigenic peptides having sequences with lengths from approximately 10 to 70 amino acids were designed for epitope mapping and for the screening and selection of the most optimal peptide immunogen constructs for use in an efficacious PCSK9 targeted therapeutic vaccine.

Representative full-length human PCSK9 (SEQ ID NO: 1) and PCSK9 peptide fragments, are listed in Table 1 (SEQ ID NOs: 2-9).

Selected PCSK9 B cell epitope peptides were made into PCSK9 peptide immunogen constructs by synthetically linking to a carefully designed helper T cell (Th) epitope peptide derived from pathogen proteins, including Measles Virus Fusion protein (MVF), Hepatitis B Surface Antigen protein (HBsAg), influenza, Clostridium tetani, and Epstein-Barr virus (EBV) identified in Table 2 (SEQ ID NOs: 13-64). The Th epitope peptides were used either in a single sequence (13-23, 25-29, 31-32, 34-35, 37-56, 58-59, and 61-64) or a combinatorial library (SEQ ID NOs: 24, 30, 33, 36, 57, and 60) to enhance the immunogenicity of their respective PCSK9 peptide immunogen constructs.

Representative PCSK9 peptide immunogen constructs selected from hundreds of peptide constructs are identified in Table 3 (SEQ ID NOs: 65-107). All peptides used for immunogenicity studies or related serological tests for detection and/or measurement of anti-PCSK9 antibodies were synthesized on a small scale basis using F-moc chemistry by peptide synthesizers of Applied BioSystems Models 430A, 431 and/or 433. Each peptide was produced by an independent synthesis on a solid-phase support, with F-moc protection at the N-terminus and side chain protecting groups of trifunctional amino acids. Completed peptides were cleaved from the solid support and side chain protecting groups were removed by 90% Trifluoroacetic acid (TFA). Synthetic peptide preparations were evaluated by Matrix-Assisted Laser Desorption/Ionization-Time-Of-Flight (MALDI-TOF) Mass Spectrometry to ensure correct amino acid content. Each synthetic peptide was also evaluated by Reverse Phase HPLC (RP-HPLC) to confirm the synthesis profile and concentration of the preparation. Despite rigorous control of the synthesis process (including stepwise monitoring the coupling efficiency), peptide analogues were also produced due to unintended events during elongation cycles, including amino acid insertion, deletion, substitution, and premature termination. Thus, synthesized preparations typically included multiple peptide analogues along with the targeted peptide.

Despite the inclusion of such unintended peptide analogues, the resulting synthesized peptide preparations were nevertheless suitable for use in immunological applications including immunodiagnosis (as antibody capture antigens) and pharmaceutical compositions (as peptide immunogens). Typically, such peptide analogues, either intentionally designed or generated through synthetic process as a mixture of byproducts, are frequently as effective as a purified preparation of the desired peptide, as long as a discerning QC procedure is developed to monitor both the manufacturing process and the product evaluation process to guarantee the reproducibility and efficacy of the final product employing these peptides. Large scale peptide syntheses in the multi-hundred to kilo gram quantities can be conducted on a customized automated peptide synthesizer UBI2003 or the like at 15 mmole to 150 mmole scale.

For active ingredients used in the final pharmaceutical composition for clinical trials, PCSK9 peptide immunogen constructs were purified by preparative RP-HPLC under a shallow elution gradient and characterized by MALDI-TOF mass spectrometry, amino acid analysis, and RP-HPLC for purity and identity.

b. Preparation of Compositions Containing PCSK9 Peptide Immunogen Constructs

Formulations employing water-in-oil emulsions and in suspension with mineral salts were prepared. In order for a pharmaceutical composition designed to be used by a large population, safety becomes another important factor for consideration. Despite the fact that water-in-oil emulsions have been used in humans as pharmaceutical compositions in many clinical trials, Alum remains the major adjuvant for use in pharmaceutical composition due to its safety. Alum or its mineral salts ADJUPHOS (Aluminum phosphate) are therefore frequently used as adjuvants in preparation for clinical applications.

Briefly, the formulations specified in each of the study groups described below generally contained all types of designer PCSK9 peptide immunogen constructs. Over 40 peptide immunogen constructs were carefully evaluated in guinea pigs for their relative immunogenicity against the corresponding PCSK9 peptide used as the B cell epitope within the construct.

The PCSK9 peptide immunogen constructs at varying amounts were prepared in a water-in-oil emulsion with Seppic MONTANIDE™ ISA 51 as the approved oil for human use, or mixed with mineral salts ADJUPHOS (Aluminum phosphate) or ALHYDROGEL (Alum) as specified. Compositions were typically prepared by dissolving the PCSK9 peptide immunogen constructs in water at about 20 to 2,000 μg/mL and formulated with MONTANIDE™ ISA 51 into water-in-oil emulsions (1:1 in volume) or with mineral salts ADJUPHOS or ALHYDROGEL (Alum) (1:1 in volume). The compositions were kept at room temperature for about 30 min and mixed by vortex for about 10 to 15 seconds prior to immunization. Animals were immunized with 2 to 3 doses of a specific composition, which were administered at time 0 (prime) and 3 weeks post initial immunization (wpi) (boost), optionally 5 or 6 wpi for a second boost, by intramuscular route. Sera from the immunized animals were then tested with selected B cell epitope peptide(s) to evaluate the immunogenicity of the various PCSK9 peptide immunogen constructs present in the formulation and for the corresponding sera's cross-reactivity with PCSK9 proteins. Those PCSK9 peptide immunogen constructs with potent immunogenicity found in the initial screening in guinea pigs were further tested in in vitro assays for their corresponding sera's functional properties. The selected candidate PCSK9 peptide immunogen constructs were then prepared in water-in-oil emulsion, mineral salts, and alum-based formulations for dosing regimens over a specified period as dictated by the immunization protocols.

Only the most promising PCSK9 peptide immunogen constructs were further assessed extensively prior to being incorporated into final formulations for immunogenicity, duration, toxicity and efficacy studies in GLP guided preclinical studies in preparation for submission of an Investigational New Drug application followed by clinical trials in patients suffering from PCSK9 mediated disorders including an increased serum level of low-density lipoprotein cholesterol (LDL-C) and CV events.

The following examples serve to illustrate the present invention and are not to be used to limit the scope of the invention.

### Example 2

### Serological Assays and Reagents

Serological assays and reagents for evaluating functional immunogenicity of the PCSK9 peptide immunogen constructs and formulations thereof are described in detail below.

a. PCSK9 or PCSK9 B Cell Epitope Peptide-Based ELISA Tests for Immunogenicity and Antibody Specificity Analysis

ELISA assays for evaluating immune serum samples described in the following Examples were developed and described below. The wells of 96-well plates were coated individually for 1 hour at 37° C. with 100 μL of PCSK9 or PCSK9 B cell epitope peptides (e.g., SEQ ID NOs: 2-9), at 2 μg/mL (unless noted otherwise), in 10 mM NaHCO₃ buffer, pH 9.5 (unless noted otherwise).

The PCSK9 or PCSK9 B cell epitope peptide-coated wells were incubated with 250 μL of 3% by weight gelatin in PBS at 37° C. for 1 hour to block non-specific protein binding sites, followed by three washes with PBS containing 0.05% by volume TWEEN® 20 and dried. Sera to be analyzed were diluted 1:20 (unless noted otherwise) with PBS containing 20% by volume normal goat serum, 1% by weight gelatin and 0.05% by volume TWEEN® 20. One hundred microliters (100 μL) of the diluted specimens (e.g., serum, plasma) were added to each of the wells and allowed to react for 60 minutes at 37° C. The wells were then washed six times with 0.05% by volume TWEEN® 20 in PBS in order to remove unbound antibodies. Horseradish peroxidase (HRP)-conjugated species (e.g., guinea pig or rat) specific goat polyclonal anti-IgG antibody or Protein A/G were used as a labeled tracer to bind with the antibody/peptide antigen complex formed in positive wells. One hundred microliters (100 μL) of the HRP-labeled detection reagent, at a pre-titered optimal dilution and in 1% by volume normal goat serum with 0.05% by volume TWEEN® 20 in PBS, was added to each well and incubated at 37° C. for another 30 minutes. The wells were washed six times with 0.05% by volume TWEEN® 20 in PBS to remove unbound antibody and reacted with 100 μL of the substrate mixture containing 0.04% by weight 3′, 3′, 5′, 5′-Tetramethylbenzidine (TMB) and 0.12% by volume hydrogen peroxide in sodium citrate buffer for another 15 minutes. This substrate mixture was used to detect the peroxidase label by forming a colored product. Reactions were stopped by the addition of 100 μL of 1.0M H₂SO₄ and absorbance at 450 nm (A₄₅₀) determined. For the determination of antibody titers of the vaccinated animals that received the various peptide vaccine formulations, a 10-fold serial dilutions of sera from 1:100 to 1:10,000 or a 4-fold serial dilutions of sera from 1:100 to 1:4.19×10⁸ were tested, and the titer of a tested serum, expressed as Log₁₀, was calculated by linear regression analysis of the A₄₅₀ with the cutoff A₄₅₀ set at 0.5.

b. Assessment of Antibody Reactivity Towards Th Peptide by Th Peptide-Based ELISA Tests

The wells of 96-well ELISA plates were coated individually for 1 hour at 37° C. with 100 μL of Th peptide at 2 μg/mL (unless noted otherwise), in 10 mM NaHCO₃ buffer, pH 9.5 (unless noted otherwise) in similar ELISA method and performed as described above. For the determination of antibody titers of the vaccinated animals that received the various PCSK9 peptide vaccine formulations, 10-fold serial dilutions of sera from 1:100 to 1:10,000 were tested, and the titer of a tested serum, expressed as Log₁₀, was calculated by linear regression analysis of the A₄₅₀ with the cutoff A₄₅₀ set at 0.5.

c. Fine Specificity Analyses of a Target PCSK9 B Cell Epitope Peptide Determined by Epitope Mapping Through B Cell Epitope Cluster 10-Mer Peptide-Based ELISA Tests

Fine specificity analyses of anti-PCSK9 antibodies from hosts immunized with PCSK9 peptide immunogen constructs can be determined by epitope mapping using B cell epitope cluster 10mer peptide-based ELISA tests. Briefly, the wells of 96-well plates can be coated with individual PCSK9 10-mer peptides at 0.5 μg per 0.1 mL per well and then 100 μL serum samples (1:100 dilution in PBS) can be incubated in 10-mer plate wells in duplicate following the steps of the antibody ELISA method described above. The target B cell epitope specificity analyses of anti-PCSK9 antibodies from immunized hosts can be tested with corresponding PCSK9 peptide, or with non-relevant control peptide for specificity confirmation.

d. Immunogenicity Evaluation

Preimmune and immune serum samples from animal or human subjects were collected according to experimental vaccination protocols and heated at 56° C. for 30 minutes to inactivate serum complement factors. Following the administration of the vaccine formulations, blood samples were obtained according to protocols and their immunogenicity against specific target site(s) were evaluated by corresponding PCSK9 B cell epitope peptide-based ELISA tests. Serially diluted sera were tested and positive titers were expressed as Log₁₀ of the reciprocal dilution. Immunogenicity of a particular vaccine formulation is assessed for its ability to elicit high titer antibody response directed against the desired epitope specificity within the target antigen and high cross-reactivities with PCSK9 proteins, while maintaining a low to negligible antibody reactivity towards the T helper cell epitopes employed to provide enhancement of the desired B cell responses.

### Example 3

### Methods for Assessment of Functional Properties of Antibodies in an In Vitro Assay for LDL-C Uptake and Measurement of In Vivo Serum/Plasma Levels of LDL-C and T-CHO

Immune sera or purified anti-PCSK9 antibodies in immunized vaccines were further tested for their ability to suppress the PCSK9 binding to LDL-R receptor represented by the resulting LDL-C uptake by LDL-R expressing cell line.

a. Antibody Purification

All antibody purification procedures were followed according to manual of antibody purification kit (Thermo fisher, Cat no. 89953). The respective concentrations of IgG purification for each of the groups were carefully calibrated for use in in vitro assay.

b. Cell Preparation and Maintenance

HepG2 cell line was purchased from the American Type Culture Collection (Manassas, Va.) and maintained in DMEM medium supplemented with 10% Fetal Bovine Serum (FBS), 4.5 g/L L-glutamine, sodium pyruvate, and 1% penicillin/streptomycin in a humidified 37° C. incubator with 5% CO₂.

c. Cell Based LDL-C Uptake Assay

Human HepG2 cells were cultured in black, clear bottom 96-well microplate at a concentration of 50,000 cells per well in DMEM medium which was supplemented with 10% FBS. The cells were incubated at 37° C. for 48 hours. Thereafter, cells were starved with 0.3% BSA DMEM overnight. To form the PCSK9 and antibody-PCSK9 immune complex by purified antibodies from guinea pig preimmune or immune sera, 5 μg/mL of human PCSK9 (Biolegend, #592506) was incubated with varying concentrations of purified guinea pig polyclonal antibodies that were diluted serially in either uptake buffer (DMEM with 0.3% FBS) or in uptake buffer alone (control) for 1 hour at room temperature. After washing the cells with PBS, the PCSK9/antibody mixture was transferred to the cells in a 96 well plate, followed by LDL-BODIPY (Invitrogen) which was diluted in the uptake buffer at a final concentration of 5 μg/ml. After incubation for 2 hours at 37° C., cells were washed thoroughly with PBS and the cell fluorescence signal was detected by SpectraMax i3x reader (Molecular Devices) at 480-520 nm (excitation) and 520-600 nm (emission) as a parameter for measurement of LDL-C uptake by the Human HepG2 cells.

d. Measurement of Serum/Plasma LDL-C and T-CHO Levels in Guinea Pies

Plasma LDL-Cholesterol (LDL-C) and Total Cholesterol (T-CHO) levels in each animal and for each bleed were measured using Hitachi 7080 analyzer with Wako L-type CHO M kit (Cat #462-12491) and Roche L-type LDL-C(Cat #137520) respectively accordingly to manufacturer's instructions. Dilutions of cholesterol/LDL standards or test samples (70 μL per sample volume) were added to wells of a 96-well plate. One hundred forty microliters (40 μL) of prepared LDL-C reagents were added as calibrators. The plate was incubated for 5 minutes at 37° C. with the absorbance of the developed color being read at 600 nm within 30 minutes.

### Example 4

### Animals Used in Safety, Immunogenicity, Toxicity and Efficacy Studies

a. Guinea Pies

Immunogenicity studies were conducted in mature, naïve, adult male and female Duncan-Hartley guinea pigs (300-350 g/BW). The experiments utilized at least 3 guinea pigs per group. Protocols involving Duncan-Hartley guinea pigs (8-12 weeks of age; Covance Research Laboratories, Denver, Pa., USA) were performed under approved IACUC applications at a contracted animal facility under UBI sponsorship.

b. Immunization of Guinea Pies with Formulations Containing Placebo and PCSK9 Peptide Immunogen Constructs

Guinea pigs were used for the immunization, with 3 animals in each group. Animals in the experimental groups were respectively immunized with the PSCK9 peptide immunogen constructs formulated with ISA 51 and CpG at 400 μg/1.0 mL dose for prime and boost immunizations under intramuscular route. A total of five doses were generally administered at 0, 3, 6, 9, 12 and 15 WPI. All animals had free access to mice chow diet and water. The animals were bled generally at 0, 3, 6, 9, 12 and 15 WPI. Prior to bleeding, all animals were fasted for 12 hours. Blood samples were collected for measurement of titers against PCSK9 B cell epitope peptides or with full-length recombinant PCSK9 protein. Plasma LDL-C and T-CHO levels for each blood sample were also measured by standard blood testing procedure for component analysis.

### Example 5

### Vaccine Formulations for Immunogenicity Assessment of PCSK9 Peptide Constructs in Guinea Pigs

Pharmaceutical compositions and vaccine formulations used in each experiment are described in greater detail as shown below.

Briefly, the formulations specified in each of the study groups generally contained all types of designer PCSK9 peptide immunogen constructs with a segment of the PCSK9 B cell epitope peptide linked via different type of spacers (e.g., εLys (εK) or lysine-lysine-lysine (KKK)) to enhance the peptide construct's solubility and promiscuous helper T cell epitopes including two sets of artificial T helper epitopes derived from Measles virus fusion protein and Hepatitis B surface antigen. The PCSK9 B cell epitope peptides were linked at the N- or C-terminus of the designer peptide constructs. Several designer PCSK9 peptide immunogen constructs were initially evaluated in guinea pigs for their relative immunogenicity with the corresponding PCSK9 B cell epitope peptides. The PCSK9 peptide immunogen constructs were either prepared under varying amounts in a water-in-oil emulsion with Seppic MONTANIDE ISA 51 as the approved oil for human vaccine use, or with mineral salts (ADJUPHOS) or ALHYDROGEL (Alum) as a suspension, as specified. Formulations were usually prepared by dissolving the PCSK9 peptide constructs in water at about 20 to 800 μg/mL and formulated either with MONTANIDE ISA 51 into water-in-oil emulsions (1:1 in volume) or with mineral salts (ADJUPHOS) or ALHYDROGEL (Alum) (1:1 in volume). The formulations were kept at room temperature for about 30 min and mixed by vortex for about 10 to 15 seconds prior to immunization.

Some animals were immunized with 2 to 5 doses of a specific vaccine formulation, which were administered at time 0 (prime) and 3 weeks post initial immunization (wpi) (boost), optionally 5 or 6 wpi for a second boost, by intramuscular route. These immunized animals were then evaluated for the immunogenicity of the corresponding PCSK9 peptide immunogen constructs used in the respective formulations for their cross-reactivity with the corresponding PCSK9 B cell epitope peptides or full-length PCSK9. Those PCSK9 peptide immunogen constructs with potent immunogenicity in the initial screening in guinea pigs can be further tested in both water-in-oil emulsion, mineral salts, and alum-based formulations in other organisms for dosing regimens over a specified period as dictated by the immunization protocols.

### Example 6

### Design Rationale, Screening, Identification, Assessment of Functional Properties and Optimization of Multi-Component Vaccine Formulations Incorporating PCSK9 Peptide Immunogen Constructs for Treatment of Patients with PCSK9 Mediated Disorders Including an Increased Serum Level of Low-Density Lipoprotein Cholesterol (LDL-C) and CV Events

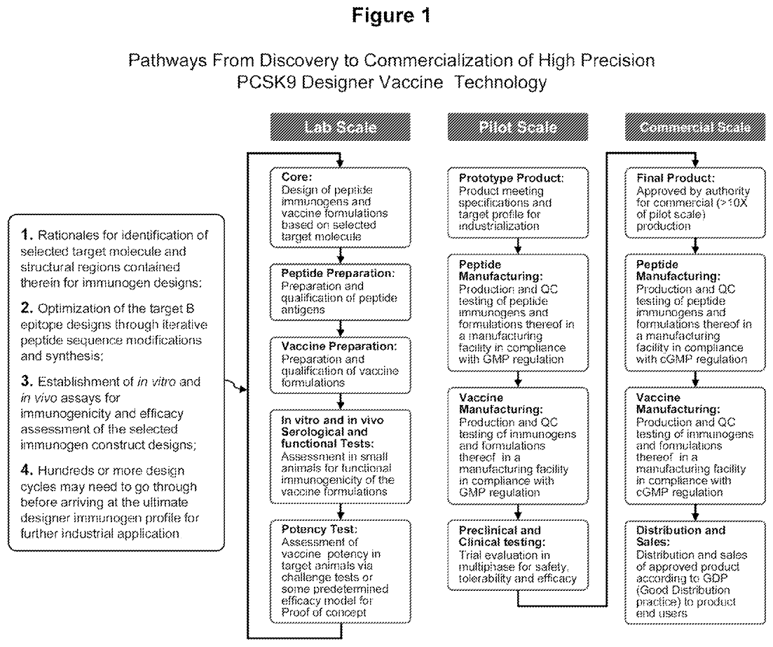

Based on scientific information provided by FIGS. 1 to 4, PCSK9 was selected as the target molecule for design of the disclosed peptide immunogen constructs. FIG. 1 depicts a general summary of the steps with a flow chart identifying the development process from discovery to commercialization (industrialization) of a PCSK9 vaccine formulation. Detailed evaluation and analyses of each of the steps has led to a myriad of experiments in the past that would ultimately result in commercialization of safe and efficacious pharmaceutical formulations containing PCSK9 peptide immunogen constructs.

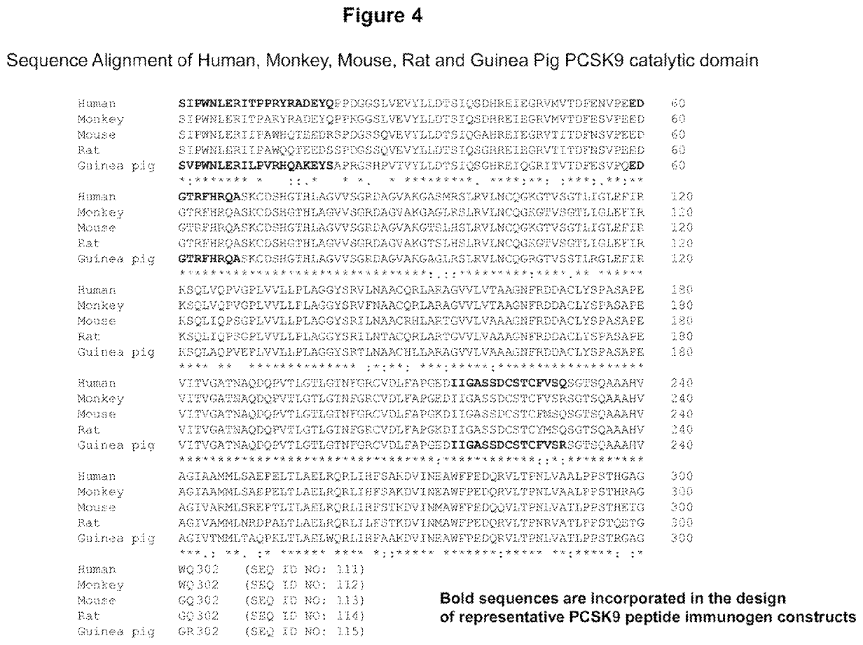

The mechanism and role of PCSK9 in LDL-C Metabolism is described in Chaudhary, R., et al., 2017. FIG. 2 depicts the full-length sequence of human PCSK9 (SEQ ID NO: 1) consisting of 692 amino acid residues. The full-length PCSK9 protein is composed of a signal peptide (residues 1-30), a prodomain (residues 31-152), a catalytic domain (residues 153-454), and a C-terminal (CT)-domain (455-692). Binding of PCSK9 to the EGF-A repeat of the LDL-R is mediated by a patch of residues in the catalytic domain of PCSK9 (SEQ ID NO: 111). The catalytic domain of PCSK9 is responsible both for autocatalytic cleavage and for binding of PCSK9 to the LDL-R. FIG. 3 identifies amino acid residues on PCSK9 and LDL-R that interface. These regions within the PCSK9 catalytic domain are where the disclosed PCSK9 peptide immunogen constructs of the current invention are designed around. FIG. 4 depicts sequence alignment of the catalytic domain of the PCSK9 from human, monkey, mouse, rat, and guinea pig. These alignments facilitate construct design to allow selection of analogue immunogen constructs to be tested in the target species to demonstrate the ability of the designer PCSK9 peptide immunogen constructs to break out immune tolerance.

a. Design History

Each peptide immunogen construct or immunotherapeutic product requires its own design focus and approach based on its specific disease mechanism and the target protein(s) required for intervention. For treatment of patients with PCSK9 mediated disorders, including an increased serum level of low-density lipoprotein cholesterol (LDL-C) and CV events, PCSK9 was selected as the target molecule based on the scientific information available, as outlined in FIGS. 1 to 4. The pathway from discovery to commercialization, as shown in FIG. 1, typically requires one or more decades to accomplish. Identification of the PCSK9 B cell epitope peptides correlating to the functional site(s) for intervention are key to the immunogen construct design. Consecutive pilot immunogenicity studies in guinea pigs incorporating various T helper support (carrier proteins or suitable T helper peptides) in various formulations were conducted and subsequently evaluated for the functional properties of the elicited purified antibodies or the vaccine formulations employing specific PCSK9 peptide immunogen constructs in specific in vitro functional assays or proof of concept in vivo studies in selected animal models. Upon extensive serological validation, candidate PCSK9 B cell epitope peptide immunogen constructs can be further tested in non-human primates to further validate the immunogenicity and direction of the PCSK9 peptide immunogen design. Selected PCSK9 peptide immunogen constructs can then be prepared in varying mixtures to evaluate the subtle differences in functional properties related to the respective interactions among peptide constructs when used in combinations. Upon additional evaluation, the final peptide constructs, peptide compositions, and pharmaceutical formulations thereof, along with the respective physical parameters of the formulations can be established leading to the final product development process.

The amino acid sequences of the PCSK9 peptide immunogen constructs were selected based on a number of design rationales. Several of these rationales include employing a PCSK9 B cell epitope peptide sequence that:

(i) is devoid of an autologous T helper epitope within PCSK9 to prevent autologous T cell activation;

(ii) is non-immunogenic on its own, since it is a self-molecule;

(iii) can be rendered immunogenic by a protein carrier or a potent T helper epitope(s) upon administration to a host:

(iv) elicits high titer antibodies directed against the PCSK9 peptide sequence (B cell epitope) and not against the protein carrier or potent T helper epitope(s);

(v) elicits high titer antibodies that would suppress/inhibit the PCSK9 binding to LDL-R on an LDL-R expressing cell line, thus allowing for the efficient LDL-C uptake by the LDL-R expressing cell line as measured by an in vitro LDL-C uptake assay; and

(vi) such vaccine formulations, when administered to an animal, would reduce the plasma/serum levels of LDL-C and T-CHO in vaccinated animals in a time-dependent manner.

b. Design and Validation of PCSK9 Peptide Immunogen Constructs for Pharmaceutical Compositions with Potential to Treat Patients Predisposed to, or Suffering from, PCSK9 Mediated Disorders, Including an Increased Serum Level of Low-Density Lipoprotein Cholesterol (LDL-C) and CV Events

In order to generate the most potent peptide constructs for incorporation into the pharmaceutical compositions, a repertoire of human PCSK9 B cell epitope peptides (e.g., SEQ ID NOs: 2-9) and promiscuous T helper epitopes derived from various pathogens or artificially T helper epitopes (e.g., SEQ ID NOs: 13-64) were designed. A representative number of PCSK9 peptide immunogen constructs (e.g., SEQ ID NOs: 65-107) were prepared for immunogenicity studies initially in guinea pigs.

i) Selection of PCSK9 B Cell Epitope Peptide Sequences from the Receptor Binding or Receptor Activation Region for Design

The PCSK9 and LDL-R receptor interface residues, as shown in FIG. 3, are located within the catalytic domain of the PCSK9 molecule with amino acid sequences around 153-172, 211-223, and 368-382, respectively. The catalytic domain of PCSK9 around these regions were selected for PCSK9 B cell epitope design and then further made into PCSK9 peptide immunogen constructs, as shown in Table 3. The peptide immunogen constructs were then used to elicit immune sera in guinea pigs for immunogenicity evaluation by ELISA on PCSK9 B cell epitope peptide coated plates and subsequently for in vitro functional assay assessment.

Under normal circumstance, LDL-C bound to the LDL-R is internalized into hepatocytes through clathrin-coated vesicles, after which the acidic environment of the endosome causes dissociation of LDL-C from its receptor. Recycling vesicles return the LDL-R to the cell surface, while endosomes containing the LDL-C particles fuse with lysosomes, resulting in degradation of LDL-C, hydrolysis of cholesterol esters, and distribution of free cholesterol to the rest of the cell. At the hepatocyte plasma membrane, the catalytic domain of secreted PCSK9 associates with the LDL-R and is internalized, entering the endosomal pathway. The low pH of the endosome enhances the affinity of PCSK9 for the LDL-R, preventing the receptor from being recycled to the cell surface. Instead, the complex is directed to the lysosome, where both components are degraded. In addition, PCSK9 appears to enhance intracellular LDL-R degradation prior to secretion, as PCSK9 can complex with the LDL-R within the Golgi and direct the receptor to the lysosome for degradation instead of transport to the plasma membrane.

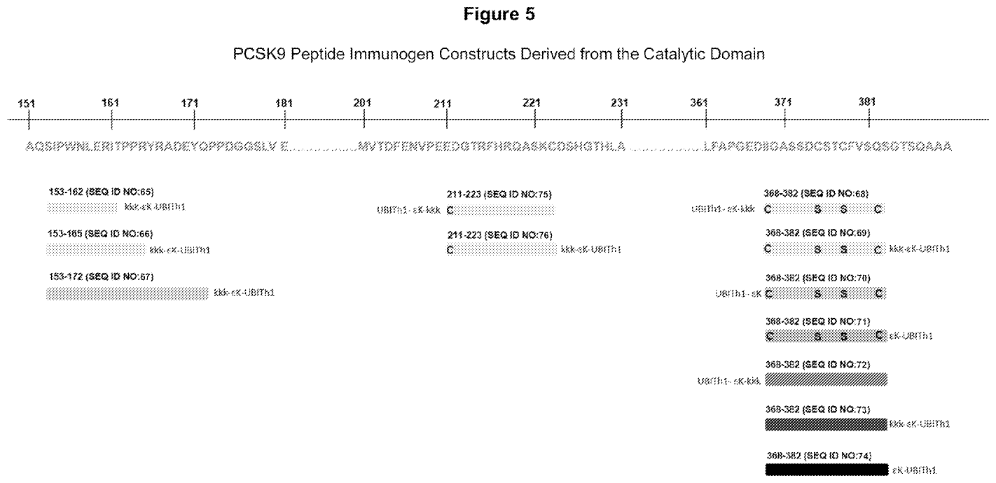

Representative PCSK9 B cell epitopes, such as those with SEQ ID NOs: 2 to 9 as shown in FIG. 5, and their counterpart PCSK9 peptide immunogen constructs (e.g., SEQ ID NOs: 65-76) were designed and synthesized. Formulations containing these PCSK9 peptide immunogen constructs were administered to guinea pigs for purpose of immunization and elicitation of potent polyclonal antibodies that target the PCSK9 B cell epitopes and are cross-reactive with the full-length PCSK9 protein.

Two cysteine residues are present within the PCSK9 B cell epitope region of amino acids 368-382 (SEQ ID NO: 5), which form a small four-member loop/ring constrained by the interaction of the Cys-Cys residues. A separate, modified form of this B cell epitope was designed for immunogenicity and functional testing. Specifically, in the modified form, the natural cysteine residues (at aa375 and aa378) were replaced with serine residues and the N-terminal isoleucine (at aa368) and the C-terminal glutamine (at aa382) were replaced with cysteine residues to produce the modified B cell epitope sequence of SEQ ID NO: 6. The modified B cell epitope sequence of SEQ ID NO: 6 formed a larger 15-member loop/ring structure constrained by the interaction of the Cys-Cys residues, which mimics the microenvironment around the C-terminal LDL-R receptor binding region.

For the PCSK9 B cell epitope region of amino acids 211-223, the glutamic acid (Glu) amino acid at amino acid position 211 was replaced with a cysteine residue. As a result, the modified B cell epitope sequence of SEQ ID NO: 7 forms a 13-member Cys-Cys loop/ring between the cysteine at aa211 and the natural cysteine at aa223, which mimics the microenvironment around the C-terminal LDL-R receptor binding region.

The PCSK9 peptide immunogen constructs containing SEQ ID NOs: 2-9 were formulated initially with ISA 51 and CpG for prime immunization in guinea pigs at 400 μg/1 mL and boosts (3, 6, and 9 wpi) at 100 μg/0.25 mL for immunogenicity studies.

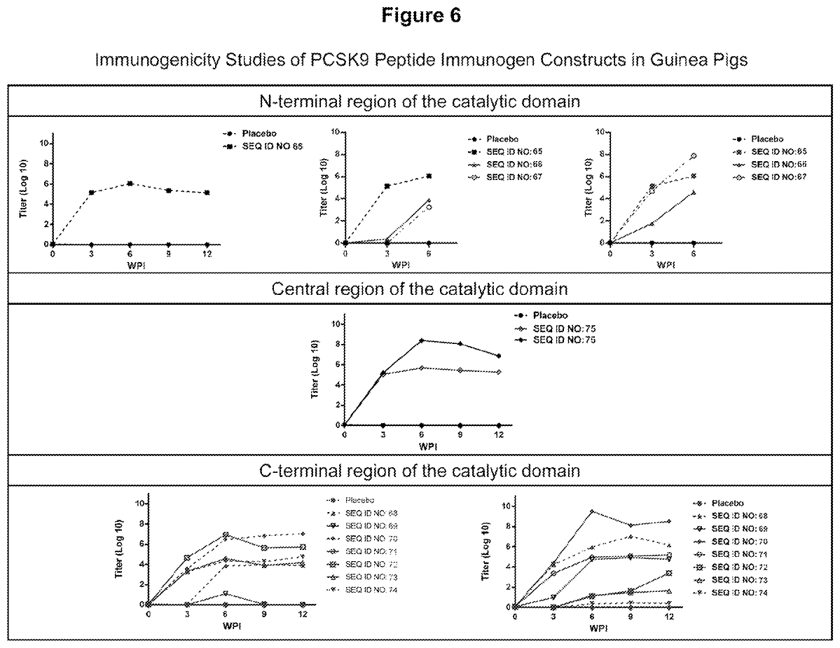

To test the immunogenicity in guinea pigs, an ELISA assay was used with guinea pig immune sera from various (wpi) bleeds, diluted at a 10-fold serial dilution from 1:100 to 1:10,000. ELISA plates were coated with corresponding human and guinea pig PCSK9 B cell epitope peptide and full-length PCSK9 protein at 0.5 μg peptide per well. The titer of a tested serum, expressed as Log₁₀, was calculated by linear regression analysis of the A450 nm with the cutoff A₄₅₀ set at 0.5 as shown in FIG. 6 for corresponding immunoreactivities with the PCSK9 B cell epitope peptides (SEQ ID NOs: 4, 5, 6 and 7) with detailed titers for representative B cell epitope derived PCSK9 peptide immunogen constructs as shown in Table 4 and FIG. 6. Despite that the designed short PCSK9 peptides frequently are non-immunogenic due to their lack of endogenous Th epitopes, addition of the foreign Th epitopes enhanced the immunogenicities of the specific PCSK9 peptide immunogen constructs.

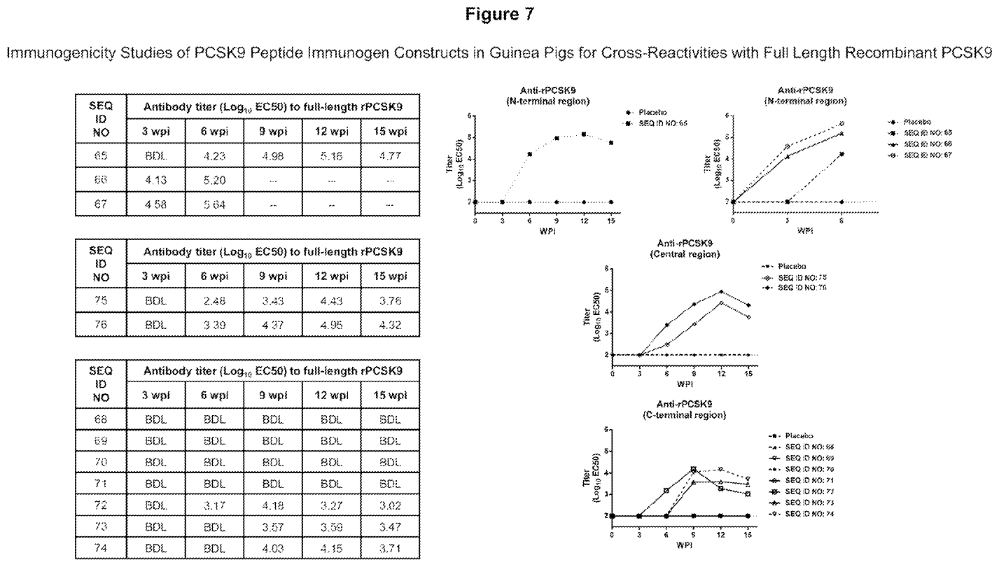

Analyses of the reactivity/specificity patterns of the various constructs reported in Table 4 and FIG. 6, can be further assessed to facilitate the design of optimal peptide immunogen constructs. Cross-reactivities with the full-length PCSK9 protein from guinea pig immune sera collected over the various time points were also assessed for their ability to function in a biological system. High cross-reactivities with the full-length recombinant human PCSK9 protein were found with most, if not all, at all of the time points, as shown in FIG. 7 along with antibody titers in Log₁₀ EC₅₀, as shown in the accompanying tables on the left panel of FIG. 7. Such high cross-reactivities found in these immune sera between the short PCSK9 B cell epitope peptides and that of a full-length PCSK9 protein demonstrate the high precision/fidelity nature of the immunogen design of the disclosed PCSK9 peptide immunogen constructs.

Table 5 provides a ranking of the binding efficiencies of the antibodies produced by the PCSK9 peptide immunogen constructs (SEQ ID NOs: 65 and 68-76) to the rPCSK9 protein based on the results shown in FIG. 7.

ii) Autologous T Helper Epitopes are not Present within the Selected PCSK9 B Cell Epitopes

The representative PCSK9 B cell epitopes tested, including those that contain SEQ ID NOs: 2 and 3, did not elicit any antibodies to PCSK9 (data not shown). These results demonstrate that the PCSK9 B cell epitopes described herein do not contain undesirable endogenous Th epitopes that are capable of eliciting immune responses on their own.

iii) The Antibody Response Elicited by PCSK9 Peptide Immunogen Constructs is Targeted to the PCSK9 B Cell Epitope Only, not the Th Epitope

It is well known that all carrier proteins (e.g. Keyhole Limpet Hemocyanin (KLH), Diphtheria toxoid (DT) and Tetanus Toxoid (TT) proteins) used to potentiate an immune response directed against the targeted B cell epitope peptide, by chemical conjugation of such B cell epitope peptide to the respective carrier protein, will elicit more than 90% of the antibodies directed against the potentiating carrier protein with less than 10% of the antibodies directed against the targeted B cell epitope in immunized hosts.

It is, therefore, of interest to assess the specificity of the PCSK9 peptide immunogen constructs of the present invention. One representative PCSK9 peptide immunogen construct (SEQ ID NO: 65), with the B cell epitope from human PCSK9 153-162 that is linked through a spacer sequence to the heterologous T cell epitope UBITh®1 (SEQ ID NO: 37), was prepared for immunogenicity assessment. The UBITh®1 (T helper peptide used for B cell epitope immunopotentiation) was coated on ELISA plates and the guinea pig immune sera was evaluated to test for cross-reactivities with the UBITh®1 peptide used for immunopotentiation. The results demonstrated that, in contrast to the high immunogenicity of these constructs towards the corresponding targeted PCSK9 B cell epitope peptides, the immune sera were found non-reactive to the UBITh®1 peptide (data not shown).

In summary, peptide immunogen design incorporating target PCSK9 B cell epitope peptide linked to carefully selected T helper epitope allows the generation of a focused immune response targeted only to the corresponding PCSK9 B cell epitope peptide. Based on the data obtained, it was found that pharmaceutical compositions that produce highly specific the immune responses directed toward the PCSK9 B cell epitope corresponds to a higher safety profile for the composition. The PCSK9 peptide immunogen constructs of the present disclosure is thus highly specific and highly potent against its B cell target.

iv) Fine Epitope Mapping with Immune Sera Directed Against Selected PCSK9 Peptide Immunogen Constructs can be Performed

A fine epitope mapping study to localize the antibody binding site(s) to specific residues within the target B cell epitope regions of PCSK9 can be performed by designing overlapping 10-mer peptides that cover PCSK9 amino acids 144-182, 201-233, 358-392, which cover the PCSK9 and LDL-R receptor binding regions of the catalytic domain of the PCSK9 molecule. These 10-mer peptides can be individually coated onto 96-well microtiter plate wells as solid-phase immune-absorbents. The pooled guinea pig antisera can be added at a 1:100 dilution in specimen diluent buffer to the plate wells coated with 10-mer peptide at 2.0 μg/mL followed by incubation for one hour at 37° C. After washing the plate wells with wash buffer, horseradish peroxidase-conjugated rProtein A/G can be added and incubated for 30 min. After washing with PBS again, the substrate can be added to the wells for measurement of absorbance at 450 nm by ELISA plate reader, when the samples are analyzed in duplicate. The binding of PCSK9 peptide immunogen elicited immune sera to the corresponding PCSK9 B cell epitope peptide coated wells would represent the maximal antibody binding signal.

The fine epitope mapping results would reveal whether the pooled guinea pig sera from PCSK9 peptide immunogen constructs comprising PCSK9 B cell epitope peptides from the N-terminal, central, and/or C-terminal regions of the catalytic region induces high titer antibodies with high cross-reactivities to recombinant human PCSK9 protein compared to other B cell epitope peptides.

In summary, the designed synthetic PCSK9 peptide immunogen constructs tested thus far induced robust immune responses in guinea pigs generating polyclonal antibodies targeted at distinct clusters B cell epitope peptides within the catalytic domain of the PCSK9 molecule. These regions are in close proximity to the PCSK9-LDL-R receptor binding regions near the respective N-terminal, central, and C-terminal regions of the catalytic domain of the PCSK9 molecule, allowing for important medical interventions. Epitope mapping along with functional assay assessment can be performed that would allow identification of the most optimal peptide immunogen constructs for use in pharmaceutical compositions containing PCSK9 peptide immunogen constructs.

### Example 7

### Assessment of Functional Properties of Antibodies by Measurement of In Vivo Serum/Plasma Levels of LDL-C and T-CHO in Hosts Immunized with PCSK9 Peptide Immunogen Constructs and Formulations Thereof

After demonstration of the high immunogenicity and cross-reactivities of the immune sera of guinea pigs immunized with carefully selected candidate PCSK9 immunogen constructs as shown in Table 4, FIGS. 6 and 7, the following studies were designed to assess whether the serum/plasma levels of LDL-C and T-CHO were affected as a result of immunizations in guinea pigs where close sequence identity of the sequences between guinea pigs and humans are present.

Measurement of In Vivo Serum/Plasma Levels of LDL-C and T-CHO in Guinea Pigs Immunized with PCSK9 Peptide Immunogen Constructs

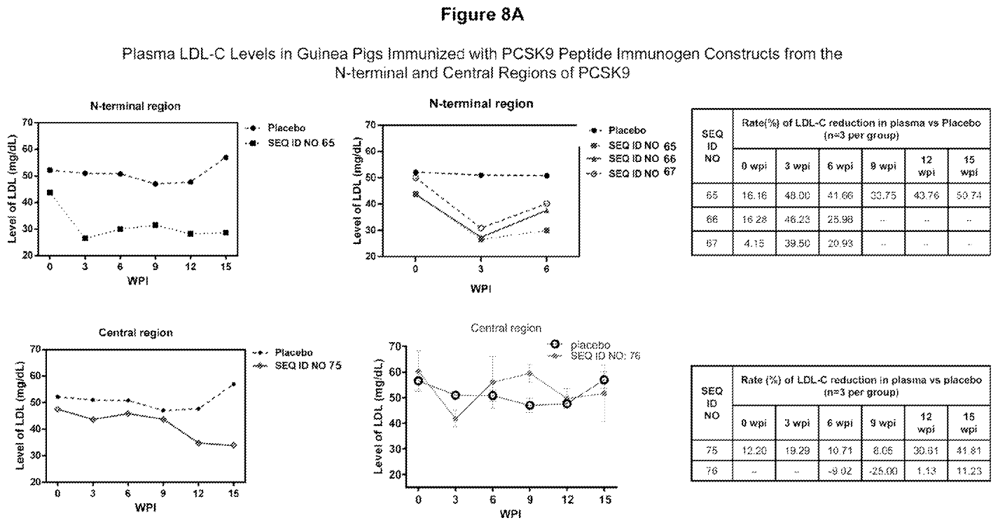

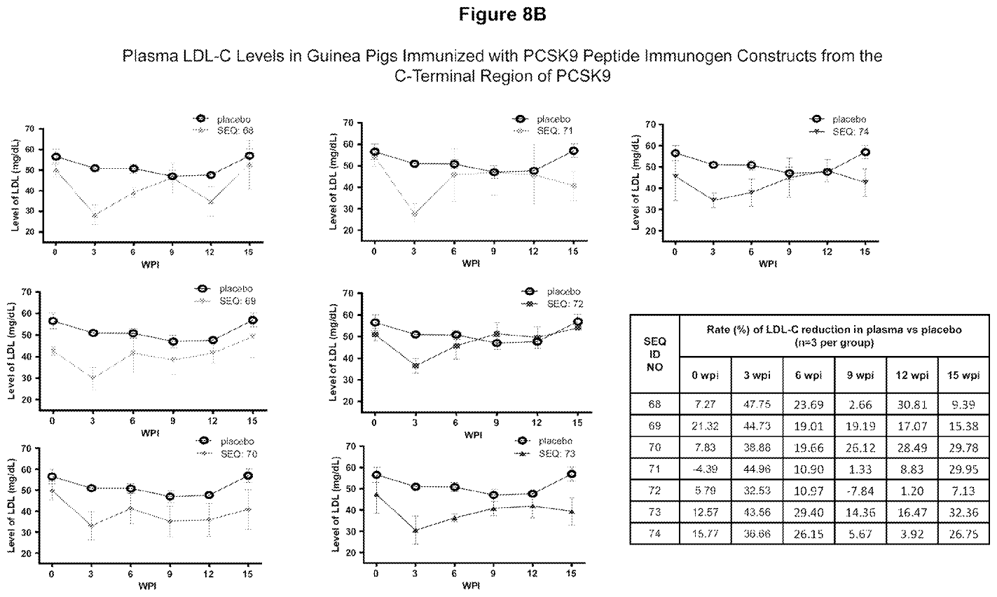

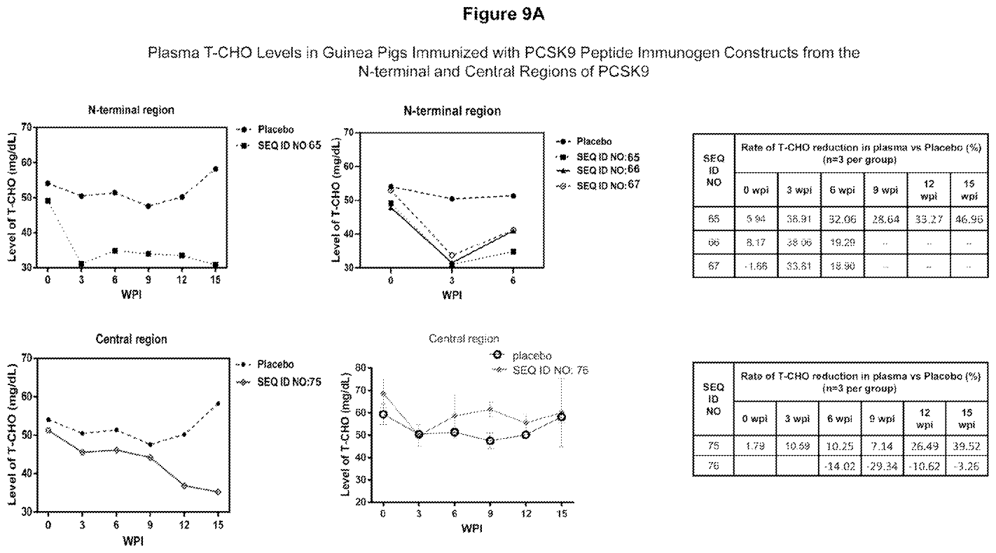

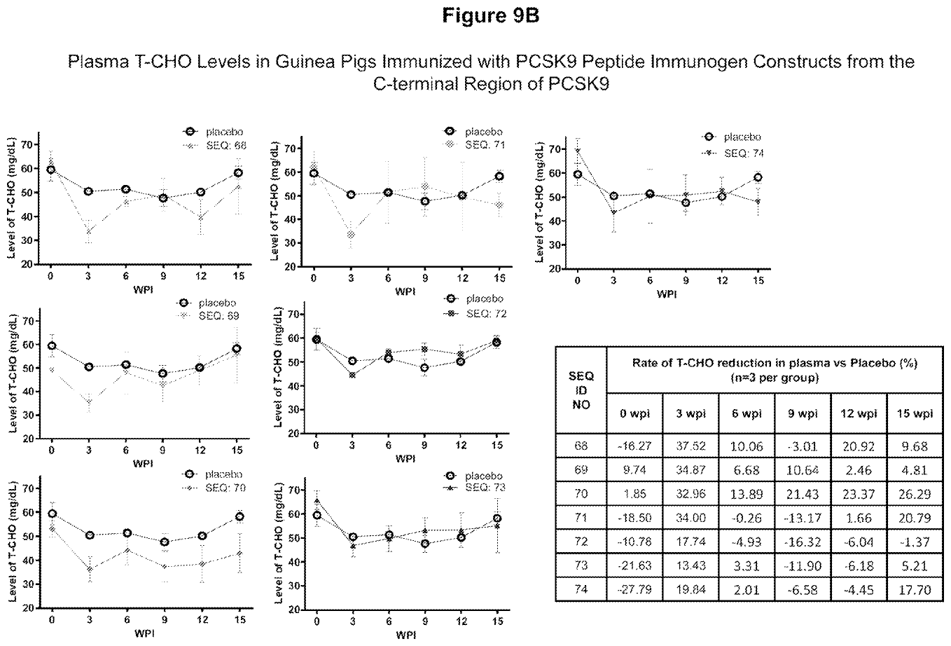

As described in Example 3 above, serum/plasma levels of LDL-Cholesterol (LDL-C) and Total Cholesterol (T-CHO) in each animal and for each bleed were measured using Hitachi 7080 analyzer with Wako L-type CHO M kit (Cat #462-12491) and Roche L-type LDL-C(Cat #137520), respectively, accordingly to the manufacturer's instructions. Dilutions of T-CHO/LDL-C standards or test samples (70 μL per sample volume) were added to wells of a 96-well plate. 140 μL of prepared LDL-C reagents were added as calibrators. The plate was incubated for 5 minutes at 37° C. with the absorbance of the developed color being read at 600 nm within 30 minutes. FIGS. 8A-8B and 9A-9B show the levels of LDL-C and T-CHO, respectively, measured in guinea pigs immunized with PCSK9 peptide immunogen constructs with PCSK9 B cell epitopes derived from N-terminal, central and C-terminal regions within the catalytic domain of PCSK9 (SEQ ID NOs: 65, 66, 68, 75, and 68-74). Significant reduction in serum/plasma LDL-C was found for the constructs with B cell epitopes derived from both the N-terminal and C-terminal regions at as early as 3 weeks from initial immunization at week 0, as shown on the left panels of FIGS. 8A and 8B. Such reduction is around 20-50% when compared to the basal level of LDL-C measured at week 0. All LDL-C levels were further compared to the levels of those in the placebo group. The overall rate of reduction when compared to those in animals of the placebo group for each time point was in the range of 30 to 50% of reduction. For the LDL-C levels from animals immunized with the PCSK9 immunogen construct of SEQ ID NO: 75, with B cell epitope derived from the central region of the catalytic domain of PCSK9 (SEQ ID NO: 7), a delayed reduction was observed in the first 9 weeks despite a reasonable antibody cross-reactivity with the full-length recombinant PCSK9 protein. The rate of LDL-C reduction then returned to a level in par with those from the N-terminal and C-terminal regions of the PCSK9 B cell epitopes. As shown in FIGS. 9A and 9B, a parallel trend of reduction in serum/plasma levels for T-CHO was observed for all serum/plasma samples collected at all time points measured.

In summary, for all animals immunized with the specially designed PCSK9 peptide immunogen constructs, immediate and significant reduction of serum/plasma levels of LDL-C and T-CHO was observed, which demonstrates that the PCSK9 peptide immunogen constructs of the present disclosure and formulations containing the constructs have in vivo proof of efficacy. These results demonstrate that the PCSK9 peptide immunogen constructs are capable of breaking out of immune tolerance with the generation of site-specific antibodies towards a very important self-protein to allow modulation of the LDL-C uptake by cells such as hepatocytes expressing LDL-R to enhance its LDL-C and T-CHO clearance rate from the blood serum/plasma.

Table 5 provides a ranking of the T-CHO inhibition rate (%) and LDL inhibition rate (%) produced by antibodies elicited from the PCSK9 peptide immunogen constructs (SEQ ID NOs: 65 and 68-76) based on the results shown in FIGS. 8A-8B and 9A-9B.

### Example 8

### Assessment of Functional Properties of Antibodies from Hosts Immunized with PCSK9 Peptide Immunogen Constructs for their Ability to Enhance LDL-C Uptake in an In Vitro Assay

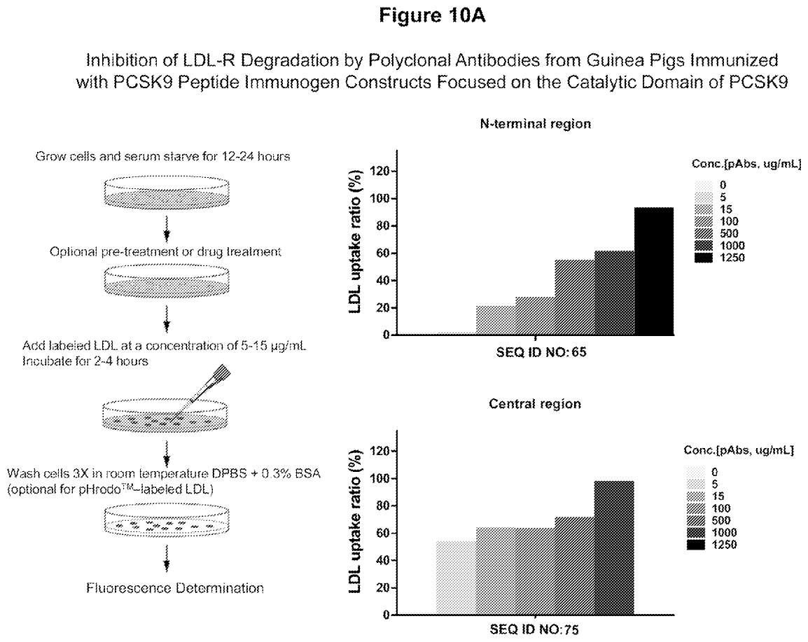

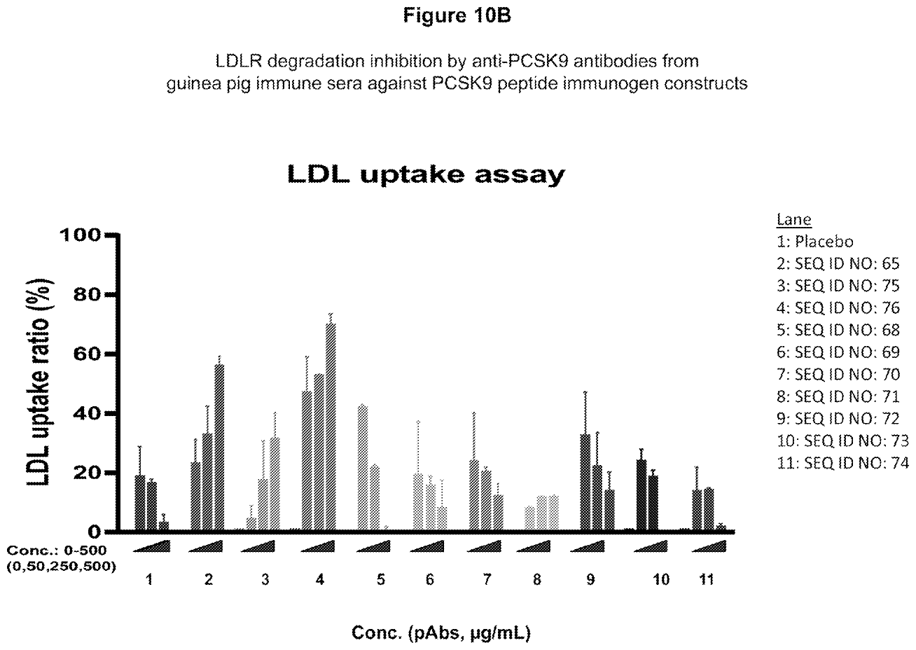

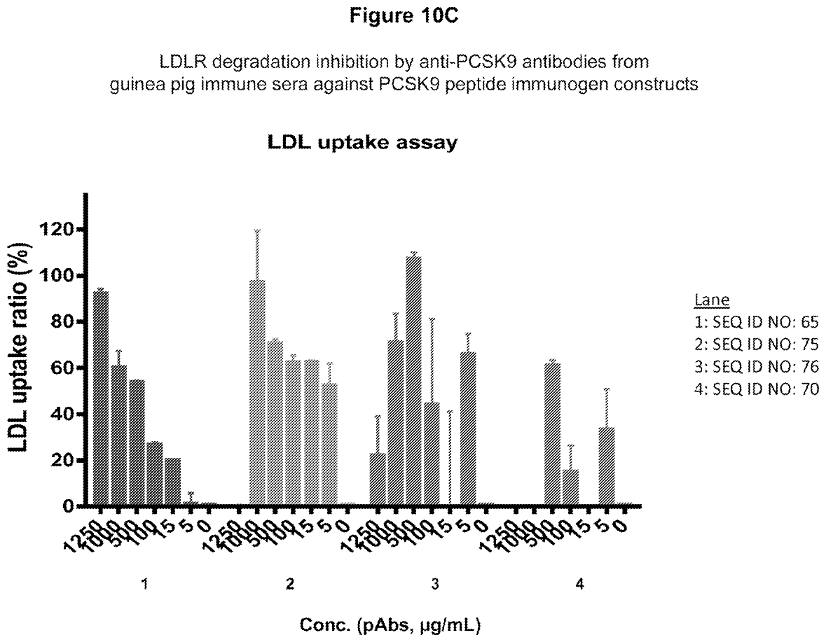

The functional properties of antibodies from hosts immunized with the disclosed PCSK9 peptide immunogen constructs and formulations thereof were assessed by an in vitro assay as described in Example 3 for LDL-C uptake using hepatocytes expressing LDL-R as the system for such measurement. FIG. 10A illustrates the results of a representative study by assessing the capability of the polyclonal antibodies purified from 12 wpi immune sera of guinea pigs immunized with PCSK9 peptide immunogen constructs (e.g., SEQ ID NOs: 65 and 75). The rationale for such a study design is that anti-PCSK9 antibodies elicited by these peptide immunogen constructs would inhibit and prevent PCSK9 binding to LDL-R. This inhibition would prevent the internalization of the PCSK9-LDL-R complex and the following intracellular events leading to degradation of the LDL-R. A lower surface expression of LDL-R through recyclization of receptor usage would reduce its uptake of LDL-C from the serum/plasma. The LDL update assay procedure is shown on the left panel of FIG. 10A and the outcome of LDL uptake ratio (indicative of LDL-R expression on the surface without being degraded due to PCSK9 binding) is shown as bar graphs on the right panel. As can be seen from FIG. 10A, an enhanced LDL-C uptake is observed with purified antibodies from both PCSK9 peptide immunogen constructs with SEQ ID NOs: 65 and 75 in an antibody dose (0, 5, 15, 100, 500, 1,000, 1,250 μg/mL) dependent fashion.

A similar study was performed, using the assay procedure shown in FIG. 10A, with polyclonal antibodies purified from 12 wpi immune sera of guinea pigs immunized with peptide immunogen constructs from the N-terminal region (SEQ ID NOs: 65), the Central region (SEQ ID NOs: 75-76), and the C-terminal region (SEQ ID NOs: 68-74) of PCSK9 with antibody doses ranging from 0, 50, 250, and 500 μg/mL, as shown in FIG. 10B. FIG. 10B shows that an enhanced LDL-C uptake was observed with purified antibodies from SEQ ID NOs: 65 and 75 in an antibody dose dependent fashion, which is consistent with the results shown in FIG. 10A. FIG. 10B also shows the PCSK9 peptide immunogen construct of SEQ ID NOs: 71 and 76 also produced an enhanced LDL-C uptake in a dose dependent fashion, with SEQ ID NO: 75 producing a significantly stronger response compared to SEQ ID NO: 71. Interestingly, the PCSK9 peptide immunogen construct of SEQ ID NOs: 68-70 and 72-74 all produced an enhanced LDL-C uptake with an antibody dose at 50 μg/mL, but that enhancement was reduced at higher concentrations of the antibody (250 and 500 μg/mL).

A third study was performed, using the assay procedure shown in FIG. 10A, with polyclonal antibodies purified from 15 wpi immune sera of guinea pigs immunized with peptide immunogen constructs from the N-terminal region (SEQ ID NOs: 65), the Central region (SEQ ID NOs: 75-76), and the C-terminal region (SEQ ID NO: 70) of PCSK9 with antibody doses ranging from 1,250, 1,000, 500, 100, 15, 5, and 0 μg/mL, as shown in FIG. 10C. FIG. 10C shows that an enhanced LDL-C uptake was observed with purified antibodies from SEQ ID NOs: 65 and 75 in an antibody dose dependent fashion, which is consistent with the results shown in FIGS. 10A and 10B. The results for SEQ ID NO: 75 show that the level of enhanced LDL-C uptake was relatively consistent over the antibody dose range of 5 to 1,000 μg/mL. The results for SEQ ID NO: 76 demonstrate that the antibody dose of 500 μg/mL produce the strongest LDL-C uptake compared to the other doses. Finally, the results for SEQ ID NO: 70 demonstrate that the 5 and 500 μg/mL antibody doses produced the strongest LDL-C uptake compared to the other doses tested.

In summary, purified antibodies derived from immune sera of 12 wpi of guinea pigs immunized with representative PCSK9 peptide immunogen constructs of SEQ ID NOs: 65, 75, 76, and 68-74 and immune sera from 15 wpi of guinea pigs immunized with SEQ ID NOs: 65, 75, 76, and 70 derived from the PCSK9 and LDL-R binding regions would enhance LDL-R uptake in cells expressing LDL-R leading to better clearance of LDL-C from serum/plasma in blood, thus demonstrating an important functional property of the anti-PCSK9 polyclonal antibodies leading to in vivo efficacy.

Table 5 provides a ranking of the LDL uptake ratio (%) produced by antibodies elicited from the PCSK9 peptide immunogen constructs (SEQ ID NOs: 65 and 68-76) based on the results shown in FIGS. 10A-10C.

### Example 9

### Assessment of the Immunogenicities and Functional Properties of Antibodies Directed Against the N-Terminal Region of PCSK9

The immunogenicity of two additional PCSK9 peptide immunogen constructs of SEQ ID NOs: 66 and 67, containing the B cell epitopes of SEQ ID NOs: 3 and 4, respectively, were evaluated according to the protocol described in Example 6.

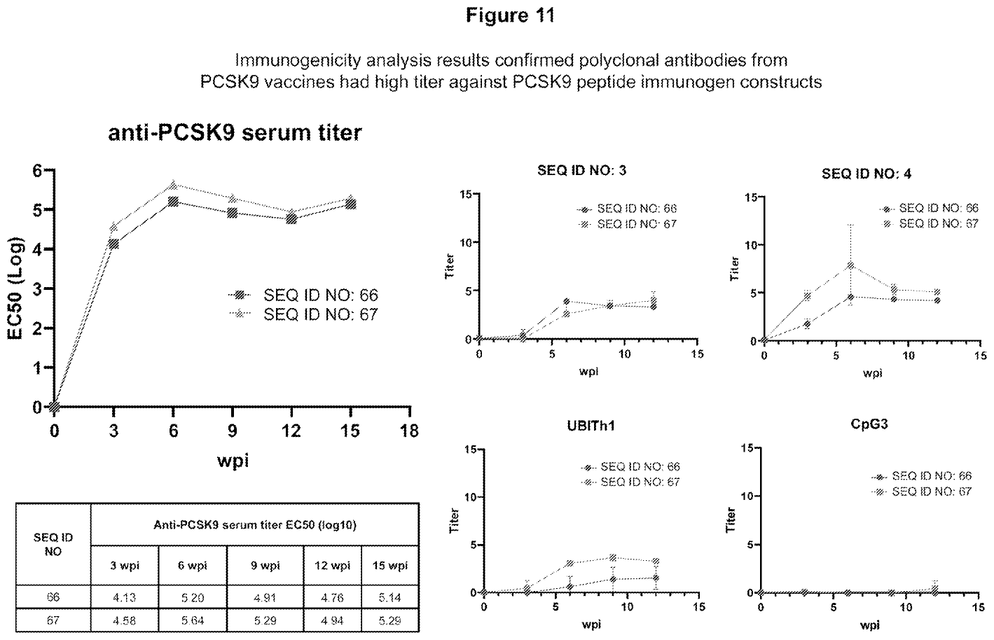

The left panel of FIG. 11 shows that guinea pigs immunized with PCSK9 peptide immunogens of SEQ ID NO: 66 or 67 produced high immunogenicity titers, beginning as early as 3 wpi and those titers were maintained through 15 wpi.

The graphs shown in the right panel of FIG. 11 demonstrate that the antibodies elicited by the peptide immunogen constructs are highly reactive against the B cell epitopes of PCSK9 (SEQ ID NOs: 3 and 4) and not against the Th epitope of UBITh®1 or the CpG3 oligonucleotide.

In addition, the serum T-CHO and LDL-C levels were evaluated in animals immunized with the PCSK9 peptide immunogen constructs of SEQ ID NOs: 66 and 67. Specifically, guinea pigs were immunized with the PCSK9 peptide immunogens according to the protocol described in Table 6.

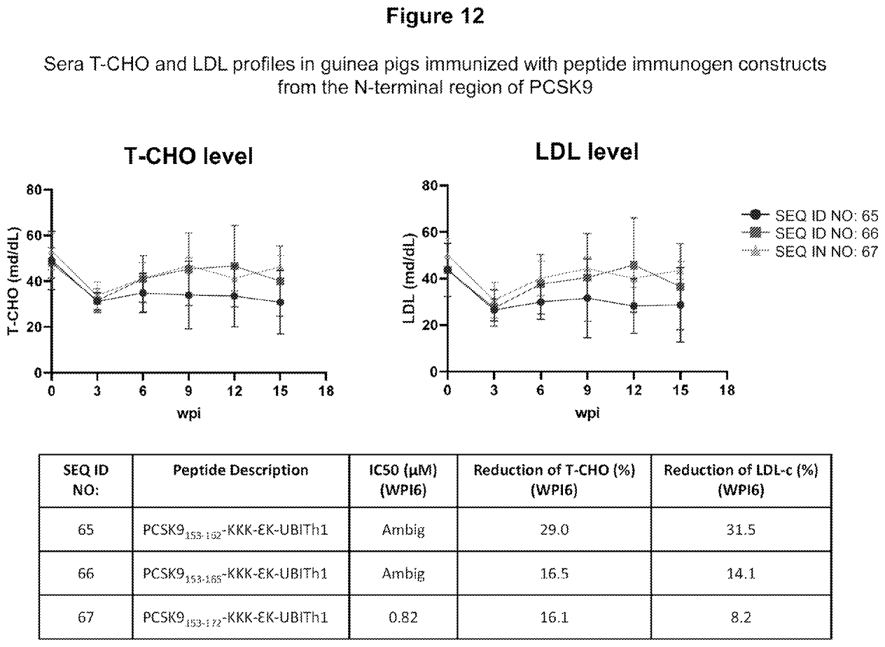

The results from this study are shown in FIG. 12, with the results from SEQ ID NO: 65 (shown in FIGS. 8A and 9A) included for comparison. Specifically, FIG. 12 shows that the peptide immunogen constructs from the N-terminal region of PCSK9 (SEQ ID NOs: 65-67) were effective in reducing the T-CHO and LDL levels in immunized guinea pigs. The T-CHO and LDL reduction levels were comparable between SEQ ID NOs: 66-67 throughout the study and the T-CHO and LDL levels were reduced to a greater extent in guinea pigs immunized with SEQ ID NO: 65 compared to SEQ ID NOs: 66 and 67.

TABLE 1
Amino Acid Sequences of PCSK9 and Fragments
Thereof Employed in Serological Assays

| | SEQ | |
|---|---|---|
| Amino Acid | ID | |
| positions | NO: | Sequence |
| Human PCSK9 | 1 | MGTVS SRRSW WPLPL LLLLL LLLGP |
| (full- | | AGARA QEDED GDYEE LVLAL RSEED |
| length) | | GLAEA PEHGT TATFH RCAKD PWRLP |
| | | GTYVV VLKEE THLSQ SERTA RRLQA |
| | | QAARR GYLTK ILHVF HGLLP GFLVK |
| | | MSGDL LELAL KLPHV DYIEE DSSVF |
| | | AQSIP WNLER ITPPR YRADE YQPPD |
| | | GGSLV EVYLL DTSIQ SDHRE IEGRV |
| | | MVTDF ENVPE EDGTR FHRQA SKCDS |
| | | HGTHL AGVVS GRDAG VAKGA SMRSL |
| | | RVLNC QGKGT VSGTL IGLEF IRKSQ |
| | | LVQPV GPLVV LLPLA GGYSR VLNAA |
| | | CQRLA RAGVV LVTAA GNFRD DACLY |
| | | SPASA PEVIT VGATN AQDQP VTLGT |
| | | LGTNF GRCVD LFAPG EDIIG ASSDC |
| | | STCFV SQSGT SQAAA HVAGI AAMML |
| | | SAEPE LTLAE LRQRL IHFSA KDVIN |
| | | EAWFP EDQRV LTPNL VAALP PSTHG |
| | | AGWQL FCRTV WSAHS GPTRM ATAIA |
| | | RCAPD EELLS CSSFS RSGKR RGERM |
| | | EAQGG KLVCR AHNAF GGEGV YAIAR |
| | | CCLLP QANCS VHTAP PAEAS MGTRV |
| | | HCHQQ GHVLT GCSSH WEVED LGTHK |
| | | PPVLR PRGQP NQCVG HREAS IHASC |
| | | CHAPG LECKV KEHGI PAPQE QVTVA |
| | | CEEGW TLTGC SALPG TSHVL GAYAV |
| | | DNTCV VRSRD VSTTG STSEE AVTAV |
| | | SAICCR RHLA QASQE LQ |
| PCSK9 | 2 | SIPWN LERIT |
| 153-162 | | |
| PCSK9 | 3 | SIPWN LERIT PPR |
| 153-165 | | |
| PCSK9 | 4 | SIPWN LERIT PPRYR ADEYQ |
| 153-172 | | |
| PCSK9 | 5 | IIGAS SDCST CFVSQ |
| 368-382 | | |
| PCSK9 | 6 | CIGAS SDSST SFVSC |
| 368-382_ | | |
| mod | | |
| (I368→C368, | | |
| C375→S375, | | |
| C378→S378, | | |
| Q382→C382) | | |
| PCSK9 | 7 | CDGTR FHRQA SKC |
| 211-223 | | |
| Guinea Pig | 8 | SVPWN LERIL |
| PCSK9 | | |
| 153-162 | | |
| Monkey/ | 9 | IIGAS SDCST CFVSR |
| Guinea Pig | | |
| PCSK9 | | |
| 368-382 | | |
| Flexible | 10 | PPXPXP |
| Hinge | | |
| Spacer | | |
| Spacer1 | 11 | ϵK-KKK |
| Spacer2 | 12 | KKK-ϵK |

* Peptides are cyclized by cysteine disulfide bonds, as shown with the cysteines underlined.
The Cysteines/Serines that substitute the amino acids of the PCSK9 fragments are in italics.

TABLE 2
Amino Acid Sequences of Pathogen Protein
Derived Th Epitopes Including Idealized
Artificial Th Epitopes for Employment in
the Design of PCSK9 Peptide Immunogen
Constructs

| | SEQ | |
|---|---|---|
| | ID | |
| Description | NO: | Sequence |
| Clostridium | 13 | KKQYIKANSKFIGITEL |
| tetani1 Th | | |
| (UBITh ® 6) | | |
| MvF1 Th | 14 | LSEIKGVIVHRLEGV |
| (UBITh ® 5) | | |
| Bordetella | 15 | GAYARCPNGTRALTVAELRGNAEL |
| pertussis Th | | |
| (UBITh ® 7) | | |
| Clostridium | 16 | WVRDIIDDFTNESSQKT |
| tetani2 Th | | |
| Diphtheria | 17 | DSETADNLEKTVAALSILPGHGC |
| Th | | |
| Plasmodium | 18 | DHEKKHAKMEKASSVFNVVNS |
| falciparum | | |
| Th | | |
| Schistosoma | 19 | KWFKTNAPNGVDEKHRH |
| mansoni Th | | |
| Cholera | 20 | ALNIWDRFDVFCTLGATTGYLKGNS |
| Toxin Th | | |
| MvF2 Th | 21 | ISEIKGVIVHKIEGI |
| KKKMvF3 Th | 22 | KKKISISEIKGVIVHKIEGILF |
| | 23 | T RT TR T |
| | 24 | KKKISIXEIXXVIVXXIEXILF |
| HBsAg1 Th | 25 | KKKLFLLTKLLTLPQSLD |
| | 26 | RRRIKII RII I L IR |
| | 27 | VRVV VV V I V |
| | 28 | F FF FF F V F |
| | 29 | F |
| | 30 | XXXXXXXTXXXTXPXSXX |
| MvF4 Th | 31 | ISISEIKGVIVHKIETILF |
| (UBITh ® 3) | 32 | T RT TR |
| | 33 | ISIXEIXXVIVXXIETILF |
| HBsAg2 Th | 34 | KKKIITITRIITIPQSLD |
| | 35 | FFLL L ITTI |
| | 36 | KKKXXXXTRIXTIXXXXD |
| MvF5 Th | 37 | ISITEIKGVIVHRIETILF |
| (UBITh ® 1) | | |
| HBsAg3 Th | 38 | KKKIITITRIITIITTID |
| (UBITh ® 2) | | |
| Influenza | 39 | FVFTLTVPSER |
| MP1_1 Th | | |
| Influenza | 40 | SGPLKAEIAQRLEDV |
| MP1_2 Th | | |
| Influenza | 41 | DRLRRDQKS |
| NSP1 Th | | |
| EBV | 42 | AGLTLSLLVICSYLFISRG |
| BHRF1 Th | | |
| Clostridium | 43 | QYIKANSKFIGITEL |
| tetani TT1 | | |
| Th | | |
| EBV EBNA-1 | 44 | PGPLRESIVCYFMVFLQTHI |
| Th | | |
| Clostridium | 45 | FNNFTVSFWLRVPKVSASHLE |
| tetani TT2 | | |
| Th | | |
| Clostridium | 46 | KFIIKRYTPNNEIDSF |
| tetani TT3 | | |
| Th | | |
| Clostridium | 47 | VSIDKFRIFCKALNPK |
| tetani TT4 | | |
| Th | | |
| EBV CP Th | 48 | VPGLYSPCRAFFNKEELL |
| HCMV IE1 | 49 | DKREMWMACIKELH |
| Th | | |
| EBV GP340 | 50 | TGHGARTSTEPTTDY |
| Th | | |
| EBV BPLF1 | 51 | KELKRQYEKKLRQ |
| Th | | |
| EBV EBNA-2 | 52 | TVFYNIPPMPL |
| Th | | |
| MvF Th | 53 | DLSDLKGLLLHKLDGL |
| (SSAL1 Th1) | 54 | EI EIR III RIE I |
| | 55 | V V VVV V V |
| | 56 | F F FFF F F |
| | 57 | XXSXXXGXXXHXXXGX |
| MvF3 Th | 58 | ISISEIKGVIVHKIEGILF |
| | 59 | T RT TR T |
| | 60 | ISIXEIXXVIVXXIEXILF |
| KKKMvF5 Th | 61 | KKKISITEIKGVIVHRIETILF |
| (UBITh ® 1a) | | |
| HBsAg Th | 62 | FFLLTRILTIPQSLD |
| (UBITh ® 4) | | |
| KKK-HBsAg | 63 | KKKFFLLTRILTIPQSLD |
| Th | | |
| HBsAg Th | 64 | FFLLTRILTIPQSL |

TABLE 3
Amino Acid Sequences of PCSK9 Peptide
Immunogen Constructs

| | SEQ | |
|---|---|---|
| Peptide | ID | |
| Description | NO: | Sequence |
| PCSK9 153-162- | 65 | SIPWNLERIT-KKK- |
| KKK-ϵK-UBITh1 | | ϵK-UBTH1 |
| PCSK9 153-165- | 66 | SIPWNLERITPPR- |
| KKK-ϵK-UBITh1 | | KKK-ϵK-UBTH1 |
| PCSK9 153-172- | 67 | SIPWNLERITPPRYRADEYQ- |
| KKK-ϵK-UBITh1 | | KKK-ϵK-UBTH1 |
| UBITh1-ϵK-KKK- | 68 | UBTh1-ϵK-KKK- |
| PCSK9 368-382_ | | CIGASSDSSTSFVSC |
| mod | | |
| PCSK9 368-382_ | 69 | CIGASSDSSTSFVSC-KKK- |
| mod-KKK-ϵK- | | ϵK-UBITh1 |
| UBITh1 | | |
| UBITh1-ϵK-PCSK9 | 70 | UBTh1-ϵK- |
| 368-382_mod | | CIGASSDSSTSFVSC |
| PCSK9 368-382_ | 71 | CIGASSDSSTSFVSC- |
| mod-ϵK-UBITh1 | | ϵK-UBITh1 |
| UBITh1-ϵK-KKK- | 72 | UBTh1-ϵK-KKK- |
| PCSK9 368-382 | | IIGASSDCSTCFVSQ |
| PCSK9 368-382- | 73 | IIGASSDCSTCFVSQ-KKK- |
| KKK-ϵK-UBITh1 | | ϵK-UBITh1 |
| PCSK9 368-382- | 74 | IIGASSDCSTCFVSQ-ϵK- |
| ϵK-UBITh1 | | UBITh1 |
| UBITh1-ϵK-KKK- | 75 | UBITh1-ϵK-KKK- |
| PCSK9 211-223 | | CDGTRFHRQASKC |
| PCSK9 211-223- | 76 | CDGTRFHRQASKC-KKK- |
| KKK-ϵK-UBITh1 | | ϵK-UBITh1 |
| Guinea Pig PCSK9 | 77 | SVPWNLERIL-KϵKK-K- |
| 153-162-KKK-ϵK- | | UBTH1 |
| UBITh1 | | |
| UBITh1-ϵK-KKK- | 78 | UBTh1-ϵK-KKK- |
| Guinea Pig PCSK9 | | IIGASSDCSTCFVSR |
| 368-382 | | |
| Guinea Pig PCSK9 | 79 | IIGASSDCSTCFVSR- |
| 368-382-KKK-ϵK- | | KKK-ϵK-UBITh1 |
| UBITh1 | | |
| PCSK9 153-162-KKK- | 80 | SIPWNLERIT-KKK-ϵK- |
| ϵK-UBITh2 | | KKKIITITRIITIITTID |
| PCSK9 153-162-KKK- | 81 | SIPWNLERIT-KKK-ϵK- |
| ϵK-UBITh3 | | ISISEIKGVIVHKIETILF |
| | | T RT TR |
| PCSK9 153-162-KKK- | 82 | SIPWNLERIT-KKK-ϵK- |
| ϵK-C. tetani1 Th | | KKQYIKANSKFIGITEL |
| PCSK9 153-162-KKK- | 83 | SIPWNLERIT-KKK-ϵK- |
| ϵK-MvF1 Th | | LSEIKGVIVHRLEGV |
| PCSK9 153-162-KKK- | 84 | SIPWNLERIT-KKK-ϵK- |
| ϵK-B. pertussis Th | | GAYARCPNGTRALTVAELRGNAEL |
| PCSK9 153-162-KKK- | 85 | SIPWNLERIT-KKK-ϵK- |
| ϵK-C. tetani2 Th | | WVRDIIDDFTNESSQKT |
| PCSK9 153-162-KKK- | 86 | SIPWNLERIT-KKK-ϵK- |
| ϵK-Diphtheria Th | | DSETADNLEKTVAALSILPGHGC |
| PCSK9 153-162-KKK- | 87 | SIPWNLERIT-KKK-ϵK- |
| ϵK-P. falciparum Th | | DHEKKHAKMEKASSVFNVVNS |
| PCSK9 153-162-KKK- | 88 | SIPWNLERIT-KKK-ϵK- |
| ϵK-S. mansoni Th | | KWFKTNAPNGVDEKHRH |
| PCSK9 153-162-KKK- | 89 | SIPWNLERIT-KKK-ϵK- |
| ϵK-Cholera Toxin Th | | ALNIWDRFDVFCTLGATTGYLKGNS |
| PCSK9 153-162-KKK- | 90 | SIPWNLERIT-KKK-ϵK- |
| ϵK-MvF2 Th | | ISEIKGVIVHKIEGI |
| PCSK9 153-162-KKK- | 91 | SIPWNLERIT-KKK-ϵK- |
| ϵK-KKKMvF3 Th | | KKKISISEIKGVIVHKIEGILF |
| | | T RT TR T |
| PCSK9 153-162-KKK- | 92 | SIPWNLERIT-KKK-ϵK- |
| ϵK-HBsAg1 Th | | KKKLFLLTKLLTLPQSLD |
| | | RRRIKII RII I L IR |
| | | VRVV VV V I V |
| | | F FF FF F V F |
| | | F |
| PCSK9 153-162-KKK- | 93 | SIPWNLERIT-KKK-ϵK- |
| ϵK-HBsAg2 Th | | KKKIITITRIITIPQSLD |
| | | FFLL L ITTI |
| PCSK9 153-162-KKK- | 94 | SIPWNLERIT-KKK-ϵK- |
| ϵK-Influenza MP1_1 | | FVFTLTVPSER |
| Th | | |
| PCSK9 153-162-KKK- | 95 | SIPWNLERIT-KKK-ϵK- |
| ϵK-Influenza MP1_2 | | SGPLKAEIAQRLEDV |
| Th | | |
| PCSK9 153-162-KKK- | 96 | SIPWNLERIT-KKK-ϵK- |
| ϵK-Influenza NSP1 | | DRLRRDQKS |
| Th | | |
| PCSK9 153-162-KKK- | 97 | SIPWNLERIT-KKK-ϵK- |
| ϵK-EBV BHRF1 Th | | AGLTLSLLVICSYLFISRG |
| PCSK9 153-162-KKK- | 98 | SIPWNLERIT-KKKϵ-K- |
| ϵK-C. tetani TT1 | | QYIKANSKFIGITEL |
| Th | | |
| PCSK9 153-162-KKK- | 99 | SIPWNLERIT-KKK-ϵK- |
| ϵK-EBV EBNA-1 Th | | PGPLRESIVCYFMVFLQTHI |
| PCSK9 153-162-KKK- | 100 | SIPWNLERIT-KKK-ϵK- |
| ϵK-C. tetani TT2 | | FNNFTVSFWLRVPKVSASHLE |
| Th | | |
| PCSK9 153-162-KKK- | 101 | SIPWNLERIT-KKK-ϵK- |
| ϵK-C. tetani TT3 | | KFIIKRYTPNNEIDSF |
| Th | | |
| PCSK9 153-162-KKK- | 102 | SIPWNLERIT-KKK-ϵK- |
| ϵK-C. tetani TT4 | | VSIDKFRIFCKALNPK |
| Th | | |
| PCSK9 153-162-KKK- | 103 | SIPWNLERIT-KKK-ϵK- |
| ϵK-EBV CP Th | | VPGLYSPCRAFFNKEELL |
| PCSK9 153-162-KKK- | 104 | SIPWNLERIT-KKK-ϵK- |
| ϵK-HCMV IE1 Th | | DKREMWMACIKELH |
| PCSK9 153-162-KKK- | 105 | SIPWNLERIT-KKK-ϵK- |
| ϵK-EBV GP340 Th | | TGHGARTSTEPTTDY |
| PCSK9 153-162-KKK- | 106 | SIPWNLERIT-KKK-ϵK- |
| ϵK-EBV BPLF1 Th | | KELKRQYEKKLRQ |
| PCSK9 153-162-KKK- | 107 | SIPWNLERIT-KKK-ϵK- |
| ϵK-EBV EBNA-2 Th | | TVFYNIPPMPL |

* Peptides are cyclized by cysteine disulfide bonds, as shown with the cysteines underlined.
The Cysteines/Serines that substitute the amino acids of the PCSK9 fragments are in italics.

TABLE 4
Immunogenicity Assessment in Guinea Pigs of PCSK9 Peptide Immunogen Constructs

| | | | | PCSK9₁₅₃₋₁₆₂ | | | | | PCSK9₃₆₈₋₃₈₂_mod | | | | |
|---|---|---|---|---|---|---|---|---|---|---|---|---|---|
| | | SEQ | | (SEQ ID NO: 2) | | | | | (SEQ ID NO: 6) | | | | |
| | Peptide immunogen | ID | Animal | ELISA Log₁₀ Titer | | | | | ELISA Log₁₀ Titer | | | | |
| Group # | description | NO: | No | 0 w | 3w | 6 w | 9 w | 12 w | 0 w | 3w | 6 w | 9 w | 12 w |
| 1 | Adjuvant control | | 7754 | 0.085 | 0.000 | 0.000 | 0.000 | 0.000 | 0.094 | 0.000 | 0.000 | 0.600 | 0.006 |
| | | | 7755 | 0.082 | 0.000 | 0.000 | 0.000 | 0.000 | 0.088 | 0.009 | 0.000 | 8.000 | 0.060 |
| | | | 7758 | 0.077 | 0.000 | 0.000 | 0.000 | 0.000 | 0.097 | 0.000 | 0.000 | 0.000 | 0.000 |
| 2 | PCSK9 153-162-KKK-εK- | 65 | 7757 | 0.072 | 5.143 | 6.678 | 5.629 | 5.287 | 0.082 | 0.000 | 0.000 | 0.000 | 0.000 |
| | UBITh1 | | 7758 | 0.069 | 5.280 | 5.790 | 5.194 | 5.024 | 0.111 | 0.000 | 0.000 | 0.000 | 0.000 |
| | | | 7759 | 0.066 | 4.978 | 5.638 | 5.268 | 5.068 | 0.084 | 0.000 | 0.000 | 0.000 | 0.000 |
| 3 | UBITh1-EK-KKK- | 68 | 7760 | 0.068 | 0.000 | 2.331 | 2.612 | 3.046 | 0.091 | 3.885 | 6.832 | 10.15 | 8.080 |
| | PCSK9 368-382_mod | | 7761 | 0.068 | 0.000 | 1.307 | 1.967 | 3.038 | 0.086 | 3.951 | 5.433 | 5.559 | 5.256 |
| | | | 7762 | 0.076 | 0.000 | 0.000 | 0.000 | 0.000 | 0.096 | 4.872 | 5.669 | 5.399 | 5.197 |
| 4 | PCSK9 368-382_mod- | 69 | 7763 | 0.079 | 1.600 | 4.867 | 5.141 | 5.182 | 0.087 | 0.000 | 4.415 | 4.527 | 4.274 |
| | KKK-εK-UBITh1 | | 7764 | 0.068 | 1.200 | 3.187 | 3.097 | 3.243 | 0.082 | 0.000 | 4.820 | 5.030 | 4.901 |
| | | | 7765 | 0.081 | 1.480 | 2.216 | 2.475 | 2.922 | 0.094 | 2.987 | 4.990 | 5.215 | 5.098 |
| 5 | UBITh1-εK-PCSK9 368- | 70 | 7766 | 0.074 | 0.000 | 2.984 | 3.369 | 4.582 | 0.078 | 5.117 | >10 | >10 | >10 |
| | 362 mod | | 7767 | 0.071 | 0.000 | 0.000 | 0.000 | 0.000 | 0.083 | 3.569 | 9.170 | 7.205 | 8.406 |
| | | | 7768 | 0.074 | 0.000 | 0.000 | 0.000 | 0.000 | 0.093 | 4.571 | 9.331 | 7.154 | 7.153 |
| 6 | PCSK9 388-382 mod-εK- | 71 | 7769 | 0.081 | 0.000 | 2.547 | 3.330 | 3.218 | 0.093 | 3.186 | 5.060 | 5.190 | 5.334 |
| | UBITh1 | | 7770 | 0.084 | 0.000 | 0.000 | 0.000 | 1.027 | 0.083 | 3.794 | 4.978 | 5.071 | 5.121 |
| | | | 7771 | 0.081 | 0.000 | 0.410 | 2.306 | 2.835 | 0.083 | 3.124 | 4.878 | 4.996 | 5.146 |
| 7 | UBITh1-εKKKK- | 72 | 7772 | 0.075 | 0.000 | 0.000 | 0.000 | 0.995 | 0.092 | 0.000 | 3.331 | 4.920 | 4.976 |
| | PCSK9 368-382 | | 7773 | 0.075 | 0.000 | 0.000 | 0.000 | 0.000 | 0.100 | 0.000 | 0.000 | 0.000 | 3.780 |
| | | | 7774 | 0.066 | 0.000 | 0.000 | 0.000 | 0.000 | 0.085 | 0.000 | 0.000 | 0.000 | 1.420 |
| 8 | PCSK 368-382-KKK-εK- | 73 | 7775 | 0.064 | 0.000 | 3.438 | 0.000 | 3.507 | 0.883 | 0.000 | 0.000 | 0.000 | 0.000 |
| | UBITh1 | | 7776 | 0.065 | 0.000 | 0.000 | 0.000 | 0.877 | 0.132 | 0.000 | 0.000 | 0.000 | 0.000 |
| | | | 7777 | 0.062 | 0.000 | 1.610 | 2.068 | 2.609 | 0.084 | 0.000 | 3.554 | 4.414 | 4.899 |
| 9 | PCSK9 368-882-EK- | 74 | 7778 | 0.086 | 0.000 | 0.000 | 0.000 | 0.409 | 0.079 | 0.000 | 1.079 | 1.374 | 1.253 |
| | UBITh1 | | 7779 | 0.071 | 0.000 | 0.000 | 0.000 | 0.000 | 0.072 | 0.000 | 0.000 | 0.000 | 0.000 |
| | | | 7780 | 0.066 | 0.000 | 0.000 | 0.000 | 0.000 | 0.065 | 0.000 | 0.000 | 0.000 | 0.000 |
| 10 | UBTh1-εK-KKK- | 75 | 7781 | 0.071 | 1.688 | 2.578 | 2.937 | 3.376 | 0.061 | 0.000 | 0.000 | 0.000 | 0.000 |
| | PCSK9 211-223 | | 7782 | 0.066 | 3.007 | 4.593 | 4.568 | 4.647 | 0.112 | 0.000 | 0.000 | 0.000 | 0.000 |
| | | | 7783 | 0.061 | 0.000 | 2.071 | 2.396 | 3.271 | 0.084 | 0.000 | 0.000 | 0.000 | 0.000 |
| 11 | PCSK9 211-223-KKK- | 76 | 7784 | 0.066 | 0.624 | 4.612 | 4.645 | 4.482 | 0.065 | 0.000 | 0.000 | 0.000 | 0.000 |
| | εK-UBITh1 | | 7785 | 0.075 | 1.240 | 2.692 | 3.312 | 3.275 | 0.087 | 0.000 | 0.000 | 0.000 | 0.000 |
| | | | 7788 | 0.073 | 0.000 | 0.000 | 1.418 | 1.739 | 0.090 | 0.000 | 0.000 | 0.000 | 0.000 |
| | | | | PCSK9₃₆₈₋₃₈₂ | | | | | PCSK9₂₁₁₋₂₂₃ | | | | |
| | | SEQ | | (SEQ ID NO: 5) | | | | | (SEQ ID NQ: 7) | | | | |
| | Peptide immunogen | ID | Animal | ELISA Log₁₀ Titer | | | | | ELISA Log₁₀ Titer | | | | |
| Group # | description | NO: | No | 0 w | 3 w | 6 w | 9 w | 12 w | 0 w | 3 w | 6 w | 9 w | 12 w |
| 1 | Adjuvant control | | 7754 | 0.094 | 0.000 | 0.000 | 0.000 | 0.000 | 0.086 | 0.000 | 0.000 | 0.000 | 0.000 |
| | | | 7755 | 0.074 | 0.000 | 0.000 | 0.000 | 0.000 | 0.088 | 0.000 | 0.000 | 0.000 | 0.000 |
| | | | 7758 | 0.069 | 0.000 | 0.000 | 0.000 | 0.000 | 0.094 | 0.000 | 0.000 | 0.000 | 0.000 |
| 2 | PCSK9 153-162-KKK-εK- | 65 | 7757 | 0.068 | 0.000 | 0.000 | 0.000 | 0.000 | 0.104 | 0.000 | 0.000 | 0.000 | 0.000 |
| | UBITh1 | | 7758 | 0.071 | 0.000 | 0.000 | 0.000 | 0.000 | 0.100 | 0.000 | 0.000 | 0.000 | 0.000 |
| | | | 7759 | 0.106 | 0.000 | 0.000 | 0.000 | 0.000 | 0.106 | 0.000 | 0.000 | 0.000 | 0.000 |
| 3 | UBITh1-EK-KKK- | 68 | 7760 | 0.078 | 0.000 | 3.369 | 3.218 | 3.272 | 0.106 | 0.000 | 0.000 | 0.000 | 1.331 |
| | PCSK9 368-382_mod | | 7761 | 0.093 | 0.000 | 3.400 | 3.770 | 3.505 | 0.088 | 0.000 | 5.031 | 5.079 | 4.995 |
| | | | 7762 | 0.105 | 0.000 | 4.847 | 4.918 | 4.947 | 0.077 | 0.000 | 2.338 | 2.553 | 2.382 |
| 4 | PCSK9 368-382_mod- | 69 | 7763 | 0.065 | 0.000 | 3.044 | 0.163 | 0.000 | 0.087 | 0.000 | 0.000 | 0.000 | 0.000 |
| | KKK-εK-UBITh1 | | 7764 | 0.056 | 0.000 | 0.284 | 0.000 | 0.000 | 0.078 | 0.000 | 0.000 | 0.000 | 0.000 |
| | | | 7765 | 0.061 | 0.000 | 0.000 | 0.000 | 0.044 | 0.084 | 0.000 | 0.000 | 0.000 | 0.000 |
| 5 | UBITh1-εK-PCSK9 368- | 70 | 7766 | 0.056 | 4.888 | 8.887 | >10 | >10 | 0.076 | 3.769 | 4.923 | 4.707 | 4.618 |
| | 362 mod | | 7767 | 0.057 | 2.480 | 5.083 | 5.066 | 5.227 | 0.075 | 2.489 | 4.999 | 5.252 | 5.506 |
| | | | 7768 | 0.058 | 3.390 | 5.442 | 5.464 | 5.898 | 0.100 | 2.898 | 4.584 | 4.697 | 4.786 |
| 8 | PCSK9 388-382 mod-εK- | 71 | 7769 | 0.063 | 0.000 | 0.000 | 0.000 | 0.000 | 0.110 | 0.000 | 0.000 | 0.000 | 0.000 |
| | UBITh1 | | 7770 | 0.088 | 0.000 | 0.000 | 0.000 | 0.000 | 0.078 | 0.000 | 0.000 | 0.000 | 0.000 |
| | | | 7771 | 0.070 | 0.000 | 0.000 | 0.000 | 0.000 | 0.067 | 0.000 | 0.000 | 0.000 | 0.000 |
| 7 | UBITh1-εKKKK- | 72 | 7772 | 0.065 | 4.653 | 5.249 | 5.380 | 5.561 | 0.064 | 0.000 | 0.000 | 0.000 | 0.000 |
| | PCSK9 368-382 | | 7773 | 0.069 | 4.764 | 9.827 | 6.037 | 5.919 | 0.068 | 0.000 | 0.000 | 0.000 | 2.213 |
| | | | 7774 | 0.063 | 4.605 | 5.785 | 5.507 | 5.697 | 0.063 | 0.000 | 0.000 | 0.000 | 0.000 |
| 8 | PCSK 368-382-KKK-εK- | 73 | 7775 | 0.069 | 3.548 | 4.582 | 3.438 | 3.990 | 0.071 | 0.000 | 0.000 | 0.000 | 0.000 |
| | UBITh1 | | 7776 | 0.073 | 3.174 | 4.136 | 3.259 | 3.590 | 0.085 | 0.000 | 0.000 | 0.000 | 0.000 |
| | | | 7777 | 0.072 | 3.182 | 5.040 | 5.038 | 4.934 | 0.074 | 0.000 | 0.000 | 0.000 | 1.731 |
| 9 | PCSK9 368-882-EK- | 74 | 7778 | 0.085 | 3.182 | 4.847 | 4.929 | 4.944 | 0.074 | 0.000 | 0.000 | 0.000 | 0.000 |
| | UBITh1 | | 7779 | 0.070 | 3.188 | 3.367 | 3.168 | 4.703 | 0.092 | 0.000 | 0.000 | 0.000 | 0.000 |
| | | | 7780 | 0.071 | 3.443 | 4.846 | 4.746 | 4.609 | 0.081 | 0.000 | 0.000 | 0.000 | 0.000 |
| 10 | UBTh1-εK-KKK- | 75 | 7781 | 0.068 | 0.000 | 0.000 | 0.000 | 0.000 | 0.065 | 4.929 | 5.206 | 5.066 | 5.013 |
| | PCSK9 211-223 | | 7782 | 0.074 | 0.000 | 0.000 | 0.000 | 0.000 | 0.079 | 5.226 | 6.681 | 6.132 | 5.677 |
| | | | 7783 | 0.080 | 0.000 | 0.000 | 0.000 | 0.000 | 0.059 | 5.003 | 5.149 | 5.125 | 5.130 |
| 11 | PCSK9 211-223-KKK- | 76 | 7784 | 0.097 | 0.000 | 0.000 | 0.000 | 0.000 | 0.068 | 5.875 | >10 | 9.416 | 7.453 |
| | εK-UBITh1 | | 7785 | 0.120 | 0.000 | 0.000 | 0.000 | 0.000 | 0.074 | 5.063 | >10 | 9.460 | 7.858 |
| | | | 7788 | 0.111 | 0.000 | 0.000 | 0.000 | 0.000 | 0.077 | 4.724 | 5.172 | 5.334 | 5.309 |

TABLE 5
Ranking of the peptide immunogens based on functional assays

| Assay for | Ranking | Supporting FIG. Nos. |
|---|---|---|
| Binging rPCSK9 protein | SEQ ID NO: | 7 |
| (ELISA titer for matured | 65 > 76 > 75 > 74 > 73 > 72 > 68 = 69 = 70 = 71 | |
| form of PCSK9) | | |
| T-CHO inhibition rate (%) | SEQ ID NO: | 9A-9B |
| | 65 > 75 > 70 > 71 > 74 > 68 > 73 > 69 > 72 > 76 | |
| LDL inhibition rate (%) | SEQ ID NO: | 8A-8B |
| | 65 > 75 > 70 > 71 > 73 > 74 > 69 > 76 > 68 > 72 | |
| LDL uptake ratio (%) in vitro | SEQ ID NO: | 10A-10C |
| | 75 > 65 > 76 > 68 > 72 > 69 = 70 = 73 > 71 = 74 | |

TABLE 6
Immunogenicity Study

| | | | | Immunogen | |
|---|---|---|---|---|---|
| | | | | dose/ | |
| | | Ani- | | volume/ | |
| | | mals/ | Formu- | immuniza- | Shot |
| Group | Description | Group | lation | tion site | No. |
| 1 | PCSK9₁₅₃₋₁₆₅- | 3 | ISA | Prime; | 5 |
| | KKK-ϵK- | | 51VG | 400 μg/ | (0, 3, |
| | | | CpG3 | 1.0 mL/ | 6, 9, |
| | | | 100 | dose/I.M. | 12 wpi) |
| | UBITh1 | | μg/mL | (four | |
| | (SEQ ID | | 0.2% | injection | |
| | NO: 66) | | TWEEN80 | sites, | |
| | | | | 0.25 mL/ | |
| | | | | site) | |
| | | | | Boosts: | |
| | | | | 100 μg/ | |
| | | | | 0.25 m:/ | |
| | | | | dose/im | |
| 2 | PCSK9₁₅₃₋₁₇₂- | | | | |
| | KKK-ϵK- | | | | |
| | UBITh1 | | | | |
| | (SEQ ID | | | | |
| | NO: 67) | | | | |

1. A PCSK9 peptide immunogen construct having about 20 or more amino acids, represented by the formulae:
(Th)ₘ-(A)ₙ-(PCSK9 functional B cell epitope peptide)-X
or
(PCSK9 functional B cell epitope peptide)-(A)ₙ-(Th)ₘ-X
or
(Th)ₘ-(A)ₙ-(PCSK9 functional B cell epitope peptide)-(A)ₙ-(Th)ₘ-X
wherein
Th is a heterologous T helper epitope;
A is a heterologous spacer;
(PCSK9 functional B cell epitope peptide) is a B cell epitope peptide having from 7 to about 30 amino acid residues derived from the catalytic domain of the PCSK9 protein (SEQ ID NO: 111);
X is an α-COOH or α-CONH₂ of an amino acid;
m is from 1 to about 4; and
n is from 0 to about 10.

2. The PCSK9 peptide immunogen construct according to claim 1, wherein the PCSK9 functional B cell epitope peptide is selected from the group consisting of SEQ ID NOs: 2-9.

3. The PCSK9 peptide immunogen construct according to claim 1, wherein the Th epitope is selected from the group consisting of SEQ ID NOs: 13-64.

4. The PCSK9 peptide immunogen construct according to claim 1, wherein the PCSK9 functional B cell epitope peptide is selected from the group consisting of SEQ ID NOs: 2-9 and the Th epitope is selected from the group consisting of SEQ ID NOs: 13-64.

5. The PCSK9 peptide immunogen construct according to claim 1, wherein the peptide immunogen construct is selected from the group consisting of SEQ ID NOs: 65-107.

6. A PCSK9 peptide immunogen construct comprising:
a. a B cell epitope comprising from about 7 to about 30 amino acid residues from the catalytic domain of the PCSK9 sequence of SEQ ID NO: 111;
b. a T helper epitope comprising an amino acid sequence selected from the group consisting of SEQ ID NOs: 13-64, and any combination thereof; and
c. an optional heterologous spacer selected from the group consisting of an amino acid, Lys-, Gly-, Lys-Lys-Lys-, (α, ε-N)Lys, ε-N-Lys-Lys-Lys-Lys (SEQ ID NO: 11), Lys-Lys-Lys-ε-N-Lys (SEQ ID NO: 12), and Pro-Pro-Xaa-Pro-Xaa-Pro (SEQ ID NO: 10), and any combination thereof,
wherein the B cell epitope is covalently linked to the T helper epitope directly or through the optional heterologous spacer.

7. The PCSK9 peptide immunogen construct of claim 6, wherein the B cell epitope is selected from the group consisting of SEQ ID NOs: 2-9.

8. The PCSK9 peptide immunogen construct of claim 6, wherein the optional heterologous spacer is (α, ε-N)Lys, ε-N-Lys-Lys-Lys-Lys (SEQ ID NO: 11), Lys-Lys-Lys-ε-N-Lys (SEQ ID NO: 12), or Pro-Pro-Xaa-Pro-Xaa-Pro (SEQ ID NO: 10), where Xaa is any amino acid.

9. The PCSK9 peptide immunogen construct of claim 6, wherein the T helper epitope is covalently linked to the amino- or carboxyl-terminus of the B cell epitope.

10. The PCSK9 peptide immunogen construct of claim 6, wherein the T helper epitope is covalently linked to the amino- or carboxyl-terminus of the B cell epitope through the optional heterologous spacer.

11. A composition comprising the PCSK9 peptide immunogen construct according to claim 1.

12. A pharmaceutical composition comprising:
a. the PCSK9 peptide immunogen construct according to claim 1; and
b. a pharmaceutically acceptable delivery vehicle and/or adjuvant.

13. The pharmaceutical composition of claim 12, wherein
a. the PCSK9 functional B cell epitope peptide is selected from the group consisting of SEQ ID NOs: 2-9;
b. the Th epitope is selected from the group consisting of SEQ ID NOs: 13-64; and
c. the heterologous spacer is selected from the group consisting of an amino acid, Lys-, Gly-, Lys-Lys-Lys-, (α, ε-N)Lys, ε-N-Lys-Lys-Lys-Lys (SEQ ID NO: 11), Lys-Lys-Lys-ε-N-Lys (SEQ ID NO: 12), and Pro-Pro-Xaa-Pro-Xaa-Pro (SEQ ID NO: 10), and any combination thereof; and
wherein the PCSK9 peptide immunogen construct is mixed with an CpG oligodeoxynucleotide (ODN) to form a stabilized immunostimulatory complex.

14. The pharmaceutical composition of claim 12, wherein
a. the PCSK9 peptide immunogen construct is selected from the group consisting of SEQ ID NOs: 65-107; and
wherein the PCSK9 peptide immunogen construct is mixed with an CpG oligodeoxynucleotide (ODN) to form a stabilized immunostimulatory complex.

15. A method for generating antibodies against PCSK9 in an animal comprising administering the pharmaceutical composition according to claim 12 to the animal.

16. An isolated antibody or epitope-binding fragment thereof that specifically binds to the PCSK9 and LDL-R receptor binding region of SEQ ID NOs: 2-9.

17. The isolated antibody or epitope-binding fragment thereof according to claim 16 bound to the PCSK9 peptide immunogen construct.

18. A composition comprising the isolated antibody or epitope-binding fragment thereof according to claim 16.

19. A method of preventing and/or treating patients with PCSK9 mediated disorders including an increased serum level of low-density lipoprotein cholesterol (LDL-C) and CV events in an animal comprising administering the pharmaceutical composition of claim 12 to the animal.


SEQUENCE LISTING (115 sequences)

SEQ ID NO: 1 - protein, 692 aa, Homo sapiens
  PEPTIDE (1)..(692): Human PCSK9
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 2 - protein, 10 aa, Homo sapiens
  PEPTIDE (1)..(10): PCSK9 (153-162)
SIPWNLERIT

SEQ ID NO: 3 - protein, 13 aa, Homo sapiens
  PEPTIDE (1)..(13): PCSK9 (153-165)
SIPWNLERITPPR

SEQ ID NO: 4 - protein, 20 aa, Homo sapiens
  PEPTIDE (1)..(20): PCSK9 (153-172)
SIPWNLERITPPRYRADEYQ

SEQ ID NO: 5 - protein, 15 aa, Homo sapiens
  PEPTIDE (1)..(15): PCSK9 (368-382)
IIGASSDCSTCFVSQ

SEQ ID NO: 6 - protein, 15 aa, Artificial Sequence
  synthetic peptide
  PEPTIDE (1)..(15): PCSK9 (368-382_mod) (I368 to C368, C375 to S375, C378 to S378, Q382 to C382)
CIGASSDSSTSFVSC

SEQ ID NO: 7 - protein, 13 aa, Homo sapiens
  PEPTIDE (1)..(13): PCSK9 (211-223)
CDGTRFHRQASKC

SEQ ID NO: 8 - protein, 10 aa, Cavia porcellus
  PEPTIDE (1)..(10): Guinea Pig PCSK9 (153-162)
SVPWNLERIL

SEQ ID NO: 9 - protein, 15 aa, Cavia porcellus
  PEPTIDE (1)..(15): Monkey / Guinea Pig PCSK9 (368-382)
IIGASSDCSTCFVSR

SEQ ID NO: 10 - protein, 6 aa, Artificial Sequence
  Synthetic peptide
  PEPTIDE (1)..(6): Flexible Hinge Spacer
  SITE (3)..(3): Xaa is any amino acid, preferably Asp
  SITE (5)..(5): Xaa is any amino acid, preferably Asp
PPXPXP

SEQ ID NO: 11 - protein, 4 aa, Artificial Sequence
  Synthetic peptide
  SITE (1)..(1): epsilon-K
  PEPTIDE (1)..(4): epsilon-K-KKK as a spacer
KKKK

SEQ ID NO: 12 - protein, 4 aa, Artificial Sequence
  Synthetic peptide
  PEPTIDE (1)..(4): KKK-epsilon-K as a spacer
  SITE (4)..(4): epsilon-K
KKKK

SEQ ID NO: 13 - protein, 17 aa, Clostridium tetani
  PEPTIDE (1)..(17): Clostridium tetani 1 Th
KKQYIKANSKFIGITEL

SEQ ID NO: 14 - protein, 15 aa, Measles virus
  PEPTIDE (1)..(15): MvF1 Th
LSEIKGVIVHRLEGV

SEQ ID NO: 15 - protein, 24 aa, Bordetella pertussis
  PEPTIDE (1)..(24): Bordetella pertussis Th
GAYARCPNGTRALTVAELRGNAEL

SEQ ID NO: 16 - protein, 17 aa, Clostridium tetani
  PEPTIDE (1)..(17): Clostridium tetani 2 Th
WVRDIIDDFTNESSQKT

SEQ ID NO: 17 - protein, 23 aa, diphtheria bacilli
  PEPTIDE (1)..(23): Diphtheria Th
DSETADNLEKTVAALSILPGHGC

SEQ ID NO: 18 - protein, 21 aa, Plasmodium falciparum
  PEPTIDE (1)..(21): Plasmodium falciparum Th
DHEKKHAKMEKASSVFNVVNS

SEQ ID NO: 19 - protein, 17 aa, Schistosoma mansoni
  PEPTIDE (1)..(17): Schistosoma mansoni Th
KWFKTNAPNGVDEKHRH

SEQ ID NO: 20 - protein, 25 aa, Cholera Toxin
  PEPTIDE (1)..(25): Cholera Toxin Th
ALNIWDRFDVFCTLGATTGYLKGNS

SEQ ID NO: 21 - protein, 15 aa, Measles virus
  PEPTIDE (1)..(15): MvF 2 Th
ISEIKGVIVHKIEGI

SEQ ID NO: 22 - protein, 22 aa, Measles virus
  PEPTIDE (1)..(22): KKKMvF3 Th
KKKISISEIKGVIVHKIEGILF

SEQ ID NO: 23 - protein, 22 aa, Measles virus
  PEPTIDE (1)..(22): KKKMvF3 Th
KKKISITEIRTVIVTRIETILF

SEQ ID NO: 24 - protein, 22 aa, Measles virus
  PEPTIDE (1)..(22): KKKMvF 3 Th
  SITE (7)..(7): S or T
  SITE (10)..(10): K or R
  SITE (11)..(11): G or T
  SITE (15)..(15): H or T
  SITE (16)..(16): K or R
  SITE (19)..(19): G or T
KKKISIXEIXXVIVXXIEXILF

SEQ ID NO: 25 - protein, 18 aa, Hepatitis B virus
  PEPTIDE (1)..(18): HBsAg1 Th
KKKLFLLTKLLTLPQSLD

SEQ ID NO: 26 - protein, 18 aa, Hepatitis B virus
  PEPTIDE (1)..(18): HBsAg1 Th
RRRIKIITRIITIPLSIR

SEQ ID NO: 27 - protein, 18 aa, Hepatitis B virus
  PEPTIDE (1)..(18): HBsAg1 Th
KKKVRVVTKVVTVPISVD

SEQ ID NO: 28 - protein, 18 aa, Hepatitis B virus
  PEPTIDE (1)..(18): HBsAg1 Th
KKKFFFFTKFFTFPVSFD

SEQ ID NO: 29 - protein, 18 aa, Hepatitis B virus
  PEPTIDE (1)..(18): HBsAg1 Th
KKKLFLLTKLLTLPFSLD

SEQ ID NO: 30 - protein, 18 aa, Hepatitis B virus
  PEPTIDE (1)..(18): HBsAg 1 Th
  SITE (1)..(1): K or R
  SITE (2)..(2): K or R
  SITE (3)..(3): K or R
  SITE (4)..(4): L or I or V or F
  SITE (5)..(5): F or K or R
  SITE (6)..(6): L or I or V or F
  SITE (7)..(7): L or I or V or F
  SITE (9)..(9): K or R
  SITE (10)..(10): L or I or V or F
  SITE (11)..(11): L or I or V or F
  SITE (13)..(13): L or I or V or F
  SITE (15)..(15): Q or L or I or V or F
  SITE (17)..(17): L or I or V or F
  SITE (18)..(18): D or R
XXXXXXXTXXXTXPXSXX

SEQ ID NO: 31 - protein, 19 aa, Measles virus
  PEPTIDE (1)..(19): MvF4 Th
ISISEIKGVIVHKIETILF

SEQ ID NO: 32 - protein, 19 aa, Measles virus
  PEPTIDE (1)..(19): MvF4 Th
ISITEIRTVIVTRIETILF

SEQ ID NO: 33 - protein, 19 aa, Measles virus
  PEPTIDE (1)..(19): MvF4 Th  (UBITh3)
  SITE (4)..(4): S or T
  SITE (7)..(7): K or R
  SITE (8)..(8): G or T
  SITE (12)..(12): H or T
  SITE (13)..(13): K or R
ISIXEIXXVIVXXIETILF

SEQ ID NO: 34 - protein, 18 aa, Hepatitis B virus
  PEPTIDE (1)..(18): HBsAg2 Th
KKKIITITRIITIPQSLD

SEQ ID NO: 35 - protein, 18 aa, Hepatitis B virus
  PEPTIDE (1)..(18): HBsAg2 Th
KKKFFLLTRILTIITTID

SEQ ID NO: 36 - protein, 18 aa, Hepatitis B virus
  PEPTIDE (1)..(18): HBsAg 2 Th
  SITE (4)..(4): I or F
  SITE (5)..(5): I or F
  SITE (6)..(6): T or L
  SITE (7)..(7): I or L
  SITE (11)..(11): I or L
  SITE (14)..(14): P or I
  SITE (15)..(15): Q or T
  SITE (16)..(16): S or T
  SITE (17)..(17): L or I
KKKXXXXTRIXTIXXXXD

SEQ ID NO: 37 - protein, 19 aa, Measles virus
  PEPTIDE (1)..(19): MvF5 Th (UBITh1)
ISITEIKGVIVHRIETILF

SEQ ID NO: 38 - protein, 18 aa, Hepatitis B virus
  PEPTIDE (1)..(18): HBsAg 3 Th
KKKIITITRIITIITTID

SEQ ID NO: 39 - protein, 11 aa, Influenza virus
  PEPTIDE (1)..(11): Influenza Matrix protein 1_1 Th
FVFTLTVPSER

SEQ ID NO: 40 - protein, 15 aa, Influenza virus
  PEPTIDE (1)..(15): Influenza Matrix protein 1_2 Th
SGPLKAEIAQRLEDV

SEQ ID NO: 41 - protein, 9 aa, Influenza virus
  PEPTIDE (1)..(9): Influenza Non-structural protein 1 Th
DRLRRDQKS

SEQ ID NO: 42 - protein, 19 aa, Epstein-Barr virus
  PEPTIDE (1)..(19): EBV BHRF1 Th
AGLTLSLLVICSYLFISRG

SEQ ID NO: 43 - protein, 15 aa, Clostridium tetani
  PEPTIDE (1)..(15): Clostridium tetani TT1 Th
QYIKANSKFIGITEL

SEQ ID NO: 44 - protein, 20 aa, Epstein-Barr virus
  PEPTIDE (1)..(20): EBV EBNA-1 Th
PGPLRESIVCYFMVFLQTHI

SEQ ID NO: 45 - protein, 21 aa, Clostridium tetani
  PEPTIDE (1)..(21): Clostridium tetani  TT2 Th
FNNFTVSFWLRVPKVSASHLE

SEQ ID NO: 46 - protein, 16 aa, Clostridium tetani
  PEPTIDE (1)..(16): Clostridium tetani TT3 Th
KFIIKRYTPNNEIDSF

SEQ ID NO: 47 - protein, 16 aa, Clostridium tetani
  PEPTIDE (1)..(16): Clostridium tetani  TT4 Th
VSIDKFRIFCKALNPK

SEQ ID NO: 48 - protein, 18 aa, Epstein-Barr virus
  PEPTIDE (1)..(18): EBV CP Th
VPGLYSPCRAFFNKEELL

SEQ ID NO: 49 - protein, 14 aa, Human cytomegalovirus
  PEPTIDE (1)..(14): HCMV IE1 Th
DKREMWMACIKELH

SEQ ID NO: 50 - protein, 15 aa, Epstein-Barr virus
  PEPTIDE (1)..(15): EBV GP340 Th
TGHGARTSTEPTTDY

SEQ ID NO: 51 - protein, 13 aa, Epstein-Barr virus
  PEPTIDE (1)..(13): EBV BPLF1 Th
KELKRQYEKKLRQ

SEQ ID NO: 52 - protein, 11 aa, Epstein-Barr virus
  PEPTIDE (1)..(11): EBV EBNA-2 Th
TVFYNIPPMPL

SEQ ID NO: 53 - protein, 16 aa, Measles virus
  PEPTIDE (1)..(16): MvF Th
DLSDLKGLLLHKLDGL

SEQ ID NO: 54 - protein, 16 aa, Measles virus
  PEPTIDE (1)..(16): MvF Th
EISDEIRLIIIKRIEI

SEQ ID NO: 55 - protein, 16 aa, Measles virus
  PEPTIDE (1)..(16): MvF Th
DVSDVKGVVVHKVDGV

SEQ ID NO: 56 - protein, 16 aa, Measles virus
  PEPTIDE (1)..(16): MvF Th
DFSDFKGFFFHKFDGF

SEQ ID NO: 57 - protein, 16 aa, Measles virus
  PEPTIDE (1)..(16): MvF Th
  SITE (1)..(1): D or E
  SITE (2)..(2): L or I or V or F
  SITE (4)..(4): D or E
  SITE (5)..(5): L or I or V or F
  SITE (6)..(6): K or R
  SITE (8)..(8): L or I or V or F
  SITE (9)..(9): L or I or V or F
  SITE (10)..(10): L or I or V or F
  SITE (12)..(12): K or R
  SITE (13)..(13): L or I or V or F
  SITE (14)..(14): D or E
  SITE (16)..(16): L or I or V or F
XXSXXXGXXXHXXXGX

SEQ ID NO: 58 - protein, 19 aa, Measles virus
  PEPTIDE (1)..(19): MvF3 Th
ISISEIKGVIVHKIEGILF

SEQ ID NO: 59 - protein, 19 aa, Measles virus
  PEPTIDE (1)..(19): MvF 3Th
ISITEIRTVIVTRIETILF

SEQ ID NO: 60 - protein, 19 aa, Measles virus
  PEPTIDE (1)..(19): MvF3 Th
  SITE (4)..(4): S or T
  SITE (7)..(7): K or R
  SITE (8)..(8): G or T
  SITE (12)..(12): H or T
  SITE (13)..(13): K or R
  SITE (16)..(16): G or T
ISIXEIXXVIVXXIEXILF

SEQ ID NO: 61 - protein, 22 aa, Measles virus
  PEPTIDE (1)..(22): KKKMvF5 Th (UBITh1a)
KKKISITEIKGVIVHRIETILF

SEQ ID NO: 62 - protein, 15 aa, Hepatitis B virus
  PEPTIDE (1)..(15): HBsAg4 Th (UBITh4)
FFLLTRILTIPQSLD

SEQ ID NO: 63 - protein, 18 aa, Hepatitis B virus
  PEPTIDE (1)..(18): KKK-HBsAg Th
KKKFFLLTRILTIPQSLD

SEQ ID NO: 64 - protein, 14 aa, Hepatitis B virus
  PEPTIDE (1)..(14): HBsAg Th
FFLLTRILTIPQSL

SEQ ID NO: 65 - protein, 33 aa, Artificial Sequence
  Synthetic peptide
  PEPTIDE (1)..(10): PCSK9 153-162
  PEPTIDE (11)..(14): KKK-epsilon K as a spacer
  SITE (14)..(14): epsilon K
  PEPTIDE (15)..(33): MvF5 Th (UBITh1)
SIPWNLERITKKKKISITEIKGVIVHRIETILF

SEQ ID NO: 66 - protein, 36 aa, Artificial Sequence
  Synthetic peptide
  PEPTIDE (1)..(13): PCSK9 153-165
  PEPTIDE (14)..(17): KKK-epsilon K as a spacer
  SITE (17)..(17): epsilon K
  PEPTIDE (18)..(36): MvF5 Th (UBITh1)
SIPWNLERITPPRKKKKISITEIKGVIVHRIETILF

SEQ ID NO: 67 - protein, 43 aa, Artificial Sequence
  Synthetic peptide
  PEPTIDE (1)..(20): PCSK9 153-172
  PEPTIDE (21)..(24): KKK-epsilon K as a spacer
  SITE (24)..(24): epsilon K
  PEPTIDE (25)..(43): MvF5 Th (UBITh1)
SIPWNLERITPPRYRADEYQKKKKISITEIKGVIVHRIETILF

SEQ ID NO: 68 - protein, 38 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(38): PCSK9 368-382_mod
ISITEIKGVIVHRIETILFKKKKCIGASSDSSTSFVSC

SEQ ID NO: 69 - protein, 38 aa, Artificial Sequence
  Synthetic peptide
  PEPTIDE (1)..(15): PCSK9 368-382_mod
  PEPTIDE (16)..(19): KKK-epsilon K as a spacer
  SITE (19)..(19): epsilon K
  PEPTIDE (20)..(38): MvF5 Th (UBITh1)
CIGASSDSSTSFVSCKKKKISITEIKGVIVHRIETILF

SEQ ID NO: 70 - protein, 35 aa, Artificial Sequence
  Synthetic peptide
  PEPTIDE (1)..(19): MvF5 Th (UBITh1)
  SITE (20)..(20): epsilon-K
  PEPTIDE (21)..(35): PCSK9 368-382_mod
ISITEIKGVIVHRIETILFKCIGASSDSSTSFVSC

SEQ ID NO: 71 - protein, 35 aa, Artificial Sequence
  Synthetic peptide
  PEPTIDE (1)..(15): PCSK9 368-382_mod
  SITE (16)..(16): epsilon K
  PEPTIDE (17)..(35): MvF5 Th (UBITh1)
CIGASSDSSTSFVSCKISITEIKGVIVHRIETILF

SEQ ID NO: 72 - protein, 38 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(38): PCSK9 368-382
ISITEIKGVIVHRIETILFKKKKIIGASSDCSTCFVSQ

SEQ ID NO: 73 - protein, 38 aa, Artificial Sequence
  Synthetic peptide
  PEPTIDE (1)..(15): PCSK9 368-382
  PEPTIDE (16)..(19): KKK-epsilon K as a spacer
  SITE (19)..(19): epsilon K
  PEPTIDE (20)..(38): MvF5 Th (UBITh1)
IIGASSDCSTCFVSQKKKKISITEIKGVIVHRIETILF

SEQ ID NO: 74 - protein, 35 aa, Artificial Sequence
  Synthetic peptide
  PEPTIDE (1)..(15): PCSK9 368-382
  SITE (16)..(16): epsilon K
  PEPTIDE (17)..(35): MvF5 Th (UBITh1)
IIGASSDCSTCFVSQKISITEIKGVIVHRIETILF

SEQ ID NO: 75 - protein, 36 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(36): PCSK9 211-223
ISITEIKGVIVHRIETILFKKKKCDGTRFHRQASKC

SEQ ID NO: 76 - protein, 36 aa, Artificial Sequence
  Synthetic peptide
  PEPTIDE (1)..(13): PCSK9 211-223
  PEPTIDE (14)..(17): KKK-epsilon K as a spacer
  SITE (17)..(17): epsilon K
  PEPTIDE (18)..(36): MvF5 Th (UBITh1)
CDGTRFHRQASKCKKKKISITEIKGVIVHRIETILF

SEQ ID NO: 77 - protein, 33 aa, Artificial Sequence
  Synthetic peptide
  PEPTIDE (1)..(10): Guinea Pig  PCSK9 153-162
  PEPTIDE (11)..(14): KKK-epsilon K as a spacer
  SITE (14)..(14): epsilon K
  PEPTIDE (15)..(33): MvF5 Th (UBITh1)
SVPWNLERILKKKKISITEIKGVIVHRIETILF

SEQ ID NO: 78 - protein, 38 aa, Artificial Sequence
  Synthetic peptide
  PEPTIDE (1)..(19): MvF5 Th (UBITh1)
  SITE (20)..(20): epsilon-K
  PEPTIDE (20)..(23): epsilon K-KKK as a spacer
  PEPTIDE (24)..(38): Guinea Pig  PCSK9 368-382
ISITEIKGVIVHRIETILFKKKKIIGASSDCSTCFVSR

SEQ ID NO: 79 - protein, 38 aa, Artificial Sequence
  Synthetic peptide
  PEPTIDE (1)..(15): Guinea Pig PCSK9 368-382
  PEPTIDE (16)..(19): KKK-epsilon K as a spacer
  SITE (19)..(19): epsilon K
  PEPTIDE (20)..(38): MvF5 Th (UBITh1)
IIGASSDCSTCFVSRKKKKISITEIKGVIVHRIETILF

SEQ ID NO: 80 - protein, 32 aa, Artificial Sequence
  Peptide immunogen construct
  PEPTIDE (1)..(10): PCSK9 153-162
  PEPTIDE (1)..(10): PCSK9 (153-162)
  PEPTIDE (11)..(14): epsilon-K-KKK spacer
  SITE (14)..(14): epsilon-K
  PEPTIDE (15)..(32): HBsAg3 Th (UBITh2)
SIPWNLERITKKKKKKKIITITRIITIITTID

SEQ ID NO: 81 - protein, 33 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(33): MvF4 Th  (UBITh3)
  SITE (18)..(18): S or T
  SITE (21)..(21): K or R
  SITE (22)..(22): G or T
  SITE (26)..(26): H or T
  SITE (27)..(27): K or R
SIPWNLERITKKKKISIXEIXXVIVXXIETILF

SEQ ID NO: 82 - protein, 31 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(31): Clostridium tetani1 Th
SIPWNLERITKKKKKKQYIKANSKFIGITEL

SEQ ID NO: 83 - protein, 29 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(29): MvF1 Th
SIPWNLERITKKKKLSEIKGVIVHRLEGV

SEQ ID NO: 84 - protein, 38 aa, Artificial Sequence
  Synthetic peptide
  PEPTIDE (1)..(10): PCSK9 (153-162)
  PEPTIDE (11)..(14): epsilon-K-KKK spacer
  SITE (14)..(14): epsilon-K
  PEPTIDE (15)..(38): Bordetella pertussis Th
SIPWNLERITKKKKGAYARCPNGTRALTVAELRGNAEL

SEQ ID NO: 85 - protein, 31 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(31): Clostridium tetani2 Th
SIPWNLERITKKKKWVRDIIDDFTNESSQKT

SEQ ID NO: 86 - protein, 37 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(37): Diphtheria Th
SIPWNLERITKKKKDSETADNLEKTVAALSILPGHGC

SEQ ID NO: 87 - protein, 35 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(35): Plasmodium falciparum Th
SIPWNLERITKKKKDHEKKHAKMEKASSVFNVVNS

SEQ ID NO: 88 - protein, 31 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(31): Schistosoma mansoni Th
SIPWNLERITKKKKKWFKTNAPNGVDEKHRH

SEQ ID NO: 89 - protein, 39 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(39): Cholera Toxin Th
SIPWNLERITKKKKALNIWDRFDVFCTLGATTGYLKGNS

SEQ ID NO: 90 - protein, 29 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(29): MvF2 Th
SIPWNLERITKKKKISEIKGVIVHKIEGI

SEQ ID NO: 91 - protein, 36 aa, Artificial Sequence
  Synthetic peptide
  PEPTIDE (1)..(10): PCSK9 (153-162)
  PEPTIDE (11)..(14): epsilon-K-KKK spacer
  SITE (14)..(14): epsilon K as a spacer
  PEPTIDE (15)..(36): KKKMvF3 Th
  SITE (21)..(21): S or T
  SITE (24)..(24): K or R
  SITE (25)..(25): G or T
  SITE (29)..(29): H or T
  SITE (30)..(30): K or R
  SITE (33)..(33): G or T
SIPWNLERITKKKKKKKISIXEIXXVIVXXIEXILF

SEQ ID NO: 92 - protein, 32 aa, Artificial Sequence
  Synthetic peptide
  PEPTIDE (1)..(10): PCSK9 (153-162)
  PEPTIDE (11)..(14): epsilon-K-KKK spacer
  SITE (14)..(14): epsilon K as a spacer
  PEPTIDE (15)..(32): HBsAg 1 Th
  SITE (15)..(15): K or R
  SITE (16)..(16): K or R
  SITE (17)..(17): K or R
  SITE (18)..(18): L or I or V or F
  SITE (19)..(19): F or K or R
  SITE (20)..(20): L or I or V or F
  SITE (21)..(21): L or I or V or F
  SITE (23)..(23): K or R
  SITE (24)..(24): L or I or V or F
  SITE (25)..(25): L or I or V or F
  SITE (27)..(27): L or I or V or F
  SITE (29)..(29): Q or L or I or V or F
  SITE (31)..(31): L or I or V or F
  SITE (32)..(32): D or R
SIPWNLERITKKKKXXXXXXXTXXXTXPXSXX

SEQ ID NO: 93 - protein, 32 aa, Artificial Sequence
  Synthetic peptide
  PEPTIDE (1)..(10): PCSK9 (153-162)
  PEPTIDE (11)..(14): epsilon-K-KKK spacer
  SITE (14)..(14): epsilon K as a spacer
  PEPTIDE (15)..(32): HBsAg 2 Th
  SITE (18)..(18): I or F
  SITE (19)..(19): I or F
  SITE (20)..(20): T or L
  SITE (21)..(21): I or L
  SITE (25)..(25): I or L
  SITE (28)..(28): P or I
  SITE (29)..(29): Q or T
  SITE (30)..(30): S or T
  SITE (31)..(31): L or I
SIPWNLERITKKKKKKKXXXXTRIXTIXXXXD

SEQ ID NO: 94 - protein, 25 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(25): Influenza MP1_1 Th
SIPWNLERITKKKKFVFTLTVPSER

SEQ ID NO: 95 - protein, 29 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(29): Influenza MP1_2 Th
SIPWNLERITKKKKSGPLKAEIAQRLEDV

SEQ ID NO: 96 - protein, 23 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(23): Influenza NSP1 Th
SIPWNLERITKKKKDRLRRDQKS

SEQ ID NO: 97 - protein, 33 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(33): EBV BHRF1 Th
SIPWNLERITKKKKAGLTLSLLVICSYLFISRG

SEQ ID NO: 98 - protein, 29 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(29): Clostridium tetani TT1 Th
SIPWNLERITKKKKQYIKANSKFIGITEL

SEQ ID NO: 99 - protein, 34 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(34): EBV EBNA-1 Th
SIPWNLERITKKKKPGPLRESIVCYFMVFLQTHI

SEQ ID NO: 100 - protein, 35 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(35): Clostridium tetani TT2 Th
SIPWNLERITKKKKFNNFTVSFWLRVPKVSASHLE

SEQ ID NO: 101 - protein, 30 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(30): Clostridium tetani TT3 Th
SIPWNLERITKKKKKFIIKRYTPNNEIDSF

SEQ ID NO: 102 - protein, 30 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(30): Clostridium tetani TT4 Th
SIPWNLERITKKKKVSIDKFRIFCKALNPK

SEQ ID NO: 103 - protein, 32 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(32): EBV CP Th
SIPWNLERITKKKKVPGLYSPCRAFFNKEELL

SEQ ID NO: 104 - protein, 28 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(28): HCMV IE1 Th
SIPWNLERITKKKKDKREMWMACIKELH

SEQ ID NO: 105 - protein, 29 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(29): EBV GP340 Th
SIPWNLERITKKKKTGHGARTSTEPTTDY

SEQ ID NO: 106 - protein, 27 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(27): EBV BPLF1 Th
SIPWNLERITKKKKKELKRQYEKKLRQ

SEQ ID NO: 107 - protein, 25 aa, Artificial Sequence
  Peptide immunogen construct
  PEPTIDE (1)..(10): PCSK9 (153-162)
  PEPTIDE (11)..(14): epsilon-K-KKK spacer
  SITE (14)..(14): epsilon-K
  PEPTIDE (15)..(25): EBV EBNA-2 Th
SIPWNLERITKKKKTVFYNIPPMPL

SEQ ID NO: 108 - DNA, 32 bp, Artificial Sequence
  CpG1 oligonucleotide ODN
tcgtcgttttgtcgttttgtcgttttgtcgtt

SEQ ID NO: 109 - DNA, 24 bp, Artificial Sequence
  CpG2 oligonucleotide ODN
  misc_feature (1)..(1): phosphorothioate group
tcgtcgttttgtcgttttgtcgtt

SEQ ID NO: 110 - DNA, 24 bp, Artificial Sequence
  CpG3 Oligonucleotide ODN
tcgtcgttttgtcgttttgtcgtt

SEQ ID NO: 111 - protein, 300 aa, Homo sapiens
  PEPTIDE (1)..(300): Human PCSK9 catalytic domain
SIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAG

SEQ ID NO: 112 - protein, 300 aa, Macaca mulatta
  PEPTIDE (1)..(300): Monkey PCSK9 catalytic domain
SIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAG

SEQ ID NO: 113 - protein, 300 aa, Mus musculus
  PEPTIDE (1)..(300): Mouse PCSK9 catalytic domain
SIPWNLERIIPAWHQTEEDRSPDGSSQVEVYLLDTSIQGAHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNAACRHLARTGVVLVAAAGNFRDDACLYSPASAPEVITVGATNAQDQPVTLGTLGTNFGRCVDLFAPGKDIIGASSDCSTCFMSQSGTSQAAAHVAGIVARMLSREPTLTLAELRQRLIHFSTKDVINMAWFPEDQQVLTPNLVATLPPSTHETG

SEQ ID NO: 114 - protein, 300 aa, Rattus norvegicus
  PEPTIDE (1)..(300): Rat PCSK9 catalytic domain
SIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETG

SEQ ID NO: 115 - protein, 300 aa, Cavia porcellus
  PEPTIDE (1)..(300): Guinea pig PCSK9 catalytic domain
SVPWNLERILPVRHQAKEYSAPRGSHPVTVYLLDTSIQSGHREIQGRITVTDFESVPQEDGTRFHRQASKCDSHGTHLAGVVSGRDAGVAKGAGLRSLRVLNCQGRGTVSSTLRGLEFIRKSQLAQPVEPLVVLLPLAGGYSRTLNAACHLLARAGVVLVAAAGNFRDDACLYSPASAPEVITVGATNAQDQPVTLGTLGTNFGRCVDLFAPGEDIIGASSDCSTCFVSRSGTSQAAAHVAGIVTMMLTAQPKLTLAELWQRLIHFAAKDVINEAWFPEDQRVLTPNLVATLPPSTRGAG